(19) United States
(12) Patent Application Publication
(10) Pub. No.: US 2011/0098269 A1
(43) Pub. Date: Apr. 28, 2011

(54) Substituted Pyridazinone Derivatives as Histamine-3 (H3) Receptor Ligands

(75) Inventors: Nadine C. Becknell, Coatesville, PA (US); Reddeppa reddy Dandu, Downingtown, PA (US); Derek D. Dunn, Coatesville, PA (US); Robert L. Hudkins, Chester Springs, PA (US); Kurt A. Josef, Radnor, PA (US); Babu G. Sundar, West Chester, PA (US); Allison L. Zulli, Wayne, PA (US)

(73) Assignee: Cephalon, Inc., Frazer, PA (US)

(21) Appl. No.: 12/948,496

(22) Filed: Nov. 17, 2010

Related U.S. Application Data
(63) Continuation of application No. PCT/US2009/003122, filed on May 20, 2009.
(60) Provisional application No. 61/128,248, filed on May 20, 2008.

(51) Int. Cl. A61K 31/501; C07D 403/10; C07D 401/14; C07D 403/14; C07D 237/14; C07D 401/10; C07D 413/10; A61K 31/55; A61K 31/4985; A61K 31/5377; A61P 3/00; A61P 3/04; A61P 25/00; A61P 25/20; A61P 25/28; A61P 25/18; A61P 25/22; A61P 25/06; A61P 25/04; A61P 25/24; A61P 25/08; A61P 29/00; A61P 9/10; A61P 1/00; A61P 11/00

(52) U.S. Cl. 514/210.2; 540/594; 544/238; 544/239; 544/114; 514/217.01; 514/252.05; 514/252.02; 514/249; 514/252.03; 514/236.5

(57) ABSTRACT

The present invention provides compounds according to Formulas I, II, III, IV, V, VI, VII or VIII:

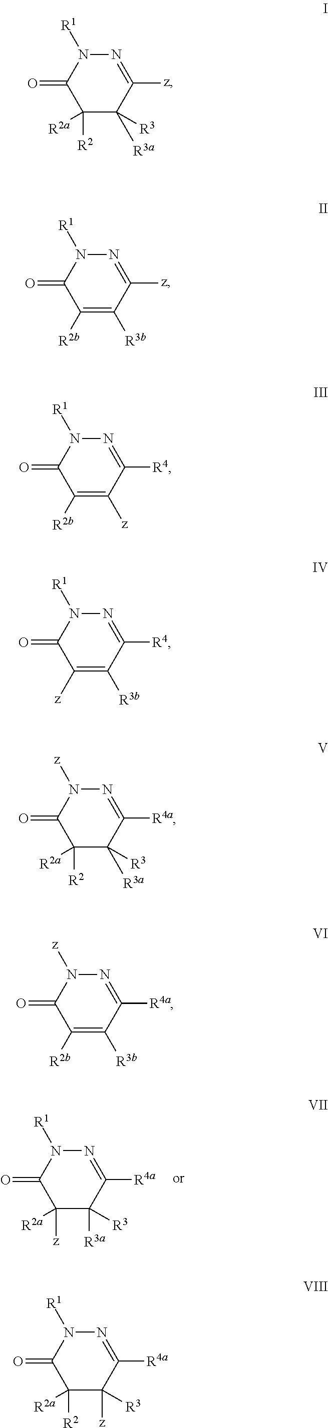

their use as H₃ antagonists/inverse agonists, processes for their preparation, and pharmaceutical compositions thereof.

### CROSS-REFERENCE TO RELATED APPLICATIONS

This application is a continuing application of International Application Serial No. PCT/US2009/003122, filed May 20, 2009, which claims the benefit of U.S. Provisional Application Ser. No. 61/128,248, filed May 20, 2008, the disclosure of which is incorporated herein by reference in its entirety.

### FIELD OF THE INVENTION

The present invention is related to substituted pyridazinone derivatives, their use as H₃ antagonists/inverse agonists, processes for their preparation, and pharmaceutical compositions thereof.

### BACKGROUND OF THE INVENTION

Publications cited throughout this disclosure are incorporated in their entirety herein by reference.

Histamine is a well established modulator of neuronal activity. At least four subtypes of histamine receptors have been reported in the literature—H₁, H₂, H₃, H₄. The histamine H₃ receptors play a key role in neurotransmission in the central nervous system. The H₃ receptor was discovered in 1983 originally on histamine-containing neurons where it was shown to function presynaptically, regulating the release and synthesis of the biogenic amine histamine (Arrang et al, 1983) now a well established neurotransmitter. H₃ receptors are predominately expressed in the brain, localizing to the cerebral cortex, amygdala, hippocampus, striatum, thalamus and hypothalamus. H₃ receptors are also localized presynaptically on histaminergic nerve terminals and act as inhibitory autoreceptors (Alguacil and Perez-Garcia, 2003; Passani et al, 2004; Leurs at al, 2005; Celanire et al, 2005; Witkin and Nelson, 2004). When these receptors are activated by histamine, histamine release is inhibited. H₃ receptors can also be found in the periphery (skin, lung, cardiovascular system, intestine, GI tract, etc). H₃ receptors are also involved in presynaptic regulation of the release of acetylcholine, dopamine, GABA, glutamate and serotonin (see Repka-Ramirez, 2003; Chazot and Hann, 2001; Leurs et al, 1998). The H₃ receptor demonstrates a high degree of constitutive or spontaneous activity (e.g., receptor is active in the absence of agonist stimulation) in vitro and in vivo, thus, ligands to the receptor can display, agonist, neutral antagonist or inverse agonist effects.

The location and function of histaminergic neurons in the CNS suggests that compounds interacting with the H₃ receptor may have utility in a number of therapeutic applications including narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders and epilepsy (Leurs et al, 2005; Witkin and Nelson, 2004, Hancock and Fox 2004; Esbenshade et al. 2006). An H₃ antagonist/inverse agonist could be important for gastrointestinal disorders, respiratory disorders such as asthma, inflammation, and myocardial infarction.

Ohtake et al. (US 2006/0178375 A1) disclosed compounds that reportedly exhibit histamine receptor H₃ antagonist or inverse agonist activity and may be useful for the treatment or prevention of obesity, diabetes, hormonal secretion abnormality, or sleep disorders.

Celanire et al. (WO 2006/103057 A1 and WO 2006/103045) have disclosed compounds comprising an oxazoline or thiazoline moiety, processes for preparing them, their pharmaceutical compositions and their uses as H₃ ligands.

Bertrand et al. (WO 2006/117609 A2) disclosed novel histamine H₃ receptor ligands, processes for their preparation, and their therapeutic applications.

Schwartz et al. (WO 2006/103546 A2) disclosed certain methods of treatment for Parkinson's disease, obstructive sleep apnea, narcolepsy, dementia with Lewy bodies, and/or vascular dementia using non-imidazole alkylamine derivatives that are antagonists of the H₃ receptors of histamine.

Apodaca et al. (EP 1 311 482 B1) disclosed certain non-imidazole aryloxypiperidines as H₃ receptor ligands, their synthesis, and their use for the treatment of disorders and conditions mediated by the histamine receptor.

Xu et al. disclosed certain 6-substituted phenyl-4,5-dihydro-3(2H)-pyridazinones, their synthesis, and rabbit platelet aggregation inhibitory activity induced by ADP in vitro.

Barker et al. (US 2006/0217375) discloses spiro[benzodioxane] compounds as active antagonists of the orexin-1 receptor and potentially useful in the prophylaxis and treatment of orexin-1 receptor related disorders and orexin-2 receptor related disorders.

Thus, there is a need for novel classes of compounds that possess the beneficial properties. It has been discovered that currently disclosed class of compounds, referred to herein as substituted pyridazinone derivatives, are useful as agents for treating or preventing various diseases or disorders disclosed herein.

### SUMMARY OF THE INVENTION

The present invention in one aspect is directed to novel compounds which are useful as H₃ antagonists/inverse agonists. These compounds have the structures illustrated below:

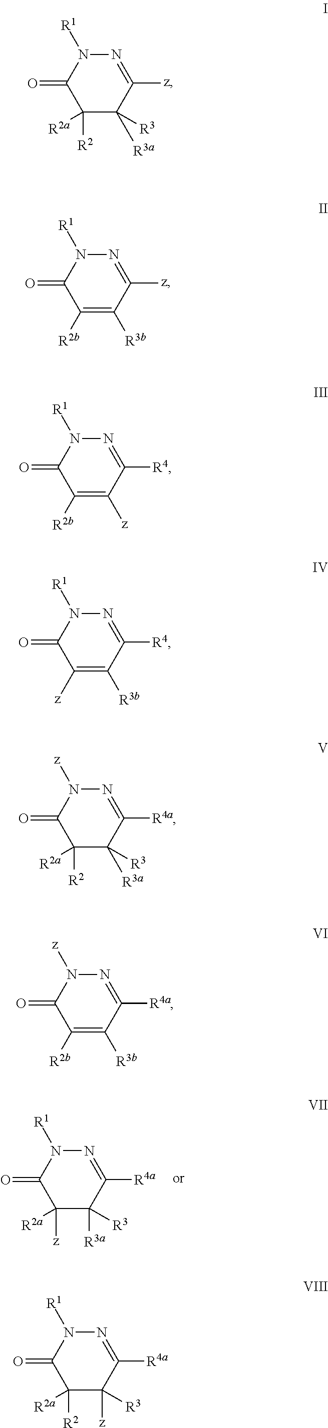

and include all stereoisomers, pharmaceutically acceptable salt forms, solvates and prodrugs thereof. More specifically the novel compounds are substituted pyridazin-3-ones.

The compounds of the present invention may be used to treat the following diseases and disorders: narcolepsy or other sleep/wake disorders, such as obstructive sleep apnea/hypopnea syndrome, and shift work sleep disorder; feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders (such as asthma), inflammation, and myocardial infarction.

In another aspect, the present invention is directed to a pharmaceutical composition which comprises a pharmaceutically acceptable carrier and a compound of the present invention, preferably in a therapeutically effective amount.

### DETAILED DESCRIPTION OF THE INVENTION

Thus, in a first embodiment, the present invention provides novel compounds or stereoisomers or pharmaceutically acceptable salts or solvates or prodrugs thereof according to Formulas I, II, III, IV, V, VI, VII or VIII:

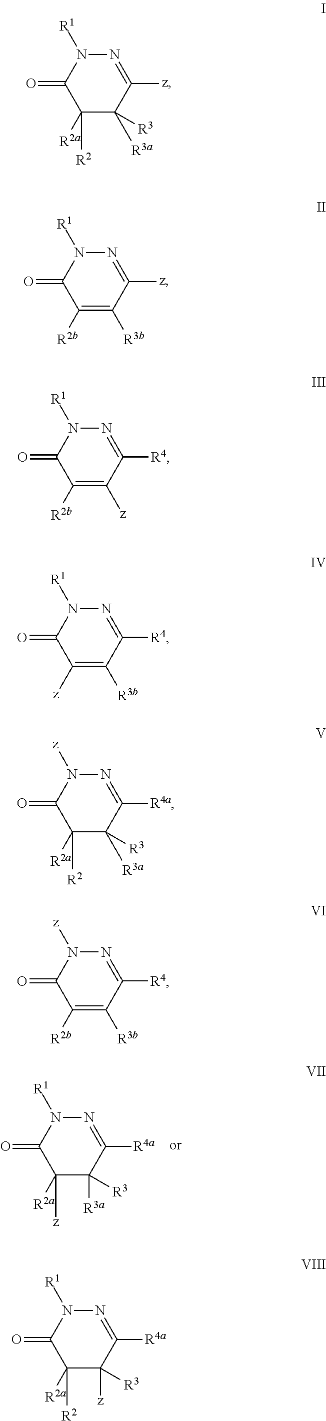

wherein

R¹ is selected from the group consisting of H, C₁₋₆ alkyl, C₂₋₆ alkenyl, C₂₋₆ alkynyl, C₃₋₁₀ cycloalkyl, C₆₋₁₀ aryl, C₇₋₁₈ arylalkyl, 5-10 membered heteroaryl, 3-10 membered heterocycloalkyl, —C(O)R²⁷ and —CO₂R²⁷, wherein the alkyl, cycloalkyl, aryl, arylalkyl, heteroaryl or heterocycloalkyl group is optionally substituted with 1 to 3 R²⁰ groups;

R², R³, R⁴, R^{2a}, R^{2b}, R^{3a}, R^{3b} and R^{4a} are independently selected from the group consisting of H, halo, C₁₋₆ alkyl, C₆₋₁₀ aryl, C₇₋₁₈ arylalkyl, C₁₋₆ alkoxy, —S(O)_{y}—C₁₋₆ alkyl, OR¹¹, C(O)R¹¹, CO₂R¹¹, C(O)NR¹²R¹³ and NR¹²R¹³, C₃₋₁₀ cycloalkyl, 3-10 membered heterocycloalkyl and 5-10 membered heteroaryl, alternatively R^{2b} and R^{3b} or R^{3b} and R⁴ may be taken together to form a C₃₋₁₀ cycloalkyl, 3-10 membered heterocycloalkyl, C₆₋₁₀ aryl or a 5-10 membered heteroaryl; or R² and R³ or R^{2a} and R^{3a} or R^{2a} and R³ or R² and R^{3a} or R^{3a} and R^{4a} or R² and R^{2a} or R³ and R^{3a} or R³ and R^{4a} may be taken together to form a C₃₋₁₀ cycloalkyl or 3-10 membered heterocycloalkyl; provided that no more than one pair of R² and R³, R³ and R^{4a}, R^{2a} and R^{3a}, R^{2a} and R³, R² and R^{3a}, R^{3a} and R^{4a}, R² and R^{2a}, R³ and R^{3a}, R^{2b} and R^{3b} or R^{3b} and R⁴ are taken together with the carbon atoms through which they are connected or to which they are attached to form a ring; and wherein the fused cycloalkyl, heterocycloalkyl, aryl or heteroaryl ring is optionally substituted with 1 to 3 R²⁰ groups.

Z is selected from the group consisting of

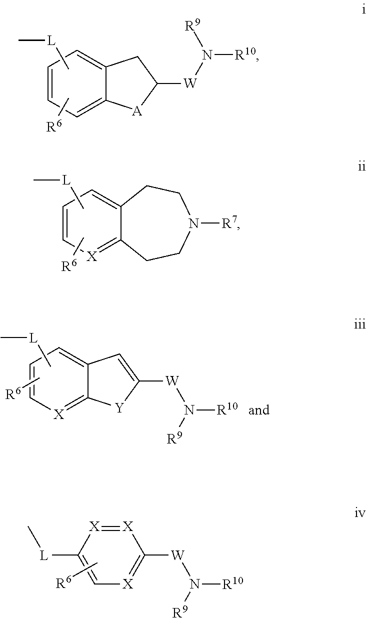

A is selected from the group consisting of C₁₋₃ alkylene, —CH₂—O— and —O—CH₂—;

L is selected from the group consisting of a direct bond, —R²⁵—O— and —O—R²⁵—;

W is selected from the group consisting of a bond, C₁₋₆ alkylene, C₃₋₁₀ cycloalkylene, C₆₋₁₀ arylene and —C(O)—;

X is independently selected from the group consisting of —C(H)— and —N—;

Y is selected from the group consisting of —N(R³¹)—, —S—, —O—, —C(H)═C(H)— and —C(H)—N(R³¹)—, provided that Y is not —S—, —O— or —C(H)═C(H)— for compounds according to Formulas V and VI;

R⁶ is selected from the group consisting of H, halo, CN, NO₂, C₁₋₆ alkyl, C₂₋₆ alkenyl, C₂₋₆ alkynyl, C₃₋₆ cycloalkyl, 3-10 membered heterocycloalkyl, C₆₋₁₀ aryl, 5-10 membered heteroaryl, C₁₋₆ alkoxy, C₁₋₆ haloalkyl, OR¹¹, C(O)R¹¹, CO₂R¹¹, C(O)NR¹²R¹³ and NR¹²R¹³;

R⁷ is selected from the group consisting of H, C₁₋₆ alkyl, C₂₋₆ alkenyl, C₂₋₆ alkynyl and C₃₋₁₀ cycloalkyl;

R⁹ and R¹⁰ are each independently selected from the group consisting of H, C₁₋₆ alkyl and C₃₋₆ cycloalkyl, wherein said alkyl and cycloalkyl groups may be optionally substituted with 1 to 3 R¹⁴ groups, alternatively R⁹ and R¹⁰ may together with the nitrogen to which they are attached form a 3-10 membered mono- or bicyclo-heterocycloalkyl ring, said heterocycloalkyl ring may be optionally substituted with 1 to 3 R¹⁴ groups;

R¹¹ is selected from the group consisting of H, C₁₋₆ haloalkyl, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said haloalkyl, alkyl, alkenyl, alkynyl, cycloalkyl, aryl, heteroaryl and heterocycloalkyl groups may be optionally substituted with 1 to 3 R²¹ groups;

R¹² and R¹³ are each independently selected from the group consisting of H, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said alkyl, alkenyl, alkynyl, cycloalkyl, aryl, heteroaryl and heterocycloalkyl groups may be optionally substituted with 1 to 3 R²¹ groups, or R¹² and R¹³, together with the nitrogen atom to which they are attached, form a 3-10 membered heterocycloalkyl ring optionally substituted with 1 to 3 R²¹ groups;

R¹⁴ at each occurrence is independently selected from the group consisting of halo, NO₂, CN, —(═O), —C(O)R³⁰, —C(O)OR³⁰, —OC(O)R³⁰, —OC(O)NR²⁸R²⁹, —C(O)NR²⁹R²⁹, —SR³⁰, —S(O)R³⁰, —S(O)₂R³⁰, —S(O)₂NR²⁸R²⁹, NR²⁸R²⁹, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said alkyl, alkenyl, alkynyl, cycloalkyl, aryl, heteroaryl and heterocycloalkyl groups may be optionally substituted with 1 to 3 R²¹ groups;

R²⁰ at each occurrence is independently selected from the group consisting of F, Cl, Br, I, OR²¹, OR²², NR²³R²⁴, NHOH, NO₂, CN, CF₃, C₁₋₆ alkyl optionally substituted with OR²⁶, C₂₋₆ alkenyl, C₂₋₆ alkynyl, C₃₋₁₀ cycloalkyl, 3-10 membered heterocycloalkyl, C₄₋₁₈ cycloalkylalkyl, 4-18 membered heterocycloalkylalkyl, phenyl, 6-18 membered heteroarylalkyl, C₇₋₁₈ arylalkyl, (═O), C(═O)R²¹, CO₂R²¹, OC(═O)R²¹, C(═O)NR²³R²⁴, NR²⁷C(═O)R²¹, NR²⁷C(═O)OR²¹, OC(═O)NR²³R²⁴, NR²⁷C(═S)R²¹ and S(O)_{q}R²¹;

R²¹ at each occurrence is independently selected from the group consisting of H, C₁₋₆ alkyl, C₆₋₁₀ aryl, 3-10 membered heterocycloalkyl and C₇₋₁₈ arylalkyl;

R²² is independently the residue of an amino acid after the hydroxyl group of the carboxyl group is removed;

R²³ and R²⁴ are independently selected from the group consisting of H, C₁₋₆ alkyl and C₆₋₁₀ aryl, or R²³ and R²⁴, together with the nitrogen atom to which they are attached, form a 3-10 membered heterocycloalkyl ring optionally substituted with ═O;

R²⁵ is selected from the group consisting of a direct bond, C₁₋₆ alkylene, C₆₋₁₀ arylene and 5-10 membered heteroarylene;

R²⁶ is selected from the group consisting of H, C₁-C₆ alkyl, C₆₋₁₀ aryl and C₇₋₁₈ arylalkyl;

R²⁷ is selected from the group consisting of H and C₁-C₆ alkyl;

R²⁸ and R²⁹ are each independently selected from the group consisting of H, C₁₋₆ alkyl and C₃₋₆ cycloalkyl or R²⁸ and R²⁹ may together with the nitrogen to which they are attached form a 3-10 membered mono- or bicyclo-heterocycloalkyl ring;

R³⁰ is selected from the group consisting of H, C₁₋₆ haloalkyl, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl;

R³¹ is selected from the group consisting of H, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said alkyl, alkenyl, alkynyl, cycloalkyl, aryl, heteroaryl and heterocycloalkyl groups may be optionally substituted with 1 to 3 R²¹ groups,

q is 0, 1 or 2; and

y is 0, 1 or 2.

Specific embodiments of the present invention exist when the following combinations of Formulas I-VIII and variables Z occur, and variables R¹, R², R³, R⁴, R^{2a}, R^{2b}, R^{3a}, R^{3b}, R^{4a}, Z, A, L, W, X, Y, R⁶, R⁷, R⁹ and R¹⁰ are as described herein:

| Formula I-VIII | Variable Z (i-iv) |
|---|---|
| I | i |
| I | ii |
| I | iii |
| I | iv |
| II | i |
| II | ii |
| II | iii |
| II | iv |
| III | i |
| III | ii |
| III | iii |
| III | iv |
| IV | i |
| IV | ii |
| IV | iii |
| IV | iv |
| V | i |
| V | ii |
| V | iii |
| V | iv |
| VI | i |
| VI | ii |
| VI | iii |
| VI | iv |
| VII | i |
| VII | ii |
| VII | iii |
| VII | iv |
| VIII | i |
| VIII | ii |
| VIII | iii |
| VIII | iv |

In one embodiment the present invention provides compounds of Formula I, wherein variables R¹, R², R^{2a}, R³, R^{3a}, and Z are as described herein.

In one embodiment the present invention provides compounds of Formula II, wherein variables R¹, R^{2b}, R^{3b}, and Z are as described herein.

In one embodiment the present invention provides compounds of Formula III, wherein variables R¹, R^{2b}, R⁴, and Z are as described herein.

In one embodiment the present invention provides compounds of Formula IV, wherein variables R¹, R^{3b}, R⁴, and Z are as described herein.

In one embodiment the present invention provides compounds of Formula V, wherein variables R², R^{2a}, R³, R^{3a}, R^{4a}, and Z are as described herein.

In one embodiment the present invention provides compounds of Formula VI, wherein variables R^{2b}, R^{3b}, R⁴, and Z are as described herein.

In one embodiment the present invention provides compounds of Formula VII, wherein variables R¹, R^{2a}, R³, R^{3a}, R^{4a}, and Z are as described herein.

In one embodiment the present invention provides compound of Formula VIII, wherein variables R¹, R², R^{2a}, R^{3a}, R^{4a}, and Z are as described herein.

In an additional embodiment, the present invention provides novel compounds according to Formulas I, II, III, IV, V, VI, VII or VIII, wherein

R^{2b}, R^{3b} and R⁴ are independently selected from the group consisting of H, halo, C₁₋₆ alkyl, C₆₋₁₀ aryl, C₇₋₁₈ arylalkyl, C₁₋₆ alkoxy, —S(O)_{y}—C₁₋₆ alkyl, OR¹¹, C(O)R¹¹, CO₂R¹¹, C(O)NR¹²R¹³ and NR¹²R¹³, C₃₋₁₀ cycloalkyl, 3-10 membered heterocycloalkyl and 5-10 membered heteroaryl; and

R², R^{2a}, R³, R^{3a} and R^{4a} are independently selected from the group consisting of H and C₁₋₆ alkyl, alternatively R^{2b} and R^{3b} or R^{3b} and R⁴ may be taken together to form a C₃₋₁₀ cycloalkyl, 3-10 membered heterocycloalkyl, C₆₋₁₀ aryl or a 5-10 membered heteroaryl; or R^{2a} and R^{3a} or R^{2a} and R³ or R² and R^{3a} or R^{3a} and R^{4a} or R² and R^{2a} or R³ and R^{3a} or R² and R³ or R³ and R^{4a} may be taken together to form a C₃₋₁₀ cycloalkyl or 3-10 membered heterocycloalkyl; provided that no more than one pair of R^{2b} and R^{3b}, R^{3b} and R⁴, R^{2a} and R^{3a}, R^{2a} and R³, R² and R^{3a}, R^{3a} and R^{4a}, R² and R^{2a} or R³ and R^{3a} or R² and R³ or R³ and R^{4a}, are taken together with the carbon atoms through which they are connected or to which they are attached to form a ring; and wherein the fused cycloalkyl, heterocycloalkyl, aryl or heteroaryl ring is optionally substituted with 1 to 3 R²⁰ groups.

In another embodiment, the present invention provides novel compounds or stereoisomers or pharmaceutically acceptable salts or solvates or prodrugs thereof according to Formulas I, II, III or IV, wherein

R¹ is selected from the group consisting of H, C₁₋₆ alkyl, C₃₋₁₀ cycloalkyl, C₆₋₁₀ aryl and 5-10 membered heteroaryl, wherein the alkyl, cycloalkyl, aryl, heteroaryl group is optionally substituted with 1 to 3 R²⁰ groups;

R^{2b}, R^{3b} and R⁴ are independently selected from the group consisting of H, halo, C₁₋₆ alkyl, C₆₋₁₀ aryl, 5-10 membered heteroaryl and C₃₋₁₀ cycloalkyl;

R², R^{2a}, R³ and R^{3a} are independently selected from the group consisting of H and C₁₋₆ alkyl, or R² and R³ or R² and R^{2a} or R^{2b} and R^{3b} or R^{3b} and R⁴ may be taken together to form a C₃₋₁₀ cycloalkyl; provided that no more than one pair of R² and R³ or R² and R^{2a} or R^{2b} and R^{3b} or R^{3b} and R⁴ are taken together with the carbon atoms through which they are connected or to which they are attached to form a ring; and

R⁶ is selected from the group consisting of H, halo, CN, NO₂, C₁₋₆ alkyl, 5-10 membered heteroaryl, C₁₋₆ alkoxy, C₁₋₆ haloalkyl, OR¹¹, C(O)R¹¹, CO₂R¹¹, C(O)NR¹²R¹³ and NR¹²R¹³.

Preferred compounds of the present invention are those of Formulas I, II and III.

Further preferred compounds of the present invention exist, wherein

Y is selected from the group consisting of —N(R³¹)—, —S— and —C(H)═C(H)—.

Additional preferred compounds of the present invention exist when,

W is —C(O)—.

Still further, preferred compounds of the present invention exist, when

R¹ is selected from the group consisting of H and C₁₋₆ alkyl.

Still further, preferred compounds of the present invention exist, when

R^{2b}, R^{3b} and R⁴ are independently selected from the group consisting of H, C₁₋₆ alkyl, C₆₋₁₀ aryl, 5-10 membered heteroaryl and C₃₋₁₀ cycloalkyl; and

R², R^{2a}, R³ and R^{3a} are independently selected from the group consisting of H and C₁₋₆ alkyl, or R^{2b} and R^{3b} or R^{3b} and R⁴ may be taken together to form a C₃₋₁₀ cycloalkyl; provided that no more than one pair of R^{2b} and R^{3b} or R^{3b} and R⁴ are taken together with the carbon atoms through which they are connected or to which they are attached to form a ring.

Still more preferred compounds of the present invention exist, when

R¹, R², R³, R^{2a}, R^{2b}, R^{3a}, R^{3b}, R⁴ and R^{4a} are independently selected from the group consisting of H and C₁₋₆ alkyl, or R^{2b} and R^{3b} or R^{3b} and R⁴ may be taken together to form a C₃₋₁₀ cycloalkyl or a C₆₋₁₀ aryl; provided that no more than one pair of R^{2b} and R^{3b} or R^{3b and R}⁴ are taken together with the carbon atoms through which they are connected or to which they are attached to form a ring.

Preferably, R¹, R², R³, R^{2a}, R^{2b}, R^{3a}, R^{3b}, R⁴ and R^{4a} are independently selected from the group consisting of H and C₁₋₆ alkyl, or R² and R³ or R^{2a} and R³ or R³ and R^{4a} may be taken together to form a C₃₋₁₀ cycloalkyl; provided that no more than one pair of R² and R³ or R^{2a} and R³ or R³ and R^{4a} are taken together with the carbon atoms through which they are connected or to which they are attached to form a ring.

Preferably, X is —C(H)—.

Additional preferred compounds exist when either R³ or R⁴ is a 5-10 membered heteroaryl.

Other preferred compounds of the present invention exist, when R⁹ and R¹⁰ are taken together with the nitrogen to which they are attached to form a pyrrolidinyl ring or piperidinyl ring, wherein said ring may be optionally substituted with 1 to 3 R¹⁴ groups.

Preferably, R¹⁴ at each occurrence is independently selected from the group consisting of C₁₋₆ alkyl and 3-10 membered heterocycloalkyl, wherein said alkyl and heterocycloalkyl groups may be optionally substituted with 1 to 3 R²¹ groups. More preferably, R¹⁴ at each occurrence is independently selected from the group consisting of C₁₋₆ alkyl and 3-10 membered heterocycloalkyl, wherein said alkyl group may be optionally substituted with 1 to 3 R²¹ groups. More preferably, R¹⁴ at each occurrence is independently selected from the group consisting of C₁₋₆ alkyl and 3-10 membered heterocycloalkyl, wherein said alkyl and heterocycloalkyl groups may be optionally substituted with 1 to 3 R²¹ groups, wherein R²¹ is 3-10 membered heterocycloalkyl. More preferably, R¹⁴ at each occurrence is independently selected from the group consisting of C₁₋₆ alkyl and 3-10 membered heterocycloalkyl, wherein said alkyl group may be optionally substituted with 1 to 3 R²¹ groups, wherein R²¹ is 3-10 membered heterocycloalkyl. More preferably, R¹⁴ at each occurrence is independently selected from the group consisting of C₁₋₆ alkyl, pyrrolidinyl and piperidinyl, wherein said alkyl, pyrrolidinyl and piperidinyl groups may be optionally substituted with 1 to 3 R²¹ groups, wherein R²¹ is 3-10 membered heterocycloalkyl. More preferably, R¹⁴ at each occurrence is independently selected from the group consisting of C₁₋₆ alkyl, pyrrolidinyl and piperidinyl, wherein said alkyl group may be optionally substituted with 1 to 3 R²¹ groups, wherein R²¹ is 3-10 membered heterocycloalkyl. More preferably, R¹⁴ at each occurrence is independently selected from the group consisting of C₁₋₆ alkyl, pyrrolidinyl and piperidinyl, wherein said alkyl, pyrrolidinyl and piperidinyl groups may be optionally substituted with 1 to 3 R²¹ groups, wherein R²¹ is selected from the group consisting of pyrrolidinyl and piperidinyl. More preferably, R¹⁴ at each occurrence is independently selected from the group consisting of C₁₋₆ alkyl, pyrrolidinyl and piperidinyl, wherein said alkyl group may be optionally substituted with 1 to 3 R²¹ groups, wherein R²¹ is selected from the group consisting of pyrrolidinyl and piperidinyl. More preferably, R¹⁴ at each occurrence is independently selected from the group consisting of methyl, pyrrolidinyl and piperidinyl, wherein said methyl, pyrrolidinyl and piperidinyl groups may be optionally substituted with 1 to 3 R²¹ groups, wherein R²¹ is selected from the group consisting of pyrrolidinyl and piperidinyl. More preferably, R¹⁴ at each occurrence is independently selected from the group consisting of methyl, pyrrolidinyl and piperidinyl, wherein said methyl group may be optionally substituted with 1 to 3 R²¹ groups, wherein R²¹ is selected from the group consisting of pyrrolidinyl and piperidinyl. More preferably, R¹⁴ is pyrrolidinyl or piperidinyl.

Preferably, Z is a group of formula iii or iv.

Embodiments of the present invention exist when,

R¹ is selected from the group consisting of H, C₁₋₆alkyl, C₃₋₁₀ cycloalkyl, C₆₋₁₀ aryl, C₇₋₁₈ arylalkyl, 5-10 membered heteroaryl, 3-10 membered heterocycloalkyl, C(═O)R²⁷ and CO₂R²⁷, wherein the alkyl, cycloalkyl, aryl, arylalkyl, heteroaryl, or heterocycloalkyl group is optionally substituted with 1 to 3 R²⁰ groups;

R^{2a}, R^{3b} and R⁴ are each independently selected from the group consisting of H, C₁₋₆ alkyl, C₆₋₁₀ aryl, C₇₋₁₈ arylalkyl C₁₋₆ alkoxyl, S(═O)_{y}—C₁₋₆ alkyl, C₃₋₁₀ cycloalkyl, 3-10 membered heterocycloalkyl and 5-10 membered heteroaryl;

R², R^{2a}, R³, R^{3a} and R^{4a} are each indepedently selected from the group consisting of H and C₁₋₆ alkyl; or R^{2b} and R^{3b} or R^{3b} and R⁴, taken together with the carbon atoms through which they are connected form a fused phenyl, thienyl, pyrrolyl, oxazolyl, pyridinyl, or C₃₋₆ cycloalkyl ring; or R² and R³ or R² and R^{3a} or R³ and R^{4a} or R^{2a} and R^{3a} or R^{2a} and R³ or R^{3a} and R^{4a} or R² and R^{2a} or R³ and R^{3a}, taken together with the carbon atoms through which they are connected form a fused C₃₋₁₀ cycloalkyl ring; or R² and R^{2a} or R³ and R^{3a} taken together with the carbon atoms through which they are connected form a fused C₃₋₈ cycloalykl; provided that no more than one pair of R² and R³, R^{2b} and R^{3b}, R^{3b} and R⁴, R³ and R^{4a}, R^{2a} and R^{3a}, R^{2a} and R³, R² and R^{3a}, R^{3a} and R^{4a}, R² and R^{2a} or R³ and R^{3a}, are taken together with the carbon atoms through which they are connected or to which they are attached to form a ring; and wherein the fused phenyl, thienyl, pyrrolyl, oxazolyl, pyridinyl, or cycloalkyl ring is optionally substituted with 1 to 3 R²⁰ groups.

Embodiments of the present invention exist according to Formula I, II, III, IV or VI,

wherein

R¹ is selected from the group consisting of H, C₁₋₆alkyl, C₃₋₁₀ cycloalkyl, C₆₋₁₀ aryl, C₇₋₁₈ arylalkyl, 5-10 membered heteroaryl, 3-10 membered heterocycloalkyl, C(═O)R²⁷ and CO₂R²⁷, wherein the alkyl, cycloalkyl, aryl, arylalkyl, heteroaryl, or heterocycloalkyl group is optionally substituted with 1 to 3 R²⁰ groups;

R^{2a}, R^{2b}, R^{3a}, R^{3b} and R⁴ are each independently selected from the group consisting of H, C₁₋₆ alkyl, C₆₋₁₀ aryl and C₇₋₁₈ arylalkyl, or R^{2b} and R^{3b} or R^{3b} and R⁴ or R^{2a} and R^{3a} taken together with the carbon atoms through which they are connected form a fused phenyl, thienyl, pyrrolyl, cyclopentyl or cyclohexyl ring; wherein the phenyl, thienyl, pyrrolyl, cyclopentyl or cyclohexyl rings are optionally substituted with 1 to 3 R²⁰ groups; and

Z, R²⁰ and R²⁷ are as defined herein.

Preferred embodiments of the present invention are illustrated below:

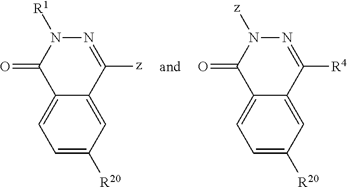

wherein

R¹ is selected from the group consisting of H, C₁₋₆ alkyl, phenyl and benzyl, wherein the alkyl, phenyl and benzyl group are optionally substituted with 1 to 3 R²⁰ groups; and

R¹, R²⁰ and Z are as defined herein.

Embodiment of the present invention exist according to Formulas I, II or VI:

wherein

R¹ is selected from the group consisting of H, C₁₋₆ alkyl, phenyl and benzyl, wherein the alkyl, phenyl and benzyl groups are optionally substituted with 1 to 3 R²⁰ groups;

R^{2a} and R^{3a} are each independently selected from the group consisting of H; and

R^{2b}, R^{3b} and R⁴ are each independently selected from the group consisting of H C₁₋₆ alkyl, phenyl and benzyl, or R^{2b} and R^{3b} or R^{3b} and R⁴ can combine to form a fused phenyl, thienyl, pyrrolyl, cyclopentyl or cyclohexyl ring; wherein the phenyl, thienyl, pyrrolyl, cyclopentyl or cyclohexyl rings are optionally substituted with 1 to 3 R²⁰ groups.

Embodiments of the present invention include those compounds where Formula I is:

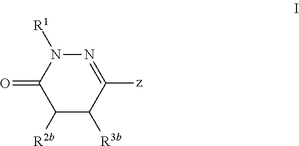

wherein R¹, R^{2b}, R^{3b} and Z are as defined herein.

Embodiments of the present invention include those compounds where Formula II is:

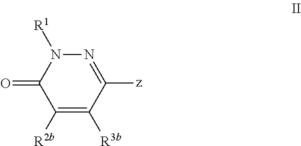

wherein R¹, R^{2b}, R^{3b} and Z are as defined herein.

Embodiments of the present invention include those compounds where Formula III is:

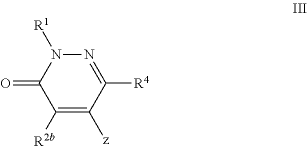

wherein R¹, R^{2b} and Z are as defined herein.

Embodiments of the present invention include those compounds where Formula IV is:

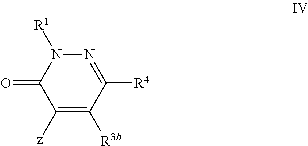

wherein R¹, R^{3b} and Z are as defined herein.

Embodiments of the present invention include those compounds where Formula VI is:

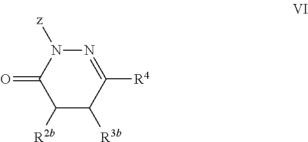

wherein R¹, R^{2b}, R^{3b} and Z are as defined herein.

In some preferred embodiments Formula I is:

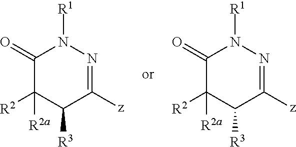

wherein,

R¹, R², R^{2a}, R³ and Z are as defined herein.

In some preferred embodiments, R¹ is selected from the group consisting of H, C₁₋₆ alkyl, C₃₋₁₀ cycloalkyl, C₆₋₁₀ aryl, C₇₋₁₈ arylalkyl and 5-10 membered heteroaryl, more preferably H or 5-10 membered heteroaryl; with 5-10 membered heteroaryl being even more preferred. Alternatively, C₁₋₆ alkyl, C₆₋₁₀ aryl, C₇₋₁₈ arylalkyl or 5-10 membered heteroaryl is preferred in some embodiments.

In some preferred embodiments, R², R^{2a}, R^{2b}, R³, R^{3a}, R^{3b}, R⁴ and R^{4a} are each independently selected from the group consisting of H and C₁₋₆ alkyl, more preferably wherein at least one of is R², R^{2a}, R^{2b}, R³, R^{3a}, R^{3b}, R⁴ and R^{4a} is C₁₋₆ alkyl. In other alternatively preferred embodiments, R², R^{2a} and R^{2b} are each independently C₁₋₃ alkyl; or R³, R^{3a} and R^{3b} are each independently C₁₋₃ alkyl.

In some preferred embodiments, R^{2b} and R^{3b}, taken together with the carbon atoms through which they are connected form a fused phenyl, thienyl, pyrrolyl, oxazolyl, pyridinyl, or C₃₋₆ cycloalkyl ring; or R^{3b} and R⁴ or R³ and R^{4a} or R^{2a} and R^{3a} or R^{2a} and R³ or R² and R^{3a} or R^{3a} and R^{4a} taken together with the carbon atoms through which they are connected form a fused C₃₋₆ cycloalkyl ring; or R² and R^{2a} or R³ and R^{3a} taken together with the carbon atoms through which they are connected form a fused C₃₋₈ cycloalkyl ring; provided that no more than one pair of R^{2b} and R^{3b}, R^{3b} and R⁴, R³ and R^{4a}, R^{2a} and R^{3a}, R^{2a} and R³, R² and R^{3a}, R^{3a} and R^{4a}, R² and R^{2a} or R³ and R^{3a}, are taken together with the carbon atoms through which they are connected or to which they are attached to form a ring; and wherein the fused phenyl, thienyl, pyrrolyl, oxazolyl, pyridinyl, or cycloalkyl ring is optionally substituted with 1 to 3 R²⁰ groups. More preferably, R^{2b} and R^{3b} or R^{2a} and R³ or R² and R^{3a} or R^{2a} and R^{3a} or R³ and R^{4a}, taken together with the carbon atoms through which they are connected form a fused C₃₋₆ cycloalkyl ring; or R² and R^{2a}, or R³ and R^{3a}, taken together with the carbon atom to which they are attached form a C₃₋₈ cycloalkyl ring; then the cycloalkyl ring is a C₃₋₄ cycloalkyl ring.

In some preferred embodiments of the present invention, R³ or R⁴ is heteroaryl.

In some preferred embodiments of the present invention, R²⁰ is C₁₋₆ alkyl; in others it is C₃₋₁₀ cycloalkyl, more preferably cyclobutyl. In still others, R²⁰ is F, Cl, CF₃, NR²³R²⁴, or C₁₋₆ alkyl optionally substituted with OR²⁶, C₄₋₁₈ cycloalkylalkyl, or 4-18 membered heterocycloalkylalkyl, more preferably C₁₋₆ alkyl optionally substituted with OR²⁶.

In some preferred embodiments R²³ and R²⁴ are each independently C₁₋₆ alkyl.

In some preferred embodiments R²⁶ is selected from the group consisting of H and C₁₋₆ alkyl.

Preferred embodiments of the present invention exist according to Formulas I, II or III when,

R¹ is selected from the group consisting of H, C₁₋₆ alkyl, phenyl and benzyl,

wherein the alkyl, phenyl and benzyl groups are optionally substituted with 1 to 3 R²⁰ groups; and

R², R^{2b}, R³ and R^{3b} are each independently selected from the group consisting of H, C₁₋₆ alkyl, phenyl, and benzyl, or R² and R³ or R^{2b} and R^{3b} can combine to form a fused phenyl, thienyl, pyrrolyl, cyclopentyl or cyclohexyl ring; wherein the phenyl, thienyl, pyrrolyl, cyclopentyl or cyclohexyl rings are optionally substituted with 1 to 3 R²⁰ groups.

More preferred embodiments of the present invention exist according to Formulas I or II when,

R^{2a} and R^{3a} are each H; and

R², R^{2a}, R^{2b}, R³, R^{3a} and R^{3b} are each independently selected from the group consisting of H, C₁₋₆ alkyl, phenyl, and benzyl, or R² and R³ or R^{2b} and R^{3b} can combine to form a fused phenyl, thienyl, cyclopropyl, cyclobutyl, cyclopentyl or cyclohexyl ring.

Preferred embodiments of the present invention exist according to Formulas I or II when,

R^{2a} and R^{3a} are each H; and

R², R^{2b}, R³ and R^{3b} and each independently selected from the group consisting of H, C₁₋₆ alkyl, phenyl and benzyl.

Preferred embodiments of the present invention exist according to Formulas I or II when,

R^{2a} and R^{3a} are each H; and

R² and R³ or R^{2b} and R^{3b} combine to form a fused cyclopropyl, cyclobutyl, cyclopentyl or cyclohexyl ring.

Preferred embodiments of the present invention exist according to Formulas I or II when,

R^{2a} and R^{3a} are each H; and

R² and R³ or R^{2b} and R^{3b} combine to form a fused phenyl or thienyl ring.

In another embodiment, the present invention includes compounds of the following formula:

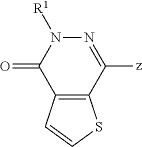

wherein each variable is defined in herein. Additional embodiments include compounds with the preferred variables as described herein.

In another embodiment, the present invention includes compounds of the following formula:

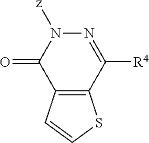

wherein each variable is defined in herein. Additional embodiments include compounds with the preferred variables as described herein.

In another embodiment, the present invention includes compounds of the following formula:

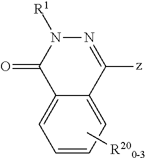

wherein each variable is defined in herein. Additional embodiments include compounds with the preferred variables as described herein.

In another embodiment, the present invention includes compounds of the following formula:

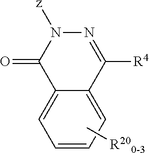

wherein each variable is defined in herein. Additional embodiments include compounds with the preferred variables as described herein.

In another embodiment, the present invention includes compounds of the following formula:

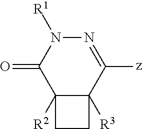

wherein each variable is defined in herein. Additional embodiments include compounds with the preferred variables as described herein.

In another embodiment, the present invention includes compounds of the following formula:

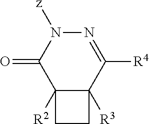

wherein each variable is defined in herein. Additional embodiments include compounds with the preferred variables as described herein.

In another embodiment, the present invention includes compounds of the following formula:

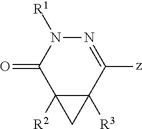

wherein each variable is defined in herein. Additional embodiments include compounds with the preferred variables as described herein.

In another embodiment, the present invention includes compounds of the following formula:

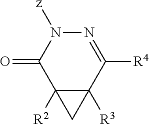

wherein each variable is defined in herein. Additional embodiments include compounds with the preferred variables as described herein.

In a second embodiment the present invention provides a pharmaceutical composition comprising a compound according to the present invention and one or more pharmaceutically acceptable excipients. The pharmaceutical composition may optionally contain one or more additional therapeutic agents.

In a third embodiment the present invention provides for a method for treating a disorder selected from the group consisting of narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders, inflammation, and myocardial infarction comprising administering to a subject in need of such treatment a therapeutically effective amount of at least one compound of the present invention. In a preferred embodiment the present invention provides for a method of treating narcolepsy or sleep/wake disorders. In a preferred embodiment the present invention provides for a method of treating attention deficit hyperactivity disorder. In a preferred embodiment the present invention provides for a method of treating cognition. Optionally, the method of the present invention may contain one or more additional therapeutic agents that may be administer before, after or concurrently with the at least one compound of the present invention.

In a fourth embodiment the present invention provides for use of the compounds of the present invention for use in therapy.

In a fifth embodiment the present invention provides for use of the compounds of the present invention in the manufacture of a medicament for treating a disorder selected from the group consisting of narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders, inflammation, and myocardial infarction comprising administering to a subject in need of such treatment a therapeutically effective amount of a compound of the present invention. Optionally, the medicament may contain one or more additional therapeutic agents.

### DEFINITIONS

In the formulas described and claimed herein, it is intended that when any symbol appears more than once in a particular formula or substituent, its meaning in each instance is independent of the other.

The following terms and expressions have the indicated meanings.

As used herein, the term “about” refers to a range of values from ±10% of a specified value. For example, the phrase “about 50” includes ±10% of 50, or from 45 to 55. The phrase “from about 10 to 100” includes ±10% of 10 and ±10% of 100, or from 9 to 110.

As used herein, a range of values in the form “x-y” or “x to y”, or “x through y”, include integers x, y, and the integers therebetween. For example, the phrases “1-6”, or “1 to 6” or “1 through 6” are intended to include the integers 1, 2, 3, 4, 5, and 6. Preferred embodiments include each individual integer in the range, as well as any subcombination of integers. For example, preferred integers for “1-6” can include 1, 2, 3, 4, 5, 6, 1-2, 1-3, 1-4, 1-5, 2-3, 2-4, 2-5, 2-6, etc.

As used herein “stable compound” or “stable structure” refers to a compound that is sufficiently robust to survive isolation to a useful degree of purity from a reaction mixture, and preferably capable of formulation into an efficacious therapeutic agent. The present invention is directed only to stable compounds.

As used herein, “substituted” refers to any one or more hydrogen atoms on the indicated atom is replaced with a selected group referred to herein as a “substituent”, provided that the substituted atom's valency is not exceeded, and that the substitution results in a stable compound.

As used herein, the term “alkyl” refers to a straight-chain, or branched alkyl group having 1 to 8 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, octyl, etc. The alkyl moiety of alkyl-containing groups has the same meaning as alkyl defined above. A designation such as “C₁-C₆ alkyl” refers to straight-chain, or branched alkyl group having 1 to 6 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, etc. Lower alkyl groups, which are preferred, are alkyl groups as defined above which contain 1 to 4 carbons. A designation such as “C₁-C₄ alkyl” refers to an alkyl radical containing from 1 to 4 carbon atoms, such as methyl, ethyl, propyl, isopropyl, butyl, isobutyl, sec-butyl, and tert-butyl. A designation such as “C₁-C₃ alkyl” refers to an alkyl radical containing from 1 to 3 carbon atoms, such as methyl, ethyl, propyl, and isopropyl. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “alkylene” refers to a bivalent “alkyl” group.

As used herein, the term “alkenyl” refers to a straight chain, or branched hydrocarbon chains of 2 to 6 carbon atoms having at least one carbon-carbon double bond. A designation “C₂-C₆ alkenyl” refers to an alkenyl radical containing from 2 to 6 carbon atoms. Examples of alkenyl groups include ethenyl, propenyl, isopropenyl, 2,4-pentadienyl, etc. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “alkenylene” refers to a bivalent “alkenyl” group. As used herein, the term “alkynyl” refers to a straight chain, or branched hydrocarbon chains of 2 to 6 carbon atoms having at least one carbon-carbon triple bond. A designation “C₂-C₆ alkynyl” refers to an alkynyl radical containing from 2 to 6 carbon atoms. Examples include ethynyl, propynyl, isopropynyl, 3,5-hexadiynyl, etc. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “alkynylene” refers to a bivalent “alkynyl” group.

As used herein, the term “haloalkyl” refers to an “alkyl” group as defined herein substituted by one or more halogen atoms to form a stable compound. Examples of haloalkyl, include but are not limited to, —CF₃, —CHF₂ and —CH₂F.

As used herein, the term “alkoxy” refers to an “alkyl” group as defined herein bonded to and oxygen atom. Examples of alkoxy, include but are not limited to, methoxy and ethoxy.

As used herein, the term “halo” refers to an F, Cl, Br, and I. Preferred halo substituents are F and Cl.

As used herein, the term “cycloalkyl” refers to a saturated or partially saturated mono- or bicyclic alkyl ring system containing 3 to 10 carbon atoms. Certain embodiments contain 3 to 6 carbon atoms, and other embodiments contain 5 or 6 carbon atoms. A designation such as “C₃-C₁₀ cycloalkyl” refers to a cycloalkyl radical containing from 3 to 10 ring carbon atoms. Examples of cycloalkyl groups include such groups as cyclopropyl, cyclobutyl, cyclopentyl, cyclohexyl, and cycloheptyl. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “cycloalkylene” refers to a bivalent “cycloalkyl” group.

As used herein, the term “cycloalkylalkyl” refers to a “cycloalkyl” group as defined herein bonded through an “alkylene” group as defined herein, containing 4 to 18 carbon atoms in total. Examples of cycloalkylalkyl, include but are not limited to, cyclohexylmethyl and 2-cyclopentylethyl.

As used herein, the term “aryl” refers to a substituted or unsubstituted, mono- or bicyclic hydrocarbon aromatic ring system having 6 to 10 ring carbon atoms. Examples include phenyl and naphthyl. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein, the term “arylene” refers to a bivalent “aryl” group.

As used herein, the term “arylalkyl” refers to an unsubstituted or substituted “aryl” group as defined herein bonded through an “alkylene” group as defined herein, containing 7 to 18 carbon atoms in total. Examples of arylalkyl, include but are not limited to, benzyl and phenethyl.

As used herein, the term “heteroaryl” refers to an aromatic group or ring system containing 5 to 10 ring carbon atoms in which one or more ring carbon atoms are replaced by at least one hetero atom such as O, N, or S. Certain embodiments include 5 or 6 membered rings. Examples of heteroaryl groups include pyrrolyl, furanyl, thienyl, pyrazolyl, imidazolyl, thiazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, picolinyl, imidazopyridinyl, indolyl, isoindolyl, indazolyl, benzofuranyl, isobenzofuranyl, purinyl, quinazolinyl, quinolyl, isoquinolyl, benzoimidazolyl, benzothiazolyl, benzothiophenyl, thianaphthenyl, benzoxazolyl, benzooxadiazolyl, benzisoxazolyl, cinnolinyl, phthalazinyl, naphthyridinyl, and quinoxalinyl. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “heteroarylene” refers to a bivalent “heteroaryl” group.

As used herein, the term “heteroaryalkyl” refers to an unsubstituted or substituted “heteroaryl” group as defined herein bonded through an “alkylene” group as defined herein, containing 6 to 18 atoms in total. Examples of heteroarylalkyl include but are not limited to, pyridinylmethyl and pyrrazolylmethyl.

As used herein, the term “heterocycloalkyl” refers to a cycloalkyl group in which one or more ring carbon atoms are replaced by at least one hetero atom such as O, N, S, SO, and SO₂. Certain embodiments include 3 to 6 membered rings, and other embodiments include 5 or 6 membered rings. Examples of heterocycloalkyl groups include azetidinyl, 3H-benzooxazolyl, 1,1-dioxo-thiomorpholinyl, 1,4-diazapinyl, pyrrolidinyl, pyrrolinyl, imidazolidinyl, imidazolinyl, imidazolidinyl, oxazolidinyl, pirazolidinyl, pirazolinyl, pyrazalinyl, piperidinyl, piperazinyl, hexahydropyrimidinyl, morpholinyl, thiomorpholinyl, dihydrobenzofuranyl, tetrahydrofuranyl, tetrahydropyranyl, tetrahydropyrazolopyridinyl, tetrahydro-1,3a,7-triaza-azulenyl, dihydro-oxazolyl, dithiolyl, oxathiolyl, dioxazolyl, oxathiazolyl, pyranyl, oxazinyl, oxathiazinyl, and oxadiazinyl. Included within the definition of “heterocycloalkyl” are fused ring systems, including, for example, ring systems in which an aromatic ring is fused to a heterocycloalkyl ring. Examples of such fused ring systems include, for example, phthalamide, phthalic anhydride, indoline, isoindoline, tetrahydroisoquinoline, chroman, isochroman, chromene, and isochromene. Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “heterocycloalkylene” refers to a bivalent “heterocycloalkyl” group.

As used herein, the term “heterocycloalkylalkyl” refers to an unsubstituted or substituted “heterocyloalkyl” group as defined herein bonded through an “alkylene” group as defined herein, containing 4 to 18 atoms in total. Examples of heterocycloalkylalkyl include but are not limited to, pyrrolidinylmethyl and piperidinylmethyl.

As used herein, the term “methylenedioxy”, “ethylenedioxy”, or “propylenedioxy” refer to a —O—CH₂—O—, —O—CH₂CH₂—O—, or —O—CH₂CH₂CH₂—O— group, respectively, bonded to a cycloalkyl, aryl, heteroaryl, or heterocycloalkyl moiety, as defined herein, through the two oxygen atoms of the methylenedioxy, ethylenedioxy, or propylenedioxy. The methylenedioxy, ethylenedioxy, or propylenedioxy groups may be bonded to the cyclic moiety through one carbon atom of the cyclic moiety (i.e. a spirocyclic bond) or through two adjacent carbons of the cyclic moiety (i.e. fused). Further, said groups, as defined herein, may optionally be substituted on any available atom with one or more functional groups commonly attached to such atom, such as, but not limited to hydroxyl, halo, haloalkyl, cyano, mercapto, alkylthio, heterocycloalkyl, aryl, heteroaryl, carboxyl, carbalkoyl, carboxamido, carbonyl, alkyl, alkenyl, alkynyl, nitro, amino, alkoxyl, aryloxyl, heteroaryloxyl, amido, and the like.

As used herein the term “residue of an amino acid after the hydroxyl group of the carboxyl group is removed” refers to a natural or artificial amino acid residue bonded through the carbon atom of the carbonyl group after removal of the acid's hydroxyl group. Examples of “residue of an amino acid after the hydroxyl group of the carboxyl group is removed”, include but are not limited to,

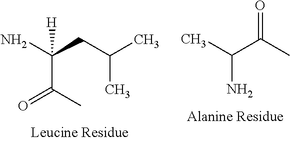

As used herein, the term “subject” refers to a warm blooded animal such as a mammal, preferably a human, or a human child, which is afflicted with, or has the potential to be afflicted with one or more diseases and conditions described herein.

As used herein, a “therapeutically effective amount” refers to an amount of a compound of the present invention effective to prevent or treat the symptoms of particular disorder. Such disorders include, but are not limited to, those pathological and neurological disorders associated with the aberrant activity of the receptors described herein, wherein the treatment or prevention comprises inhibiting, inducing, or enhancing the activity thereof by contacting the receptor with a compound of the present invention.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, the term “unit dose” refers to a single dose which is capable of being administered to a patient, and which can be readily handled and packaged, remaining as a physically and chemically stable unit dose comprising either the active compound itself, or as a pharmaceutically acceptable composition, as described hereinafter.

All other terms used in the description of the present invention have their meanings as is well known in the art.

In another aspect, the present invention is directed to pharmaceutically acceptable salts of the compounds described above. As used herein, “pharmaceutically acceptable salts” includes salts of compounds of the present invention derived from the combination of such compounds with non-toxic acid or base addition salts.

Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

Base addition salts include those derived from inorganic bases such as ammonium and alkali and alkaline earth metal hydroxides, carbonates, bicarbonates, and the like, as well as salts derived from basic organic amines such as aliphatic and aromatic amines, aliphatic diamines, hydroxy alkamines, and the like. Such bases useful in preparing the salts of this invention thus include ammonium hydroxide, potassium carbonate, sodium bicarbonate, calcium hydroxide, methylamine, diethylamine, ethylenediamine, cyclohexylamine, ethanolamine and the like.

In addition to pharmaceutically-acceptable salts, other salts are included in the invention. They may serve as intermediates in the purification of the compounds, in the preparation of other salts, or in the identification and characterization of the compounds or intermediates.

The pharmaceutically acceptable salts of compounds of the present invention can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present invention.

The present invention also encompasses the pharmaceutically acceptable prodrugs of the compounds disclosed herein. As used herein, “prodrug” is intended to include any compounds which are converted by metabolic processes within the body of a subject to an active agent that has a formula within the scope of the present invention. Since prodrugs are known to enhance numerous desirable qualities of pharmaceuticals (e.g., solubility, bioavailability, manufacturing, etc.) the compounds of the present invention may be delivered in prodrug form. Conventional procedures for the selection and preparation of suitable prodrug derivatives are described, for example, in Prodrugs, Sloane, K. B., Ed.; Marcel Dekker New York, 1992, incorporated by reference herein in its entirety

It is recognized that compounds of the present invention may exist in various stereoisomeric forms. As such, the compounds of the present invention include both diastereomers and enantiomers. The compounds are normally prepared as racemates and can conveniently be used as such, but individual enantiomers can be isolated or synthesized by conventional techniques if so desired. Such racemates and individual enantiomers and mixtures thereof form part of the present invention.

It is well known in the art how to prepare and isolate such optically active forms. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994, and Jacques, J, et al. Enantiomers, Racemates, and Resolutions; Wiley: New York, 1981, each incorporated by reference herein in their entireties.

It is further recognized that functional groups present on the compounds of Formula I may contain protecting groups. For example, the amino acid side chain substituents of the compounds of Formula I can be substituted with protecting groups such as benzyloxycarbonyl or t-butoxycarbonyl groups. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred groups for protecting lactams include silyl groups such as t-butyldimethylsilyl (“TBDMS”), dimethoxybenzhydryl (“DMB”), acyl, benzyl (“Bn”), and methoxybenzyl groups. Preferred groups for protecting hydroxy groups include TBS, acyl, benzyl, benzyloxycarbonyl (“CBZ”), t-butyloxycarbonyl (“Boc”), and methoxymethyl. Many other standard protecting groups employed by one skilled in the art can be found in Greene, T. W. and Wuts, P. G. M., “Protective Groups in Organic Synthesis” 2d. Ed., Wiley & Sons, 1991.

### Synthesis

The compounds of the present invention may be prepared in a number of methods well known to those skilled in the art, including, but not limited to those described below, or through modifications of these methods by applying standard techniques known to those skilled in the art of organic synthesis. All processes disclosed in association with the present invention are contemplated to be practiced on any scale, including milligram, gram, multigram, kilogram, multikilogram or commercial industrial scale.

As outline in the General Schemes 1-6, the pyridazinone examples may be synthesized by several methods including condensation of an 4-oxobutyric acid or ester intermediate of general structure I, or a derivative there of, with hydrazine or an R¹ N-substituted hydrazine derivative in a solvent such as ethanol or 2-propanol to provide a 4,5-dihydropyridazinone of general structure II. Keto-acid intermediates with substitution at the 4- and 5-position are known and may be readily prepared. Pyridazinones with R^{2/3}, R^{2a/3a}, R^{2a/3} or R^{2/3a} fused with heteroaryl or cycloalkyl groups are synthesized from the corresponding anhydrides or acid-esters. In cases where R¹ is a protecting group, deprotection gives R¹=H compounds. The 4,5-dihydropyridazinones II may be oxidized to an aromatic pyridazinone of general structure III using MnO₂, CuCl₂, DDQ, selenium oxide, DMSO/base or sodium 3-nitrobenzenesulfonate in the presence of sodium hydroxide. NH(R¹=H) pyridazinones may be alkylated with alkyl or substituted alkyl groups using an R¹-halide, a base, for example K₂CO₃, Cs₂CO₃ or NaH, in an inert solvent such as DMF, THF or CH₃CN. Examples wherein R¹ is H may be converted to analogs wherein R¹ is aryl or heteroaryl by standard palladium or copper coupling reactions using the appropriate aryl or heteroaryl halide.

X groups of the invention as described previously.

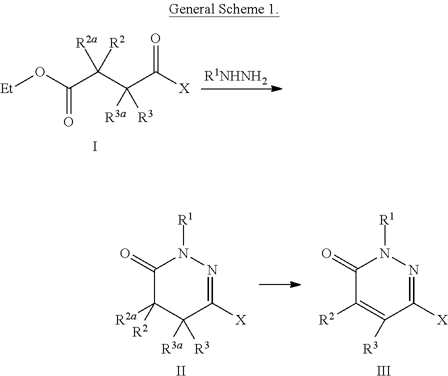

When X=naphthalene, examples may be prepared using methods described for example 51, examples 52-69, or by processes outlined in General Scheme 2 to synthesize additional pyridazinone examples of the invention, for example IV or V, using standard Suzuki cross-coupling chemistry. A boron ether derivative is subjected to a palladium catalyzed cross-coupling reaction (Suzuki reaction) with a pyridazine derivative of general structure VI or VII or a pyridazinone VIII where in the R^{2a}, R^{3a} or R⁴ group may be a halogen, preferably Br or Ito produce structures IV and V.

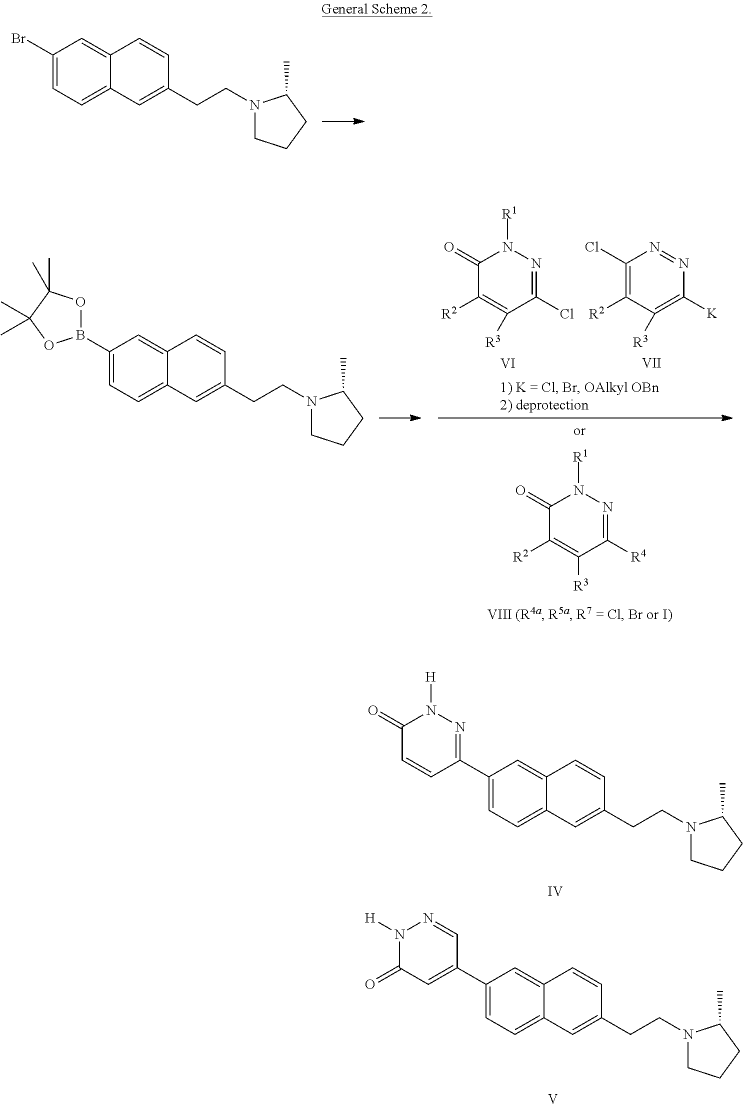

Pyridazinone-amides of the invention such as IX-XIV may be prepared using methods described for examples 46-50.

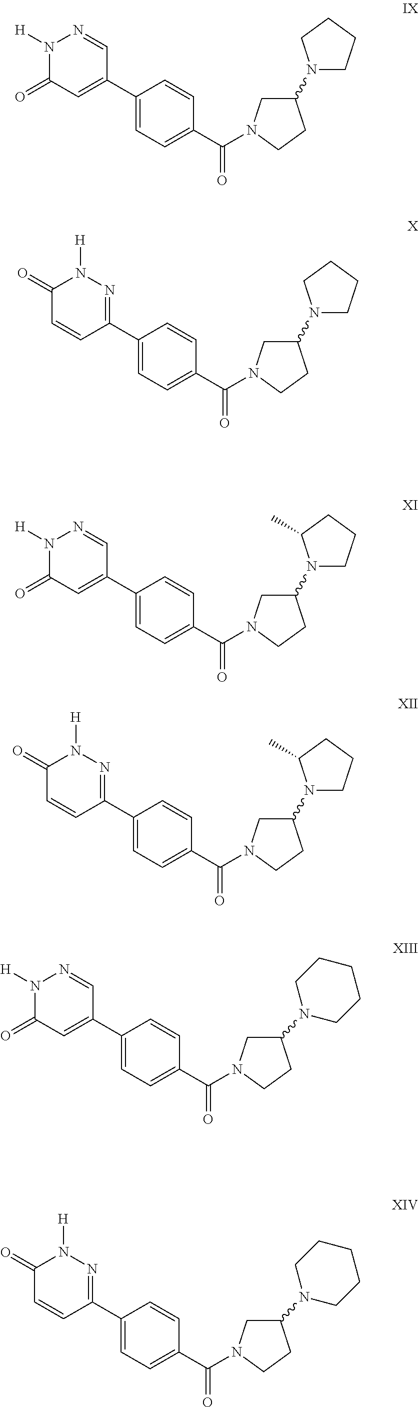

Pyridazinone-tetralines and pyridazinone-indanes examples may be synthesized by analogous manner to General Scheme 2 starting with a bromo-β-tetralone or a bromo-β-indazolneas outlined in General Scheme 3. Reductive amination conditions using the bromo-tetralone or bromo-indanone with a secondary amine such as piperidine, pyrrolidine or R-2-methylpyrrolidine and NaCNBH₃ or NaBH(OAc)₃ in an alcohol solvent provided a route to the bromo-amine intermediates. A palladium cross coupling reaction using, for example, tris(dibenzylideneacetone)dipalladium tricyclohexylphosphine, 4,4,5,5,4′,4′,5′,5′-octamethyl-[2,2′]bi[[1,3,2]dioxaborolanyl], potassium acetate and a bromo intermediate in dioxane may provide the boron ether such as XV. Suzuki cross-coupling reactions may be used to produce compounds such as XVI-XXI and the indanes.

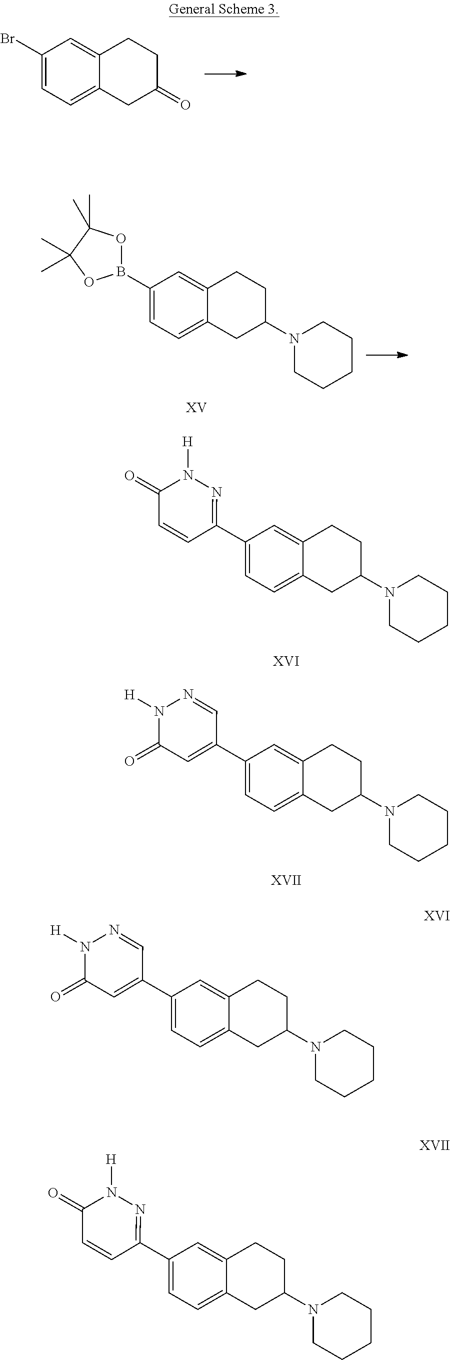

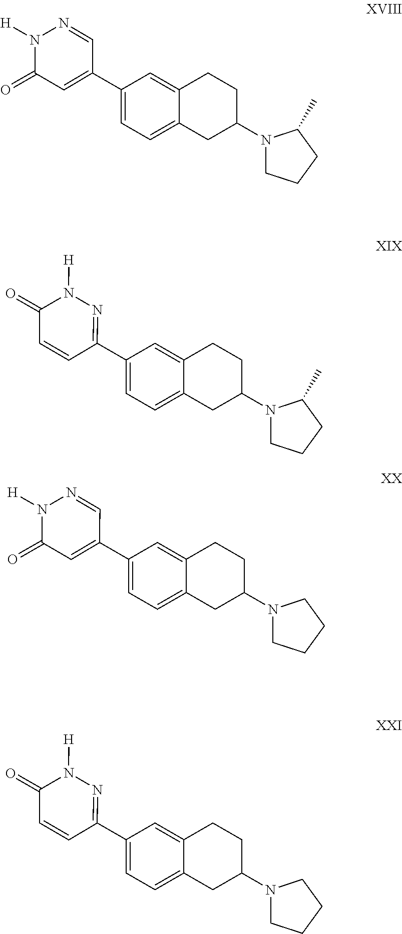

Examples of the invention where the pyridazinone general structure V-VI can be bonded through the N¹ nitrogen may be synthesized using standard copper coupling conditions such as Cu(0) in pyridine with K₂CO₃ or CuI in dioxane with 1,2-diaminocyclohexane and Cs₂CO₃ and the Z-bromide or Z-iodide.

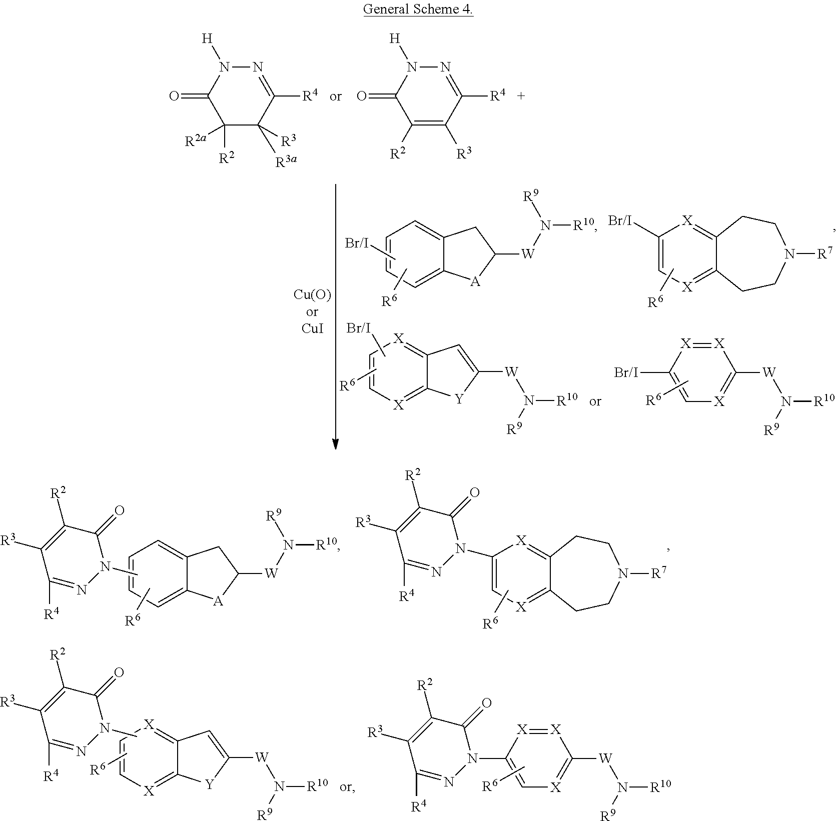

Pyridazinone examples of general structure VIII, where Z is attached to position 3a or 3b may be synthesized by reacting an appropriate Z-acetone (R⁴=Me) derivative (or R⁴ Z-β-ketone equivalent) with a base such as LiHMDS, KHMDS or NaH and ethyl α-bromo acetate in THF. The pyridazinone may be synthesized by treating the keto-ester product with R¹-hydrazine.

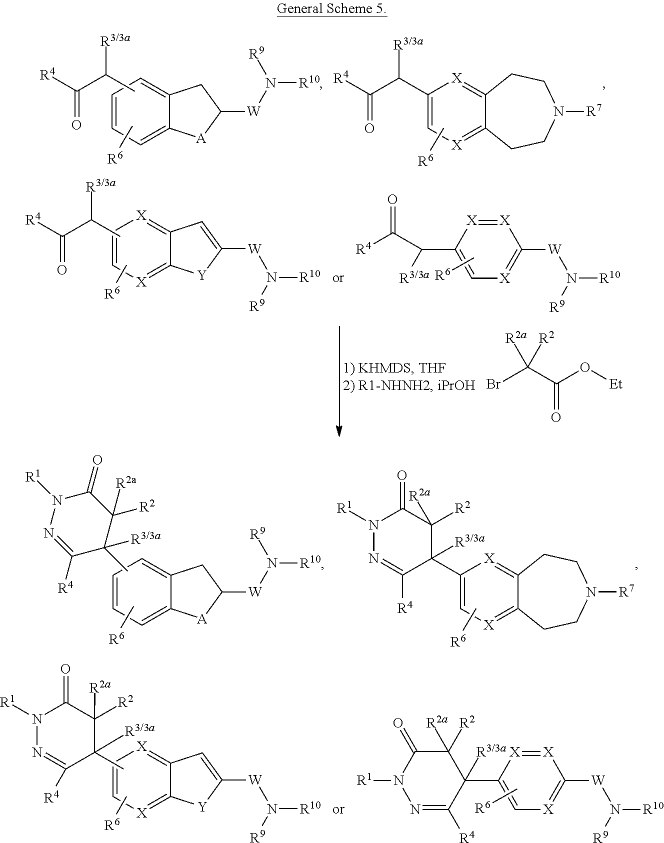

Pyridazinone examples of general structure VII, where Z is attached to position 2a or 2 may be synthesized in an analogous approach as VIII by reacting an appropriate Z-acetate derivative with a base such as LiHMDS, KHMDS or NaH and a α-bromo R⁴-ketone intermediate. The pyridazinone may be synthesized by treating the keto-ester product with R¹-hydrazine.

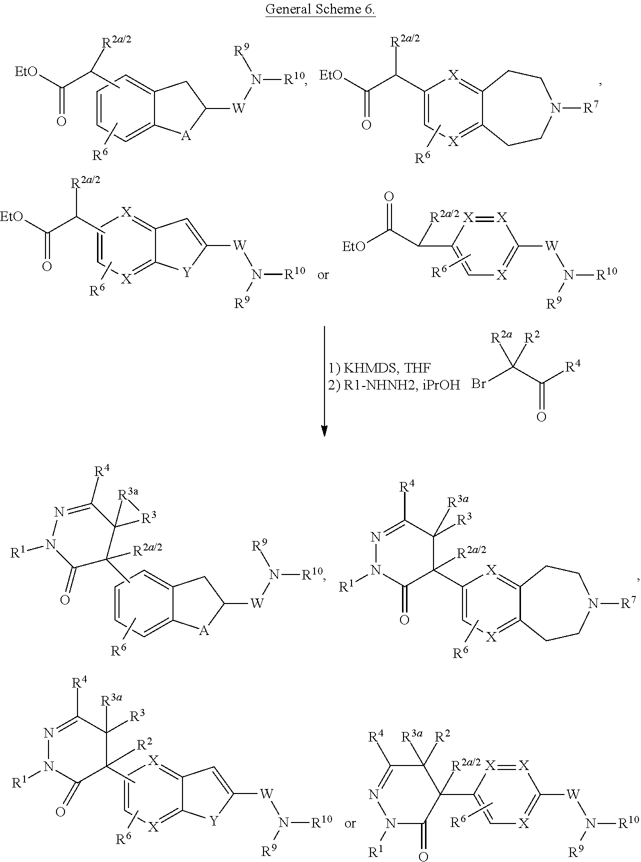

### EXAMPLES

Other features of the invention will become apparent in the course of the following descriptions of exemplary embodiments as shown below. The compounds shown herein have activity in the targets described herein at concentrations ranging from 0.1 nM to 10 μM. These examples are given for illustration of the invention and are not intended to be limiting thereof.

### Experimental Section

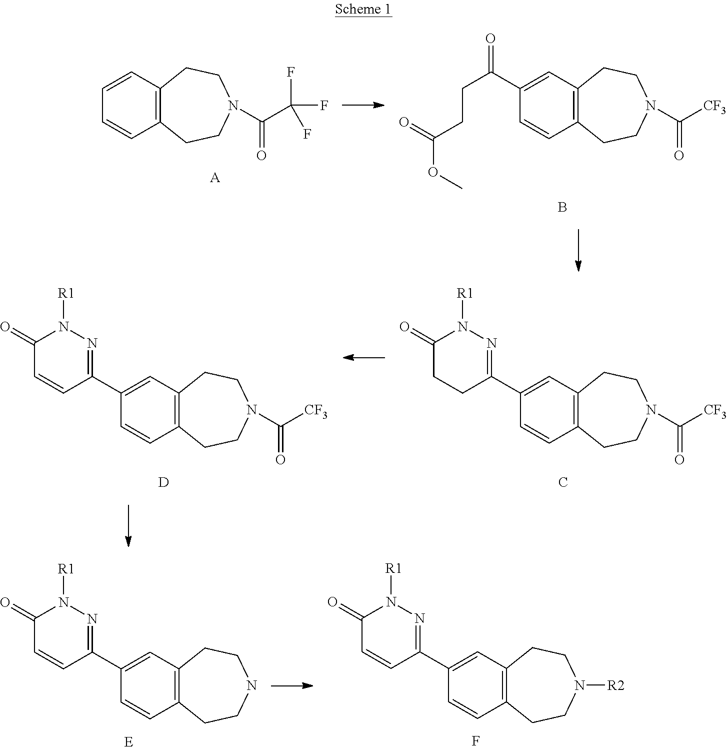

### Example 1

### 6-(2,3,4,5-Tetrahydro-1H-benzo[d]azepin-7-yl)-2H-pyridazin-3-one

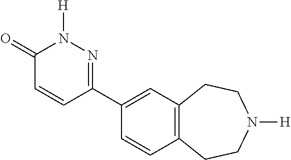

### Synthesis of Intermediate A, Scheme 1: 2,2,2-trifluoro-1-(1,2,4,5-tetrahydro-benzo[d]azepin-3-yl)-ethanone

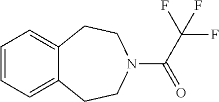

A solution of tetrahydro-1H-benzo[d]azepine (1.54 g, 10.4 mmol) in CH₂Cl₂ (15 mL) and pyridine (1.92 g, 24.3 mmol) was cooled at 0° C. and trifluoroacetic anhydride (2.55 g, 12.1 mmol) was added dropwise. The reaction mixture was further stirred at 0° C. for 3 h and quenched with 1N HCl. The aqueous layer was extracted twice with methylene chloride and the combined organics was washed with brine, dried (Na₂SO₄), filtered and concentrated under vacuum to afford a crude material. The crude material was purified by Biotage chromatography using 40 to 60% ethyl acetate in hexane to produce 2,2,2-trifluoro-1-(1,2,4,5-tetrahydro-benzo[d]azepin-3-yl)-ethanone, (1.62 g, 63%), mp 80-81° C. (ethyl acetate and hexane), MS m/z 244 (M+1).

### Step 1: Synthesis of Intermediate B, Scheme 1

To a solution of 2,2,2-trifluoro-1-(1,2,4,5-tetrahydro-benzo[d]azepin-3-yl)-ethanone (14.55 g, 61.6 mmol) in 1,2-dichloroethane (80 mL) at rt was added aluminum chloride (24.5 g, 184.8 mmol) and then slowly methyl 3-(chloroformyl)propionate (11.36 mL, 92.4 mmol). An LCMS sample after 3.5 h showed no starting material was present. The reaction was poured into 1N HCl/ice mixture and extracted with ethyl acetate. The ethyl acetate layer was washed with brine, dried (MgSO₄), filtered and concentrated to give 22 g (99%) of product, MS m/z=357 (M+1).

### Step 2: Synthesis of Intermediate C, Scheme 1

A solution of the product from step 1 (B) (1.9 g, 5.34 mmol) in isopropanol (20 mL) and hydrazine monohydrate (0.358 mL, 7.7 mmol) were heated in a sealed tube at 120° C. overnight. An LCMS sample showed no starting material was present. The reaction was cooled to rt and a precipitate formed. The solid was collected, washed with IPA and ether and dried. Yield=0.613 g (34%), MS m/z=340 (M+1).

### Step 3: Synthesis of Intermediate D, Scheme 1

To a solution of C (product from Example 1 step 2) (0.6 g, 1.77 mmol) in glacial acetic acid (30 mL) was added selenium dioxide (0.89 gm, 8 mmol). The reaction was heated to 125° C. for 2 h. An LCMS showed only product. The reaction was cooled to rt, diluted with CH₂Cl₂ and filtered through a celite pad. The celite was washed with 5% methanol/methylene chloride. The filtrate was concentrated and redissolved in 5% methanol/methylene chloride and saturated sodium bicarbonate added slowly. The organic layer was separated, dried (MgSO₄), filtered and concentrated. Yield=0.58 g (97%), MS m/z=338 (M+1).

### Step 4: Synthesis of Example 1

A solution of the product from Example 1 step 3 (0.58, 1.75 mmol), potassium carbonate (0.725 g, 5.25 mmol) and THF/MeOH (1:5) (12 mL) were stirred at rt for 6 h. An LCMS sample showed product was present. The reaction was concentrated, redissolved in 5% methanol/methylene chloride and washed with saturated sodium bicarbonate, brine, dried (MgSO₄), filtered and concentrated. Yield=210 mg (50%), MS m/z=242 (M+1).

### Example 2

### 6-(3-Cyclobutyl-2,3,4,5-tetrahydro-1H-benzo[d]azepin-7-yl)-2H-pyridazin-3-one

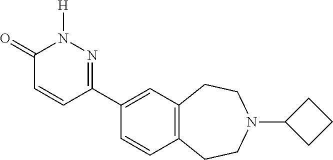

To a solution of the product from Example 1, step 4 (0.21 g, 0.87 mmol) in glacial acetic acid (3 drops) and methylene chloride (10 mL) was added cyclobutanone (0.1 mL, 1.31 mmol). After 45 minutes, sodium triacetoxyborohydride (0.28 g, 1.31 mmol) was added. An LCMS after 2 h showed no starting material. The reaction was quenched with 2 M sodium carbonate and the product extracted with methylene chloride. The organic layer was washed with brine, dried (MgSO₄), filtered and concentrated. The off-white solid was treated with ethyl ether, filtered and collected. The solid was stirred in 1:1 methylene chloride/ether filtered and collected. Yield=30 mg (15%). mp=250-251° C., MS=296 (M+1), HNMR (DMSO): 13.1 (1H, s), 8.0 (d, 1H), 7.6 (s, 1H), 7.5 (d, 1H), 7.2 (d, 1H), 7.0 (d, 1H), 2.9 (m, 4H), 2.75 (m, 1H), 2.35 (m, 4H), 2.0 (m, 2H), 1.8 (m, 2H), 1.6 (m, 2H).

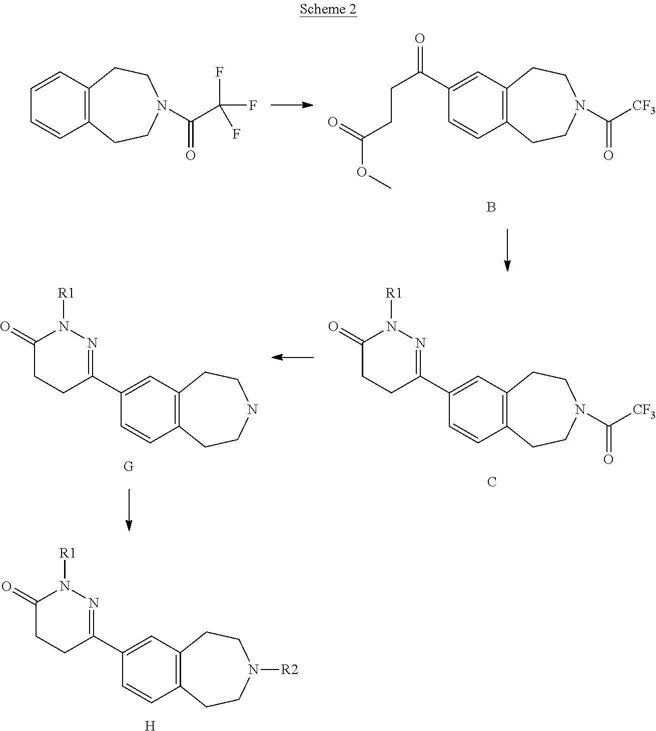

### Example 3

### 2-Isopropyl-6-(2,3,4,5-tetrahydro-1H-benzo[d]azepin-7-yl)-4,5-dihydro-2H-pyridazin-3-one

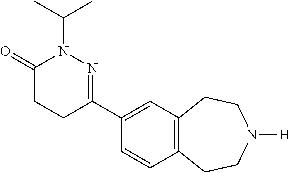

2-Isopropyl-6-[3-(2,2,2-trifluoro-acetyl)-2,3,4,5-tetrahydro-1H-benzo[d]azepin-7-yl]-4,5-dihydro-2H-pyridazin-3-one was prepared using isopropyl hydrazine by the method for C in step 2: MS m/z=382 (M+1). The product (0.7 g, 1.84 mmol), potassium carbonate (0.76 g, 5.81 mmol), and tetrahydrofuran:methanol (1:5) (12 mL) was stirred at rt overnight. The mixture was filtered and the filtrate concentrated. The residue was redissolved in 5% methanol/methylene chloride and washed with sat. sodium bicarbonate. The organic layer was separated, dried, filtered and concentrated. Yield=0.5 g (95%), oil at rt, MS m/z=286 (M+1), HNMR (CDCl₃): 7.55 (s, 1H), 7.50 (d, 1H), 7.15 (d, 1H), 5.0 (m, 1H), 3.0 (m, 8H), 2.9 (t, 2H), 2.5 (t, 2H), 2.1 (s, 1H), 1.25 (d, 6H).

### Example 4

### 6-(3-Cyclobutyl-2,3,4,5-tetrahydro-1H-benzo[d]azepin-7-yl)-2-isopropyl-4,5-dihydro-2H-pyridazin-3-one

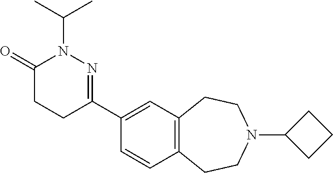

A solution of Example 3 (0.25 g, 0.877 mmol), cyclobutanone (0.1 mL, 1.32 mmol), sodium triacetoxyborohydride (0.28 g, 1.32 mmol), acetic acid (5 drops), and methylene chloride (5 mL) was stirred at rt for 2 h. The reaction was quenched with saturated sodium carbonate and extracted with methylene chloride. The organic layer was separated dried, filtered and concentrated. Yield=0.21 g (71%), mp=140-141° C., MS m/z=340 (M+1), HNMR (DMSO): 7.55 (s, 1H), 7.5 (d, 1H), 7.2 (d, 1H), 4.9 (m, 1H), 2.9 (m, 8H), 2.75 (m, 1H), 2.45 (t, 1H), 2.35 (m, 4H), 2.0 (m, 2H), 1.8 (m, 2H), 1.5 (m, 2H), 1.2 (d, 6H).

### Example 5

### 2-Isopropyl-6-(2,3,4,5-tetrahydro-1H-benzo[d]azepin-7-yl)-2H-pyridazin-3-one

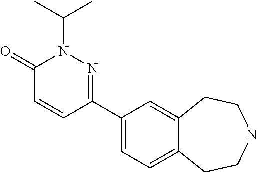

Synthesized via Scheme 1: oil at rt, MS m/z=284 (M+1), HNMR (CDCl₃): 7.65 (d, 1H), 7.6 (s, 1H), 7.55 (d, 1H), 7.2 (d, 1H), 7.0 (d, 1H), 5.4 (m, 1H), 3.0 (m, 8H), 1.4 (d, 6H).

### Example 6

### 2-Isopropyl-6-(3-isopropyl-2,3,4,5-tetrahydro-1H-benzo[d]azepin-7-yl)-4,5-dihydro-2H-pyridazin-3-one

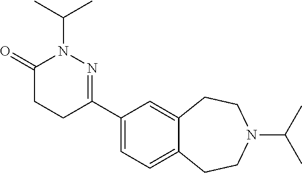

Synthesized via Scheme 2: mp=125-126° C., MS m/z=328 (M+1), HNMR (DMSO): 10.6 (bs, HCl), 7.7 (s, 1H), 7.6 (d, 1H), 7.3 (d, 1H), 4.9 (m, 1H), 3.5 (m, 5H), 3.0 (m, 6H), 2.45 (m, 2H), 1.3 (d, 6H), 1.2 (d, 6H).

### Example 7

### 6-(3-Cyclobutyl-2,3,4,5-tetrahydro-1H-benzo[d]azepin-7-yl)-2-isopropyl-2H-pyridazin-3-one

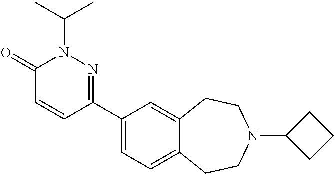

Synthesized via Scheme 1: mp=199-200° C., MS m/z=338 (M+1), HNMR (DMSO): 8.0 (d, 1H), 7.7 (s, 1H), 7.6 (d, 1H), 7.2 (d, 1H), 7.0 (d, 1H), 5.2 (m, 1H), 2.9 (m, 4H), 2.7 (m, 1H), 2.4 (m, 4H), 2.0 (m, 2H), 1.8 (m, 2H), 1.6 (m, 2H), 1.35 (d, 6H).

### Example 8

### 2-Isopropyl-6-(3-isopropyl-2,3,4,5-tetrahydro-1H-benzo[d]azepin-7-yl)-2H-pyridazin-3-one

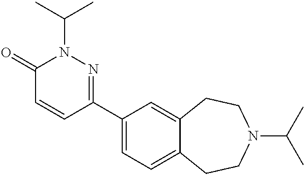

Synthesized via Scheme 1: mp=244-245° C., MS m/z=326 (M+1), HNMR (DMSO): 10.8 (bs, HCl), 8.0 (d, 1H), 7.8 (s, 1H), 7.7 (d, 1H), 7.4 (d, 1H), 7.0 (d, 1H), 5.2 (m, 1H), 3.6 (m, 4H), 3.0 (m, 5H), 1.4 (d, 6H), 1.3 (d, 6H).

### Example 9

### 6-(3-Cyclobutyl-2,3,4,5-tetrahydro-1H-benzo[d]azepin-7-yl)-4,5-dihydro-2H-pyridazin-3-one

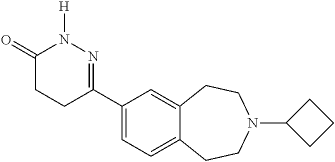

Synthesized via Scheme 2: mp=178-179° C., MS=298 (M+1), HNMR (DMSO): 10.8 (s, 1H), 7.5 (s, 1H), 7.45 (d, 1H), 7.2 (d, 1H), 2.9 (m, 6H), 2.8 (m, 1H), 2.4 (t, 2H), 2.3 (m, 4H), 2.0 (m, 2H), 1.8 (m, 2H), 1.6 (m, 2H).

### Example 10

### 6-(3-Cyclobutyl-2,3,4,5-tetrahydro-1H-benzo[d]azepin-7-yl)-2-(2,2,2-trifluoro-ethyl)-4,5-dihydro-2H-pyridazin-3-one

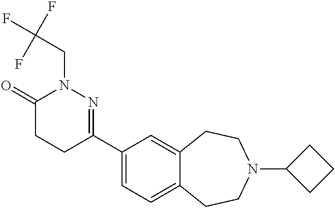

Synthesized via Scheme 2: mp=139-140° C., MS=380 (M+1), HNMR (DMSO): 7.55 (s, 1H), 7.5 (d, 1H), 7.2 (d, 1H), 4.6 (q, 2H), 3.0 (t, 2H), 2.9 (m, 4H), 2.7 (m, 1H), 2.5 (t, 2H), 2.4 (m, 4H), 2.0 (m, 2H), 1.8 (m, 2H), 1.6 (m, 2H).

### Example 11

### 6-(3-Cyclobutyl-2,3,4,5-tetrahydro-1H-benzo[d]azepin-7-yl)-2-(2,2,2-trifluoro-ethyl)-2H-pyridazin-3-one

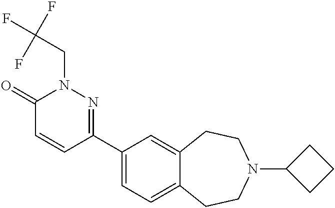

Synthesized via Scheme 1: mp=142-143° C., MS m/z=378 (M+1), HNMR (DMSO): 8.1 (d, 1H), 7.7 (s, 1H), 7.6 (d, 1H), 7.3 (d, 1H), 7.2 (d, 1H), 5.0 (q, 1H), 2.9 (m, 4H), 2.8 (m, 1H), 2.4 (m, 4H), 2.0 (m, 2H), 1.8 (m, 2H), 1.6 (m, 2H).

### Example 12

### 6-(3-Cyclobutyl-2,3,4,5-tetrahydro-1H-benzo[d]azepin-7-yl)-2-ethyl-4,5-dihydro-2H-pyridazin-3-one

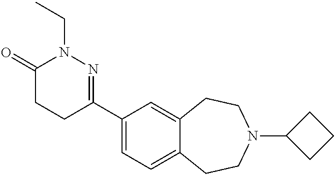

Synthesized via Scheme 2: mp=97-98° C., MS m/z=326 (M+1), HNMR (DMSO): 7.55 (s, 1H), 7.5 (d, 1H), 7.15 (d, 1H), 3.8 (q, 2H), 2.9 (q, 2H), 2.8 (m, 4H), 2.7 (m, 1H), 2.5 (t, 2H), 2.3 (m, 4H), 2.0 (m, 2H), 1.8 (m, 2H), 1.6 (m, 2H), 1.2 (t, 2H).

### Example 13

### 2-Butyl-6-(3-cyclobutyl-2,3,4,5-tetrahydro-1H-benzo[d]azepin-7-yl)-4,5-dihydro-2H-pyridazin-3-one

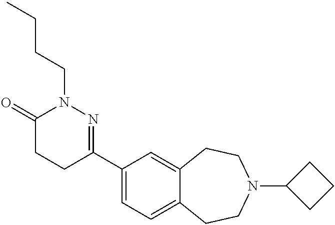

Synthesized via Scheme 2: mp=104-105° C., MS m/z=354 (M+1), HNMR (DMSO): 7.55 (s, 1H), 7.5 (d, 1H), 7.2 (d, 1H), 3.7 (t, 2H), 2.9 (q, 2H), 2.8 (m, 3H), 2.7 (m, 1H), 2.45 (t, 2H), 2.3 (m, 4H), 2.0 (m, 2H), 1.8 (m, 2H), 1.6 (m, 4H), 1.3 (m, 2H), 0.9 (t, 3H).

### Example 14

### 2-Butyl-6-(3-cyclobutyl-2,3,4,5-tetrahydro-1H-benzo[d]azepin-7-yl)-2H-pyridazin-3-one

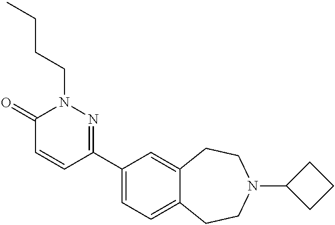

Synthesized via Scheme 1: mp=88-89° C., MS m/z=352 (M+1), HNMR (DMSO): 8.0 (d, 1H), 7.65 (s, 1H), 7.55 (d, 1H), 7.2 (d, 1H), 7.0 (d, 1H), 4.1 (t, 2H), 2.9 (m, 4H), 2.8 (m, 1H), 2.4 (m, 4H), 2.0 (m, 2H), 1.8 (m, 4H), 1.6 (m, 2H), 1.3 (m, 2H), 0.9 (t, 3H).

### Example 15

### 6-(3-Cyclobutyl-2,3,4,5-tetrahydro-1H-benzo[d]azepin-7-yl)-2-isobutyl-4,5-dihydro-2H-pyridazin-3-one

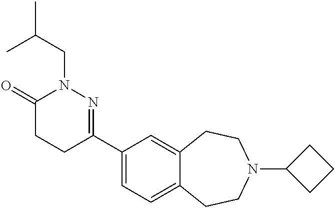

Synthesized via Scheme 2: mp=134-135° C., MS m/z=354 (M+1), HNMR (DMSO): 7.55 (s, 1H), 7.5 (d, 1H), 7.2 (d, 1H), 3.45 (d, 2H), 2.9 (t, 2H), 2.8 (m, 4H), 2.7 (m, 1H), 2.35 (m, 4H), 2.0 (m, 4H), 1.7 (m, 2H), 1.5 (m, 2H), 0.9 (d, 6H).

### Example 16

### 6-(3-Cyclobutyl-2,3,4,5-tetrahydro-1H-benzo[d]azepin-7-yl)-2-isobutyl-2H-pyridazin-3-one

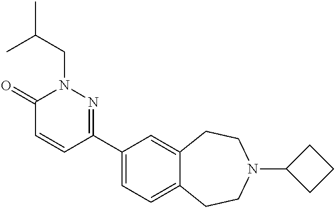

Synthesized via Scheme 1: mp=245-246° C., MS=352 (M+1), HNMR (DMSO): 8.0 (d, 1H), 7.65 (s, 1H), 7.6 (d, 1H), 7.2 (d, 1H), 7.0 (d, 1H), 4.0 (d, 2H), 2.9 (m, 4H), 2.8 (m, 1H), 2.3 (m, 4H), 2.2 (m, 1H), 2.0 (m, 2H), 1.8 (m, 2H), 1.6 (m, 2H), 0.9 (d, 6H).

### Example 17

### 6-(3-Cyclobutyl-2,3,4,5-tetrahydro-1H-benzo[d]azepin-7-yl)-2-(2-hydroxy-ethyl)-4,5-dihydro-2H-pyridazin-3-one

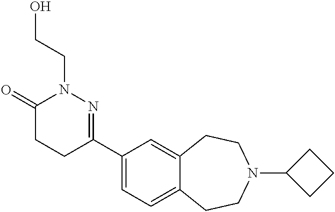

Synthesized via Scheme 2: mp=141-142° C., MS m/z=342 (M+1), HNMR (DMSO): 7.55 (s, 1H), 7.50 (d, 1H), 7.2 (d, 1H), 4.7 (t, 1H), 3.8 (m, 2H), 3.6 (m, 2H), 2.9 (t, 2H), 2.8 (m, 4H), 2.7 (m, 1H), 2.3 (m, 4H), 2.0 (m, 2H), 1.8 (m, 2H), 1.6 (m, 2H).

### Example 18

### 6-(3-Cyclobutyl-2,3,4,5-tetrahydro-1H-benzo[d]azepin-7-yl)-2-(2-hydroxy-ethyl)-2H-pyridazin-3-one

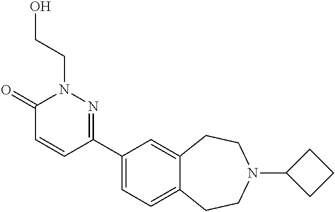

Synthesized via Scheme 1: mp=157-158° C., MS m/z/=340 (M+1), HNMR (DMSO): 8.0 (d, 1H), 7.65 (s, 1H), 7.6 (d, 1H), 7.2 (d, 1H), 7.0 (d, 1H), 4.85 (t, 1H), 4.2 (t, 2H), 3.8 (m, 2H), 2.9 (m, 4H), 2.8 (m, 1H), 2.3 (m, 4H), 2.0 (m, 2H), 1.8 (m, 2H), 1.6 (m, 2H).

### Example 19

### 2-Cyclobutyl-6-(3-cyclobutyl-2,3,4,5-tetrahydro-1H-benzo[d]azepin-7-yl)-4,5-dihydro-2H-pyridazin-3-one

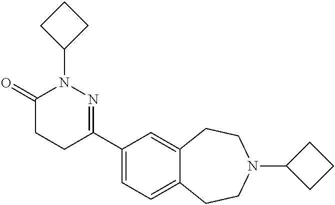

Synthesized via Scheme 2: mp=126-128° C., MS m/z=352 (M+1), HNMR (CDCl₃): 7.6 (m, 2H), 7.15 (d, 1H), 5.3 (m, 1H), 2.9 (m, 6H), 2.8 (m, 1H), 2.5 (m, 8H), 2.2 (m, 2H), 2.1 (m, 2H), 1.9 (m, 2H), 1.7 (m, 4H).

### Example 20

### 2-Methyl-6-(2,3,4,5-tetrahydro-1H-benzo[d]azepin-7-yl)-2H-pyridazin-3-one

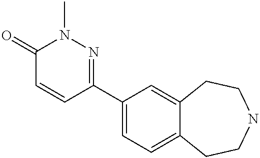

### Step 1: Synthesis of 2-Methyl-6-[3-(2,2,2-trifluoro-acetyl)-2,3,4,5-tetrahydro-1H-benzo[d]azepin-7-yl]-4,5-dihydro-2H-pyridazin-3-one

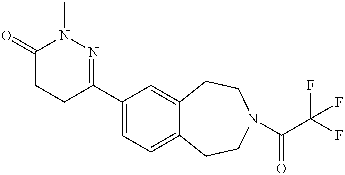

A solution of 4-oxo-4-[3-(2,2,2-trifluoro-acetyl)-2,3,4,5-tetrahydro-1H-benzo[d]azepin-7-yl]-butyric acid methyl ester (2.23 g, 6.2 mmol) and methyl hydrazine (0.287 g, 6.2 mmol) in isopropanol (20 mL) was heated at 120° C. for 20 h. Low volatiles were evaporated under vacuum and partitioned between ethyl acetate and saturated aqueous sodium bicarbonate solution. The aqueous layer was extracted twice with ethyl acetate and the combined organics was washed with brine, dried (Na₂SO₄), filtered and concentrated to give a crude product. The crude product was purified by Biotage chromatography using 2% methanol in methylene chloride to produce 2-methyl-6-[3-(2,2,2-trifluoro-acetyl)-2,3,4,5-tetrahydro-1H-benzo[d]azepin-7-yl]-4,5-dihydro-2H-pyridazin-3-one (2.0 g, 91%), MS m/z=354 (M+1).

### Step 2: Synthesis of 2-Methyl-6-[3-(2,2,2-trifluoro-acetyl)-2,3,4,5-tetrahydro-1H-benzo[d]azepin-7-yl]-2H-pyridazin-3-one

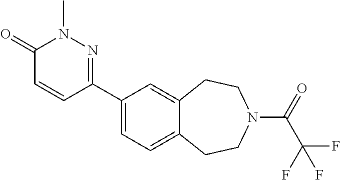

A mixture of 2-methyl-6-[3-(2,2,2-trifluoro-acetyl)-2,3,4,5-tetrahydro-1H-benzo[d]azepin-7-yl]-4,5-dihydro-2H-pyridazin-3-one (1.02 g, 2.8 mmol) and selenium dioxide (1.44 g, 12.9 mmol) in acetic acid (12 ml) was heated at 125° C. for 4 h. The reaction mixture was diluted with methylene chloride and filtered over celite then washed with 1% methanol in methylene chloride. The filtrate was concentrated under vacuum and the residue was dissolved in methylene chloride then washed with saturated aqueous sodium bicarbonate solution, brine, dried (Na₂SO₄), filtered and concentrated to provide a product (0.94 g, 93%). The analysis of the product showed a major compound (2-methyl-6-[3-(2,2,2-trifluoro-acetyl-2,3,4,5-tetrahydro-1H-benzo[d]azepin-7-yl]-2H-pyridazin-3-one), MS m/z=352 (M+1) and a minor compound (2-methyl-6-(2,3,4,5-tetrahydro-1H-benzo[d]azepin-7-yl)-2H-pyridazin-3-one), MS m/z=256 (M+H). The crude mixture was used for the next reaction without further purification.

### Step 3: Synthesis of Example 20

A mixture of the product from step 2 (2-methyl-6-[3-(2,2,2-trifluoro-acetyl-2,3,4,5-tetrahydro-1H-benzo[d]azepin-7-yl]-2H-pyridazin-3-one) (0.945 g, 2.6 mmol) and potassium carbonate (1.12 g, 8.1 mmol) in a mixture of tetrahydrofuran (2 mL) and methanol (10 mL) was stirred at room temperature for 3 h. The reaction mixture was evaporated under vacuum then partitioned between saturated aqueous sodium bicarbonate solution and 1% methanol in methylene chloride. The aqueous phase was extracted twice with 1% methanol in methylene chloride and the combined organics was washed with brine, dried (Na₂SO₄), filtered and concentrated to afford a crude product. The crude product was purified by Biotage chromatography using 4% methanol in methylene chloride to 10% methanol containing 3 mL of ammonium hydroxide in methylene chloride to afford a pure product. The pure product was dissolved in methylene chloride and washed with saturated aqueous sodium bicarbonate solution, brine, dried (Na₂SO₄), filtered and concentrated to produce 2-methyl-6-(2,3,4,5-tetrahydro-1H-benzo[d]azepin-7-yl)-2H-pyridazin-3-one, (430 mg, 62%), mp 183-185° C. (methylene chloride and methanol), MS m/z=256 (M+1), ¹H NMR (400 MHz, CDCl₃, δ): 3.87 (s, 3H), 7.00 (d, J=9.64 Hz, 1H), 7.19 (d, J=7.78 Hz, 1H), 7.46-7.57 (m, 1H), 7.54-7.57 (m, 1H), 7.66 (d, J=9.64 Hz, 1H).

### Example 21

### 6-(3-Cyclobutyl-2,3,4,5-tetrahydro-1H-benzo[d]azepin-7-yl)-2-methyl-2H-pyridazin-3-one

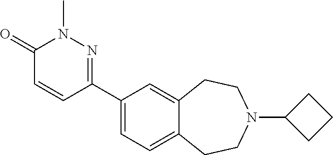

A solution of 2-methyl-6-(2,3,4,5-tetrahydro-1H-benzo[d]azepin-7-yl)-2H-pyridazin-3-one (0.430 g, 1.6 mmol) in methylene chloride (6 mL) was added acetic acid (0.153 mL). After 10 min at room temperature, cyclobutanone (0.177 g, 2.52 mmol) was added dropwise and further stirred at room temperature for 1 h. Sodium triacetoxyborohydride (0.536 g, 2.52 mmol) was added in portions at room temperature and further stirred at room temperature for 2.5 h then quenched with 2M sodium carbonate solution. The aqueous layer was extracted twice with methylene chloride and the combined organics was washed with brine, dried (Na₂SO₄), filtered and concentrated to provide a crude product. The crude product was purified by Biotage chromatography using 3 to 10% methanol in methylene chloride to 10% methanol containing 3 mL of ammonium hydroxide in methylene chloride to obtain a product. The product was dissolved in methylene chloride and washed with saturated aqueous sodium bicarbonate solution, brine, dried (Na₂SO₄), filtered and concentrated to afford a pure product. The pure product was crystallized from a mixture of ethanol, ethyl acetate, ether and hexane followed by drying in a ChemDry at 75° C. for 22 h to produce 6-(3-cyclobutyl-2,3,4,5-tetrahydro-1H-benzo[d]azepin-7-yl)-2-methyl-2H-pyridazin-3-one (440 g, 84%), mp 287-288° C. (ethanol, ethyl acetate, ether and hexane), MS m/z=310 (M+1), ¹H NMR (400 MHz, DMSO-d₆, δ): 1.55-1.80 (m, 2H), 2.15-2.25 (m, 2H), 2.37-2.52 (m, 2H), 2.70-2.87 (m, 2H), 2.98-3.15 (m, 2H), 3.37-3.57 (m, 4H), 3.58-3.70 (m, 2H), 3.73 (s, 3H), 7.05 (d, J=9.75 Hz, 1H), 7.34 (d, J=7.83 Hz, 1H), 7.71 (d, J=7.86 Hz, 1H), 7.76 (br s, 1H), 8.04 (d, J=9.73 Hz, 1H).

### Example 22

### 6-(3-Isopropyl-2,3,4,5-tetrahydro-1H-benzo[d]azepin-7-yl)-2-methyl-2H-pyridazin-3-one. HCl

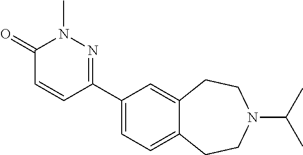

Example 22 was prepared using the method for Example 21. mp=254-256° C.; MS m/z=298 (M+1); ¹H NMR (400 MHz, DMSO-d₆, δ): 1.275 (d, J=6.5 Hz, 6H), 2.90-3.14 (m, 4H), 3.49-3.68 (m, 4H), 3.73 (s, 3H), 7.03 (d, J=9.73 Hz, 1H), 7.34 (d, J=7.86 Hz, 1H), 7.71 (d, J=7.86 Hz, 1H), 7.77 (br s, 1H), 8.035 (d, J=9.69 Hz, 1H).

### Example 23

### 6-(3-Cyclopentyl-2,3,4,5-tetrahydro-1H-benzo[d]azepin-7-yl)-2-methyl-2H-pyridazin-3-one. HCl

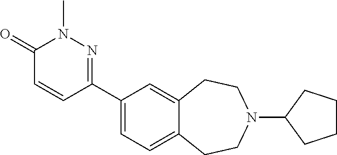

Example 23 was prepared using the method for Example 21. mp=239-240° C.; MS m/z=324 (M+1); ¹H NMR (400 MHz, DMSO-d₆, δ): 1.50-1.64 (m, 2H), 1.66-1.89 (m, 4H), 1.96-2.10 (m, 2H), 2.95-3.20 (m, 4H), 3.25-3.42 (m, 3H), 3.61-3.78 (m, 2H), 3.74 (s, 3H), 7.05 (d, J=9.78 Hz, 1H), 7.34 (d, J=7.72 Hz, 1H), 7.72 (d, J=7.98 Hz, 1H), 7.76 (br s, 1H), 8.04 (d, J=9.66 Hz, 1H).

### Example 24

### 2-(4-Methanesulfonyl-phenyl)-6-(2,3,4,5-tetrahydro-1H-benzo[d]azepin-7-yl)-2H-pyridazin-3-one

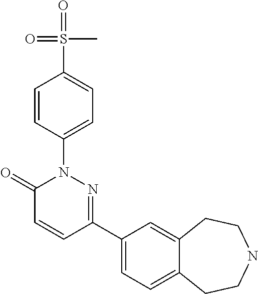

### Step 1: Synthesis of 2-(4-methanesulfonyl-phenyl)-6-[3-(2,2,2-trifluoro-acetyl)-2,3,4,5-tetrahydro-1H-benzo[d]azepin-7-yl]-4,5-dihydro-2H-pyridazin-3-one

A mixture of 4-oxo-4-[3-(2,2,2-trifluoro-acetyl)-2,3,4,5-tetrahydro-1H-benzo[d]azepin-7-yl]-butyric acid methyl ester (2.5 g, 7.00 mmol), (4-methylsulfonyl-phenyl)-hydrazine.HCl (1.86 g, 8.34 mmol) and diisopropylamine (1.37 mL, 8.34 mmol) in isopropanol (39 mL) was heated at 120° C. for 2.5 days followed by microwave irradiation (150° C., 200 psi) for 85 min. The reaction mixture was concentrated under vacuum and dissolved in 1% methanol in methylene chloride then washed with saturated aqueous sodium bicarbonate solution, brine, dried (Na₂SO₄), filtered and concentrated to obtain a crude product. The crude product was purified by Biotage chromatography to produce 2-(4-methanesulfonyl-phenyl)-6-[3-(2,2,2-trifluoro-acetyl)-2,3,4,5-tetrahydro-1H-benzo[d]azepin-7-yl]-4,5-dihydro-2H-pyridazin-3-one (2.5 g, 72%), MS m/z=494 (M+1).

### Step 2: Synthesis of 2-(4-Methanesulfonyl-phenyl)-6-[3-(2,2,2-trifluoro-acetyl)-2,3,4,5-tetrahydro-1H-benzo[d]azepin-7-yl]-2H-pyridazin-3-one

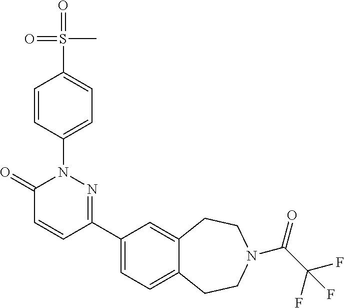

A mixture of 2-(4-methanesulfonyl-phenyl)-6-[3-(2,2,2-trifluoro-acetyl)-2,3,4,5-tetrahydro-1H-benzo[d]azepin-7-yl]-4,5-dihydro-2H-pyridazin-3-one (2.5 g, 5.07 mmol) and selenium dioxide (2.95 g, 26.57 mmol) in acetic acid (25 mL) was heated at 125° C. for 4.5 h. The reaction mixture was diluted with 1% methanol in methylene chloride and filtered over celite then washed with methylene chloride. The reaction mixture was concentrated under vacuum and the crude residue was dissolved in methylene chloride then washed with saturated aqueous sodium bicarbonate solution, brine, dried (Na₂SO₄), filtered and concentrated to obtain a crude product. The crude product was purified by Biotage chromatography using 2% methanol in methylene chloride to produce 2-(4-methanesulfonyl-phenyl)-6-[3-(2,2,2-trifluoro-acetyl)-2,3,4,5-tetrahydro-1H-benzo[d]azepin-7-yl]-2H-pyridazin-3-one (1.4 g, 58%), MS m/z=492 (M+1).

### Synthesis of Example 24

A mixture of 2-(4-methanesulfonyl-phenyl)-6-[3-(2,2,2-trifluoro-acetyl)-2,3,4,5-tetrahydro-1H-benzo[d]azepin-7-yl]-2H-pyridazin-3-one (1.4 g, 2.85 mmol) and potassium carbonate (1.5 g, 10.86 mmol) in a mixture of methanol (15 mL) and tetrahydrofuran (10 mL) was stirred at room temperature for 2 h. The reaction mixture was concentrated under vacuum and dissolved in methylene chloride then washed with saturated aqueous sodium bicarbonate solution, brine, dried (Na₂SO₄), filtered and concentrated to obtain a crude product. The crude product was purified by Biotage chromatography using 2% to 10% methanol in methylene chloride to 10% methanol containing 2 mL ammonium hydroxide in methylene chloride to give a pure product. The pure product was dissolved in methylene chloride and washed with saturated aqueous sodium bicarbonate solution, brine, dried (Na₂SO₄), filtered and concentrated to produce 2-(4-methanesulfonyl-phenyl)-6-(2,3,4,5-tetrahydro-1H-benzo[d]azepin-7-yl)-2H-pyridazin-3-one, (0.48 g, 42%), MS m/z=396 (M+1).

### Example 25

### 6-(3-Cyclobutyl-2,3,4,5-tetrahydro-1H-benzo[d]azepin-7-yl)-2-(4-methanesulfonyl-phenyl)-2H-pyridazin-3-one

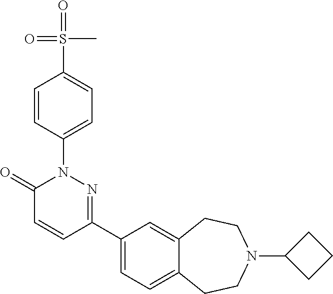

A solution of 2-(4-methanesulfonyl-phenyl)-6-(2,3,4,5-tetrahydro-1H-benzo[d]azepin-7-yl)-2H-pyridazin-3-one (0.480 g, 1.21 mmol) in methylene chloride (7 mL) was added acetic acid (0.175 mL). After stirring for 10 min at room temperature, cyclobutanone (0.137 g, 1.81 mmol) was added dropwise and further stirred at room temperature for 1 h. Sodium triacetoxyborohydride (0.386 g, 1.82 mmol) was added in portions at room temperature and further stirred at room temperature for 20 h then quenched with 2M aqueous sodium carbonate solution. The aqueous layer was extracted twice with methylene chloride and the combined organics was washed with brine, dried (Na₂SO₄), filtered and concentrated to provide a crude product. The crude product was purified by Biotage chromatography using 4 to 10% methanol in methylene chloride to 10% methanol containing 3 mL ammonium hydroxide in methylene chloride to obtain a pure product. The pure product was dissolved in ethyl acetate and washed with saturated aqueous sodium bicarbonate solution, brine, dried (Na₂SO₄), filtered and concentrated to give a product. The product was triturated from ether followed by drying to produce 6-(3-cyclobutyl-2,3,4,5-tetrahydro-1H-benzo[d]azepin-7-yl)-2-(4-methanesulfonyl-phenyl)-2H-pyridazin-3-one (0.150 g, 27%), mp 225° C. (ether), MS m/z=450 (M+1), ¹H NMR (400 MHz, DMSO-d₆, δ): 1.60-1.80 (m, 2H), 1.87-2.02 (m, 2H), 2.06-2.17 (m, 2H), 2.42-2.58 (m, 4H), 2.78-2.88 (m, 1H), 2.95-3.07 (m, 4H), 3.11 (s, 3H), 7.16 (d, J=9.75 Hz, 1H), 7.23 (d, J=7.69 Hz, 1H), 7.55-7.61 (m, 2H), 7.785 (d, J=9.73 Hz, 1H), 8.04 (d, J=7.54 Hz, 2H), 8.09 (d, J=8.73 Hz, 2H).

### Synthesis of pyridyl-ether examples

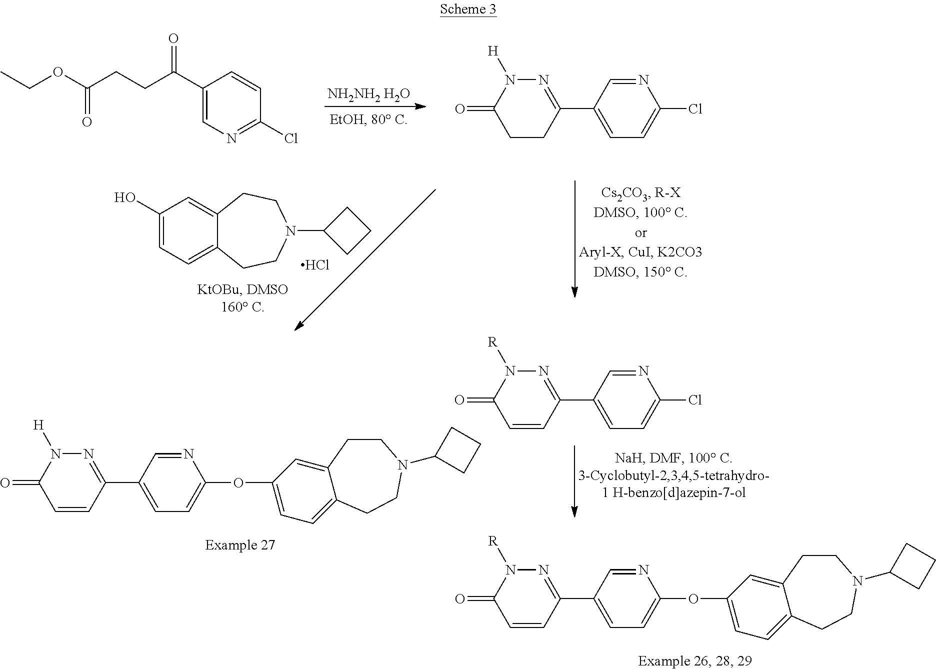

### Example 26

### 6-[6-(3-Cyclobutyl-2,3,4,5-tetrahydro-1H-benzo[d]azepin-7-yloxy)-pyridin-3-yl]-2-methyl-2H-pyridazin-3-one

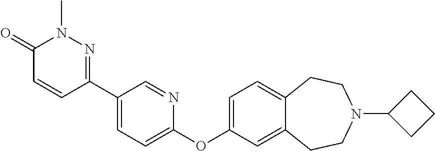

### Step 1: Synthesis of 6-(6-Chloro-pyridin-3-yl)-4,5-dihydro-2H-pyridazin-3-one

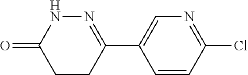

To ethyl-4-(4-chloro-3-pyridyl)-4-oxobutyrate (5 g, 20.7 mmol) in ethanol (30 mL) was added hydrazine monohydrate (964 μM, 31 mmol). After overnight stirring at 80° C., the reaction was concentrated to half of the volume and the resulting yellow solid was filtered off and dried to give 3.4 g product (77%); MS m/z=210 (M+1).

### Step 2: Synthesis of 6-(6-Chloro-pyridin-3-yl)-2-methyl-2H-pyridazin-3-one

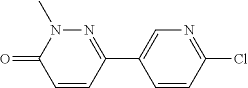

To 6-(6-chloro-pyridin-3-yl)-4,5-dihydro-2H-pyridazin-3-one (1.03 g, 4.93 mmol) in DMSO (40 mL) was added iodomethane (460 μL, 7.4 mmol) and cesium carbonate (3.2 g, 9.86 mmol). After overnight stirring at 100° C. open to air, the reaction was cooled, diluted with dichloromethane, washed with water/brine, dried over sodium sulfate, and concentrated under vacuum to obtain 835 mg (79%); MS m/z=222 (M+1).

### Step 3: Synthesis of Example 26

To 3-cyclobutyl-2,3,4,5-tetrahydro-1H-benzo[d]azepin-7-ol hydrochloride (688 mg, 2.71 mmol) in DMF (7 mL) was added sodium hydride (60% in mineral oil) (217 mg, 5.43 mmol) at 25° C. The reaction was stirred for 1 h before adding 6-(6-chloro-pyridin-3-yl)-2-methyl-2H-pyridazin-3-one (400 mg, 1.81 mmol). After overnight stirring at 100° C., the reaction was cooled, filtered through celite, partitioned between dichloromethane/1N sodium carbonate, washed with water/brine, dried over sodium sulfate, and concentrated. The product was purified using Prep TLC plates (9:1 dichloromethane:methanol) to obtain 262 mg (36%); mp 134-137° C.; MS m/z=403 (M+1); ¹HNMR δ (DMSO-d₆) 8.64 (s, 1H), 8.30 (dd, 1H, J=1.1, 8.6 Hz), 8.05 (d, 1H, J=9.7 Hz), 7.16 (d, 1H, J=8.0 Hz), 7.10 (m, 2H), 6.93 (s, 1H), 6.88 (d, 1H, J=8.0 Hz), 3.73 (s, 3H), 2.84 (b m, 5H), 2.36 (b m, 4H), 2.00 (b m, 2H), 1.78 (b m, 2H), 1.62 (b m, 2H).

### Example 27

### 6-[6-(3-Cyclobutyl-2,3,4,5-tetrahydro-1H-benzo[d]azepin-7-yloxy)-pyridin-3-yl]-2H-pyridazin-3-one

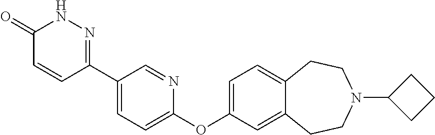

To 6-(6-chloro-pyridin-3-yl)-4,5-dihydro-2H-pyridazin-3-one (400 mg, 1.91 mmol) in DMSO (20 mL) was added 1M KtOBu (5.73 mL, 5.73 mmol), followed by 3-cyclobutyl-2,3,4,5-tetrahydro-1H-benzo[d]azepin-7-ol hydrochloride (581 mg, 2.3 mmol). After overnight stirring at 160° C. open to air, the reaction was partitioned between chloroform/water, washed with brine, dried over sodium sulfate, and concentrated. The product was purified using Prep TLC plates (9:1 dichloromethane:methanol) to obtain 20 mg; mp 215-220° C.; MS m/z=389 (M+1); ¹HNMR δ (DMSO-d₆) 13.22 (s, 1H), 8.61 (s, 1H), 8.28 (dd, 1H, J=1.3, 8.7 Hz), 8.04 (d, 1H, J=9.9 Hz), 7.17 (d, 1H, J=8.0 Hz), 7.09 (d, 1H, J=8.7 Hz), 7.02 (d, 1H, J=9.9 Hz), 6.93 (s, 1H), 6.89 (d, 1H, J=7.6 Hz), 2.85 (br m, 5H), 2.37 (br m, 4H), 2.01 (br m, 2H), 1.80 (br m, 2H), 1.61 (br m, 2H).

### Example 28

### 6-[6-(3-Cyclobutyl-2,3,4,5-tetrahydro-1H-benzo[d]azepin-7-yloxy)-pyridin-3-yl]-2-pyridin-2-yl-2H-pyridazin-3-one

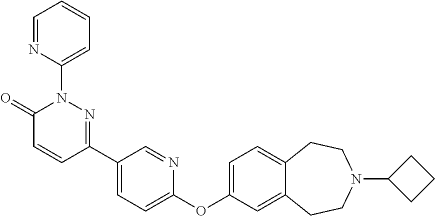

### Step 1: Synthesis of 6-(6-Chloro-pyridin-3-yl)-2-pyridin-2-yl-2H-pyridazin-3-one

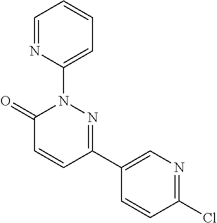

To 6-(6-chloro-pyridin-3-yl)-4,5-dihydro-2H-pyridazin-3-one (700 mg, 3.35 mmol) in DMSO (20 mL) was added 2-bromopyridine (653 μL, 6.7 mmol), potassium carbonate (1.4 g, 10.05 mmol), and copper(I) iodide (64 mg, 0.34 mmol). After overnight stirring at 150° C. open to air, the reaction was filtered through celite, partitioned between chloroform/1N sodium carbonate, washed with water/brine several times, dried over sodium sulfate, and concentrated under vacuum to obtain 804 mg crude product (85%); MS m/z=285 (M+1).

### Step 2: Synthesis of Example 28

To 3-cyclobutyl-2,3,4,5-tetrahydro-1H-benzo[d]azepin-7-ol hydrochloride (861 mg, 3.4 mmol) in DMF (10 mL) was added sodium hydride (60% in mineral oil) (226 mg, 5.7 mmol) at 25° C. The reaction was stirred for 30 min. before adding 6-(6-chloro-pyridin-3-yl)-2-pyridin-2-yl-2H-pyridazin-3-one (804 mg, 2.83 mmol) in DMF (2 mL). After overnight stirring at 100° C., the reaction was filtered through celite, partitioned between dichloromethane/1N sodium carbonate (poor solubility), washed with water/brine several times, dried over sodium sulfate, and concentrated. The product was purified using Prep. TLC plates (9:1 dichloromethane:methanol) to obtain 100 mg (<10%); mp 178-182° C.; MS m/z 466 (M+H); ¹HNMR δ (DMSO-d₆) 8.66 (m, 2H), 8.28 (dd, 1H, J=2.5, 8.7 Hz), 8.19 (d, 1H, J=9.9 Hz), 8.07 (m, 1H), 7.70 (d, 1H, J=8.0 Hz), 7.57 (m, 1H), 7.25 (d, 1H, J=9.9 Hz), 7.16 (d, 1H, J=8.0 Hz), 7.09 (d, 1H, J=8.7 Hz), 6.93 (s, 1H), 6.89 (d, 1H, J=8.0 Hz), 2.84 (br m, 5H), 2.36 (br m, 4H), 2.00 (br m, 2H), 1.78 (br m, 2H), 1.57 (br m, 2H).

### Example 29

### 6-[6-(3-Cyclobutyl-2,3,4,5-tetrahydro-1H-benzo[d]azepin-7-yloxy)-pyridin-3-yl]-2-isopropyl-2H-pyridazin-3-one

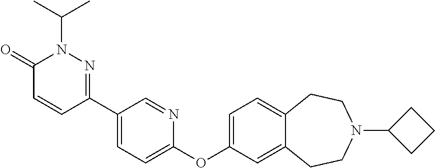

### Step 1: Synthesis of 6-(6-Chloro-pyridin-3-yl)-2-isopropyl-1,6-dihydro-2H-pyridazin-3-one

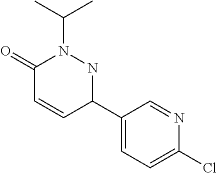

This compound was prepared by following Step 2 in Example 26. MS m/z=250 (M+1).

### Step 2: Synthesis of Example 29

To 3-cyclobutyl-2,3,4,5-tetrahydro-1H-benzo[d]azepin-7-ol hydrochloride (777 mg, 3.1 mmol) in DMF (10 mL) was added sodium hydride (60% in mineral oil) (245 mg, 6.1 mmol) at 25° C. The reaction was stirred for 1 h before adding 6-(6-chloro-pyridin-3-yl)-2-isopropyl-2H-pyridazin-3-one (509 mg, 2.04 mmol). After overnight stirring at 110° C., the reaction was filtered through celite, partitioned between dichloromethane/1N sodium carbonate, washed with water/brine, dried over sodium sulfate, and concentrated. The product was purified using Prep TLC plates (9:1 dichloromethane:methanol) and dried under vacuum to obtain 184 mg (21%); mp 70-73° C.; MS m/z 431 (M+H); ¹HNMR δ (DMSO-d₆) 8.67 (s, 1H), 8.35 (dd, 1H, J=1.4, 8.6 Hz), 8.03 (d, 1H, J=9.7 Hz), 7.16 (m, 2H), 7.04 (m, 1H), 6.93 (s, 1H), 6.89 (d, 1H, J=8.0 Hz), 5.21 (m, 1H), 2.84 (br m, 5H), 2.36 (br m, 4H), 2.02 (br m, 2H), 1.80 (br m, 2H), 1.62 (br m, 2H), 1.34 (d, 6H, J=6.6 Hz).

### Example 30

### 4-Chloro-5-(3-cyclobutyl-2,3,4,5-tetrahydro-1H-benzo[d]azepin-7-yloxy)-2-methyl-2H-pyridazin-3-one

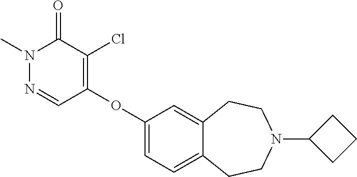

### General Synthesis of Example 30

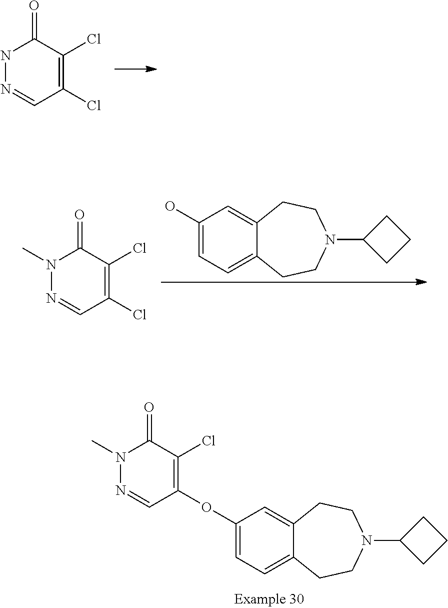

### Step 1

To a round-bottom flask was added 4,5-dichloro-2H-pyridazin-3-one (10.0 g, 60.6 mmol), potassium carbonate (16.8 g, 121.2 mmol), iodomethane (4.0 mL, 64.3 mmol), and acetonitrile (80 mL). The reaction mixture was heated at reflux for 12 h and was cooled to room temperature. The reaction was filtered and the filtrate was concentrated. The residue was purified by column chromatography (CH₂Cl₂) to give 8.21 g (76%) of 4,5-dichloro-2-methyl-2H-pyridazin-3-one. ¹HNMR (400 MHz, DMSO-d₆) δ 8.18 (s, 1H), 3.70 (s, 3H); MS (m/z)=179 (M+1).

### Step 2

To a Carousel reaction tube was added 4,5-dichloro-2-methyl-2H-pyridazin-3-one (200 mg, 1.12 mmol), 3-cyclobutyl-2,3,4,5-tetrahydro-1H-benzo[d]azepin-7-ol hydrochloride acid (299 mg, 1.18 mmol), potassium carbonate (464 mg, 3.36 mmol), and DMF (5 mL). The reaction was heated at 130° C. for 23 h and was cooled to room temperature. The reaction was filtered through a pad of Celite and eluted with DMF. The filtrate was concentrated and the residue was purified by column chromatography (2% MeOH/CH₂Cl₂) to give 214 mg (53%) of 4-chloro-5-(3-cyclobutyl-2,3,4,5-tetrahydro-1H-benzo[d]azepin-7-yloxy)-2-methyl-2H-pyridazin-3-one. ¹HNMR (400 MHz, DMSO-d₆) δ 7.68 (s, 1H), 7.18 (m, 1H), 7.00 (s, 1H), 6.93 (m, 1H), 3.69 (s, 3H), 2.83 (m, 4H), 2.76 (m, 1H), 2.34 (s, broad, 4H), 2.00 (m, 2H), 1.78 (m, 2H), 1.59 (m, 2H); MS (m/z)=360 (M+1).

### Example 31

### 4-Bromo-5-(3-cyclobutyl-2,3,4,5-tetrahydro-1H-benzo[d]azepin-7-yloxy)-2-methyl-2H-pyridazin-3-one

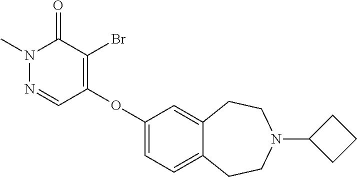

Example 31 was synthesized using methods similar to Example 30.

¹H NMR (400 MHz, DMSO-d₆) δ 7.57 (s, 1H), 7.18 (m, 1H), 6.98 (s, 1H), 6.92 (m, 1H), 3.69 (s, 3H), 2.83 (m, 4H), 2.76 (m, 1H), 2.35 (s, broad, 4H), 1.99 (m, 2H), 1.76 (m, 2H), 1.58 (m, 2H); MS (m/z)=404 (M+).

### Example 32

### 4-Bromo-5-(3-cyclopentyl-2,3,4,5-tetrahydro-1H-benzo[d]azepin-7-yloxy)-2-methyl-2H-pyridazin-3-one

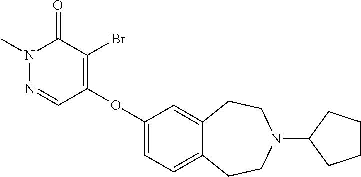

Example 32 was synthesized using methods similar to Example 30.

¹H NMR (400 MHz, DMSO-d₆) δ 7.57 (m, 1H), 7.18 (m, 1H), 7.00 (s, 1H), 6.92 (m, 1H), 3.70 (s, 3H), 2.86 (s, broad, 5H), 2.61 (s, broad, 4H), 1.77 (s, broad, 2H), 1.64-1.32 (m, 6H); MS (m/z)=418 (M+).

### Example 33

### 4-Chloro-5-(3-cyclobutyl-2,3,4,5-tetrahydro-1H-benzo[d]azepin-7-yloxy)-2-pyridin-2-yl-2H-pyridazin-3-one

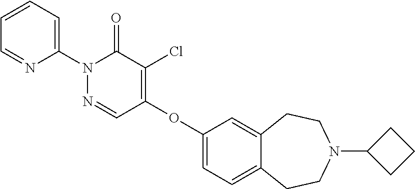

### General Synthesis of Example 33

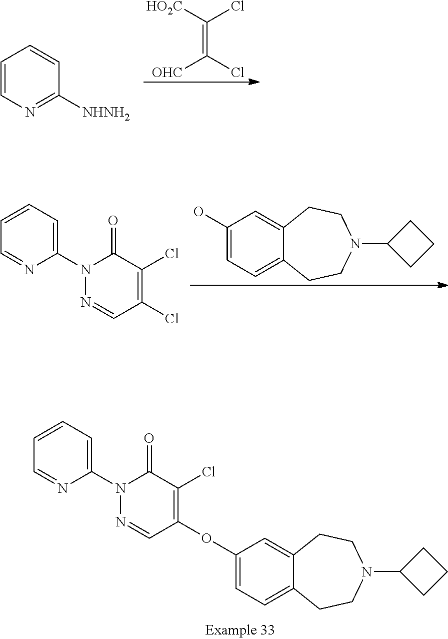

### Step 1

To a stirring mixture of pyridine-2-yl-hydrazine (5.00 g, 45.8 mmol) and mucochloric acid (7.74 g, 45.8 mmol) in ethanol (150 mL) was added concentrated HCl solution (2 mL). The reaction was heated at reflux for 2 h and was cooled to room temperature. The precipitation was collected by filtration and was washed with Et₂O, dried to give 7.51 g of 4,5-dichloro-2-pyridin-2-yl-2H-pyridazin-3-one. The filtrate was concentrated and was purified by column chromatography (2% MeOH/CH₂Cl₂) to give another 1.22 g of 4,5-dichloro-2-pyridin-2-yl-2H-pyridazin-3-one. The two fractions was combined to give a total 8.73 g (79%) of 4,5-dichloro-2-pyridin-2-yl-2H-pyridazin-3-one. ¹H NMR (400 MHz, DMSO-d₆) δ 8.62 (m, 1H), 8.35 (s, 1H), 8.07 (m, 1H), 7.64 (m, 1H), 7.57 (m, 1H); MS (m/z)=242 (M+).

### Step 2

To a Carousel reaction tube was added 4,5-dichloro-2-pyridin-2-yl-2H-pyridazin-3-one (200 mg, 0.826 mmol), 3-cyclobutyl-2,3,4,5-tetrahydro-1H-benzo[d]azepin-7-ol hydrochloride acid (220 mg, 0.867 mmol), potassium carbonate (343 mg, 2.48 mmol), and DMF (5 mL). The reaction was heated at 130° C. for 16 h and was cooled to room temperature. The reaction was filtered through a pad of Celite and the filtrate was concentrated. Purification by column chromatography (3% MeOH/CH₂Cl₂) to give 103 mg (29%) of 4-chloro-5-(3-cyclobutyl-2,3,4,5-tetrahydro-1H-benzo[d]azepin-7-yloxy)-2-pyridin-2-yl-2H-pyridazin-3-one. ¹H NMR (400 MHz, DMSO-d₆) δ 8.61 (m, 1H), 8.06 (m, 1H), 7.85 (s, 1H), 7.62 (m, 1H), 7.57 (m, 1H), 7.25 (m, 1H), 7.13 (s, 1H), 7.06 (m, 1H), 4.08 (s, 1H), 2.91 (m, 6H), 2.55 (m, 2H), 2.04 (m, 2H), 1.88 (m, 2H), 1.60 (m, 2H); MS (m/z)=423 (M+1).

### Example 34

### 4-Chloro-5-(3-cyclobutyl-2,3,4,5-tetrahydro-1H-benzo[d]azepin-7-yloxy)-2-(2,2,2-trifluoro-ethyl)-2H-pyridazin-3-one

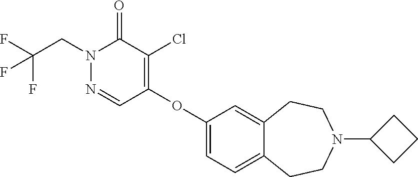

This compound was synthesized by the methods described for Example 33.

¹H NMR (400 MHz, DMSO-d₆) δ 7.81 (s, 1H), 7.20 (m, 1H), 7.06 (s, 1H), 6.99 (m, 1H), 4.99 (m, 2H), 2.84 (m, 4H), 2.76 (m, 1H), 2.35 (s, broad, 4H), 2.00 (m, 2H), 1.78 (m, 2H), 1.59 (m, 2H); MS (m/z)=428 (M+1).

### Example 35

### 5-(3-Cyclobutyl-2,3,4,5-tetrahydro-1H-benzo[d]azepin-7-yloxy)-2-methyl-2H-pyridazin-3-one

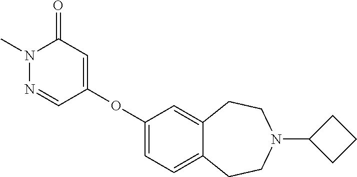

### General Synthesis of Example 35

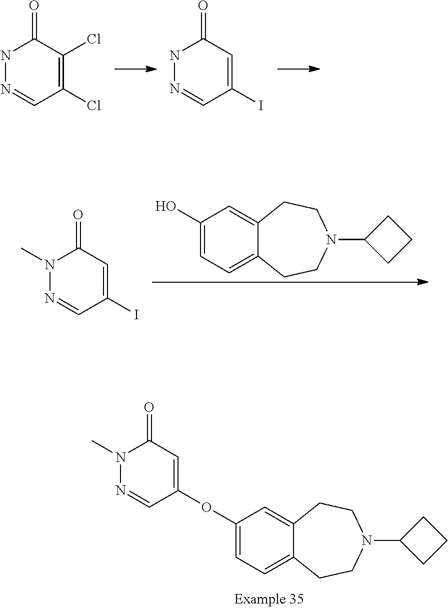

### Step 1: Synthesis of 5-iodo-2H-pyridazin-3-one

A round-bottom flask contained 4,5-dichloro-2H-pyridazin-3-one (10.0 g, 60.6 mmol) and hydriodic acid (57% in H₂O, 80 mL, 606 mmol) was heated at 145° C. for 27 h. After cooled to room temperature, the black precipitation was collected by filtration and was washed with water. The black solid was stirred in water (50 mL) and was added solid sodium thiosulfate (Na₂O₃S₂) in portion until the black color turned grey. The solid material was collected by filtration and was dissolved in 150 mL solvent (MeOH/CH₂Cl₂ 1:1). It was filtered and the filtrate was concentrated and dried to give 6.13 g (46%) of 5-iodo-2H-pyridazin-3one. ¹HNMR (400 MHz, DMSO-d₆) δ 13.25 (s, 1H), 8.08 (s, 1H), 7.54 (s, 1H); MS (m/z)=222 (M+1).

### Step 2: Synthesis of 5-iodo-2-methyl-2H-pyridazin-3-one

To a stirring mixture of 5-iodo-2H-pyridazin-3-one (500 mg, 2.25 mmol) and potassium carbonate (622 mg, 4.50 mmol) in acetonitrile (20 mL) was added iodomethane (150 μL, 2.41 mmol). The reaction was heated at reflux for 1 h and was cooled to room temperature. The reaction was filtered through a pad of silica gel and eluted with methanol. The filtrate was concentrated and the residue was purified by column chromatography (1% MeOH/CH₂Cl₂) to give 355 mg (67%) of 5-iodo-2-methyl-2H-pyridazin-3-one. ¹HNMR (400 MHz, DMSO-d₆) δ 8.12 (s, 1H), 7.57 (s, 1H), 3.58 (s, 3H)

### Step 3. Synthesis of Example 35 Tartaric Acid Salt

To a round-bottom flask was added 5-iodo-2-methyl-2H-pyridazin-3-one (351 mg, 1.49 mmol), 3-cyclobutyl-2,3,4,5-tetrahydro-1H-benzo[d]azepin-7-ol hydrochloride acid (396 mg, 1.56 mmol), potassium carbonate (618 mg, 4.47 mmol) and DMF (10 mL). The reaction mixture was heated 100° C. for 24 h and was cooled to room temperature. The reaction was filtered through a pad of Celite and eluted with solvent (MeOH/CH₂Cl₂ 1:1). The filtrate was concentrated and the residue was purified by column chromatography (2% MeOH/CH₂Cl₂) to give the desired product. This material was converted to its tartaric acid salt as 5-(3-cyclobutyl-2,3,4,5-tetrahydro-1H-benzo[d]azepin-7-yloxy)-2-methyl-2H-pyridazin-3-one tartaric acid. ¹HNMR (400 MHz, DMSO-d₆) δ 7.97 (m, 1H), 7.29 (m, 1H), 7.09 (s, 1H), 7.02 (m, 1H), 5.79 (m, 1H), 4.41 (m, 1H), 4.20 (s, 2H), 3.65 (s, 3H), 3.59 (s, 3H), 3.25 (m, 1H), 2.99 (m, 4H), 2.79 (s, broad, 4H), 2.16-2.00 (m, 4H), 1.70-1.56 (m, 2H); MS (m/z) 326 (M+1).

### Example 36

### 5-(3-Cyclopentyl-2,3,4,5-tetrahydro-1H-benzo[d]azepin-7-yloxy)-3-methyl-2H-pyridazin-3-one

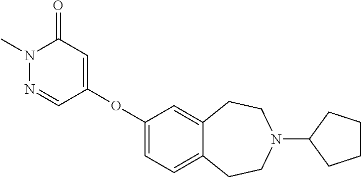

This compound was synthesized using methods for Example 35.

¹HNMR (400 MHz, DMSO-d₆) δ 7.97 (m, 1H), 7.28 (m, 1H), 7.07 (s, 1H), 7.00 (m, 1H), 5.80 (m, 1H), 4.38 (m, 1H), 4.18 (m, 1H), 4.06 (s, 2H), 3.65 (s, 2H), 3.59 (s, 3H), 3.23 (m, 1H), 3.05-2.90 (m, 8H), 1.87 (m, 2H), 1.65-1.47 (m, 6H); MS (m/z)=340 (M+1).

### Example 37

### 5-(3-Cyclobutyl-2,3,4,5-tetrahydro-1H-benzo[d]azepin-7-yloxy)-2-methoxymethyl-2H-pyridazin-3-one

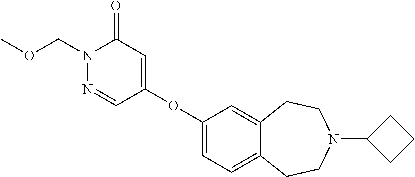

This compound was synthesized using methods for Example 35.

¹HNMR (400 MHz, DMSO-d₆) δ 8.04 (m, 1H), 7.27 (m, 1H), 7.08 (s, 1H), 7.02 (m, 1H), 5.75 (m, 1H), 5.26 (s, 2H), 4.40 (m, 1H), 4.23 (m, 1H), 4.11 (s, 2H), 3.65 (m, 2H), 3.31 (s, 3H), 3.03 (m, 1H), 2.94 (m, 4H), 2.61 (s, broad, 4H), 2.06 (m, 2H), 1.94 (m, 2H), 1.66-1.55 (m, 2H); MS (m/z)=356 (M+1).

### Example 38

### 5-(3-Cyclopentyl-2,3,4,5-tetrahydro-1H-benzo[d]azepin-7-yloxy)-2-methoxymethyl-2H-pyridazin-3-one

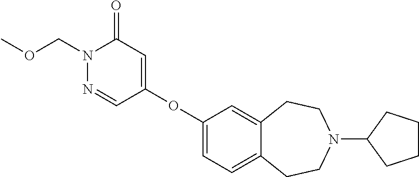

This compound was synthesized using methods for Example 35.

¹HNMR (400 MHz, DMSO-d₆) δ 8.05 (m, 1H), 7.29 (m, 1H), 7.10 (s, 1H), 7.03 (m, 1H), 5.77 (m, 1H), 5.27 (s, 2H), 4.38 (s, 1H), 4.18 (s, 1H), 4.06 (s, 2H), 3.65 (m, 2H), 3.31 (s, 3H), 3.23 (m, 1H), 3.06-2.90 (m, 8H), 1.88 (m, 2H), 1.68-1.45 (m, 6H); MS (m/z)=370 (M+1).

### Example 39

### 5-(2,3,4,5-Tetrahydro-1H-benzo[d]azepin-7-yl)-3,4-diaza-bicyclo[4.1.0]hept-4-en-2-one

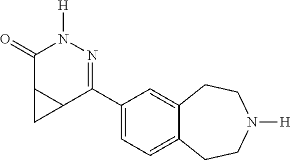

In a 25 mL round bottom flask, 2,2,2-trifluoro-1-(1,2,4,5-tetrahydro-3-benzapin-3-yl)-ethanone (500 mg, 2.1 mmol), and 3-oxabicyclo[3.1.0]hexane-2,4-dione (230 mg, 2.1 mmol), were added to 1,2-dichloroethane (10 mL) at 0° C. Aluminum chloride (822 mg, 6.2 mmol) was added slowly, the ice bath removed and the mixture was heated to 85° C. 14 h. The reaction was cooled and concentrated under vacuum. Water (10 mL) and ammonium hydroxide (5 mL) were added and the slurry stirred vigorously 4 h. The alumina salts were filtered off. The filtrate was transferred to a 50 mL flask, hydrazine monohydrate (0.21 mL, 4.2 mmol), and the reaction heated to 110° C. 14 h. After cooling, the organics were extracted three times with methylene chloride, dried over MgSO₄, and then concentrated under vacuum. HCl salt formation (MeOH/ether) produced 78 mg (13%). mp 148-151° C.: ¹H NMR (400 MHz, CDCl₃) δ 10.10 (bs, 2H), 8.30 (s, 1H), 7.63 (s, 1H), 7.62 (d, J=8.0 Hz, 1H), 7.25 (d, J=8.0 Hz, 1H), 3.34 bs, 8H), 2.54 (m, 1H), 2.23 (m, 1H), 1.89 (m, 1H), 1.21 (dd, J₁=J₂=7.0 Hz, 1H) MS m/z=256 (M+1).

### Example 40

### 5-(3-Cyclobutyl-2,3,4,5-tetrahydro-1H-benzo[d]azepin-7-yl)-3,4-diaza-bicyclo[4.1.0]hept-4-en-2-one

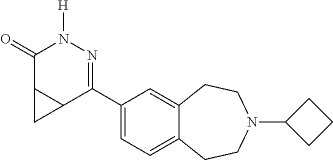

In a 10 mL round bottom flask, 5-(2,3,4,5-tetrahydro-1H-benzo[d]azepin-7-yl)-3,4-diaza-bicyclo[4.1.0]hept-4-en-2-one; hydrochloride (72 mg, 0.25 mmol), cyclobutanone (0.04 mL, 0.49 mmol), and acetic acid (0.1 mL), were added to methanol (5 mL). Sodium cyanoborohydride (78 mg, 1.2 mmol) was added slowly, and the mixture was heated to 60° C. 14 h. The reaction was cooled and concentrated under vacuum. Saturated sodium bicarbonate solution (5 mL) was added, the slurry was extracted three times with methylene chloride and then dried over MgSO₄. Purification with silica gel chromatography eluting with methylene chloride/methanol (95:5), followed by HCl salt formation produced 13 mg (15%). mp 129-132° C.: ¹H NMR (400 MHz, DMSO-d₆) δ 10.83 (s, 1H), 10.32 (bs, 1H), 7.68 (s, 1H), 7.64 (d, J=8.0 Hz, 1H), 7.31 (d, J=8.0 Hz, 1H), 3.63 (m, 1H), 3.51 (m, 1H), 3.07 (m, 2H), 2.78 (m, 2H), 2.20 (m, 4H), 1.73 (m, 8H), 0.75 (m, 1H), MS m/z=310 (M+1).

### Example 41

### 4-(2,3,4,5-Tetrahydro-1H-benzo[d]azepin-7-yl)-2,4-a,5,6,7,7a-hexahydro-cyclopenta[d]pyridazin-1-one

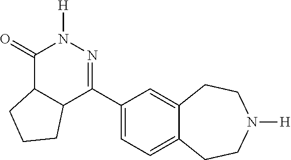

### Step 1

In a 1-neck round-bottom flask under an atmosphere of nitrogen (1S,2R)-cyclopentane-1,2-dicarboxylic acid monomethyl ester (2.00 g, 0.0116 mol;) in methylene chloride (15 mL, 0.23 mol) was added 3-4 drops DMF and oxalyl chloride (1.03 mL, 0.0122 mol) dropwise. The reaction was stirred overnight at rt then was concentrated.

### Step 2

2,2,2-Trifluoro-1-(1,2,4,5-tetrahydro-3-benzazepin-3-yl)-ethanone in 1,2-dichloroethane (50 mL, 0.6 mol) was cooled to at 0° C. and aluminum trichloride (4.65 g, 0.0348 mol) was added. After 0.5 h the acid chloride was added in 10 mL DCE. After stirring at rt 20 h, 2N HCl was added and the product extracted with DCM and washed with water, NaCl solution and dried (MgSO₄). The solvent was concentrated and the oil was used directly in the next step.

### Step 3

2,2,2-Trifluoro-1-(1,2,4,5-tetrahydro-3-benzazepin-3-yl)-ethanone (2.82 g, 0.0116 mol) intermediate in isopropyl alcohol (30 mL, 0.4 mol) was added hydrazine hydrate (2 mL, 0.04 mol). The reaction was heated at 90° C. for 20 h and cooled to rt. The solvent was concentrated and the crude product was chromatographed using 5-10% MeOH/DCM on to give 0.6 g of product. The HCl salt was prepared by adding 2 mL of 2N HCl/ether to an ether solution of the product: mp>135 dec.; MS m/z=284 (M+1).

### Example 42

### 4-(3-Cyclobutyl-2,3,4,5-tetrahydro-1H-benzo[d]azepin-7-yl)-2,4a,5,6,7,7a-hexahydro-cyclopenta[d]pyridazin-1-one

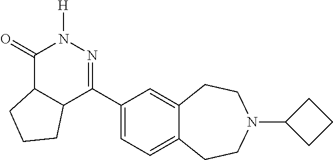

Into a 1-neck round-bottom flask, 4-(2,3,4,5-tetrahydro-1H-3-benzazepin-7-yl)-2,4-a,5,6,7,7a-hexahydro-cyclopenta[d]pyridazin-1-one (0.5 g, 2.0 mmol) in methanol (25 mL) was added cyclobutanone (0.3 mL, 4.0 mmol) and sodium cyanoborohydride (0.9 g, 10 mmol). Acetic acid (1 mL, 20 mmol) was added and the reaction was stirred at 60° C. for 12 h. After cooling to rt, the mixture was poured into 1 M sodium carbonate solution (20 mL) and was extracted with EtOAc (2×50 mL). The solvent was dried over MgSO₄ and concentrated. The product was chromatographed on an ISCO 40 g silica column using 5-10% MeOH/DCM. The fractions containing product were collected and triturated with ether; yield 500 mg; mp 198-201° C., ¹HNMR (CDCl₃): 8.5 (s, 1H), 7.5 (s, 1H), 7.47 (d, 1H, J=7.8 Hz), 7.16 (d, 1H, J=7.9 Hz), 3.32 (m, 1H), 2.86-3.0 (m, 7H), 2.54 (bs, 5H), 2.23 (m, 1H), 1.95-2.15 (m, 7H), 1.6-1.7 (m, 2H); MS m/z=338 (M+1).

### Example 43

### 4-(2,3,4,5-Tetrahydro-1H-benzo[d]azepin-7-yl)-4a,5,6,7,8,8a-hexahydro-2H-phthalazin-1-one

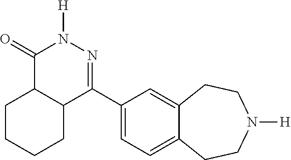

This compound was prepared using the method described for Example 41. Oil at rt. MS m/z=298 (M+1).

### Example 44

### 4-(3-Cyclobutyl-2,3,4,5-tetrahydro-1H-benzo[d]azepin-7-yl)-4-a,5,6,7,8,8a-hexahydro-2H-phthalazin-1-one

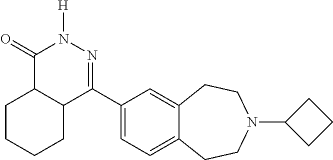

This compound was prepared using the method described for Example 42. mp=223-225° C.; NMR (CDCl₃): 8.49 (s, 1H), 7.53 (s, 1H), 7.47 (d, 1H, J=7.8), 7.15 (d, 1H, J=7.8 Hz); MS m/z=352 (M+1).

### Example 45

### 2-Methyl-6-[4-((S)-2-pyrrolidin-1-ylmethyl-pyrrolidine-1-carbonyl)-phenyl]-2H-pyridazin-3-one

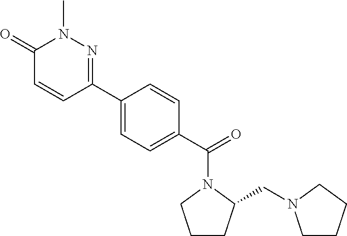

4-(1-Methyl-6-oxo-1,6-dihydropyridazin-3-yl)benzoic acid (0.875 g, 3.8 mmol) was heated with SO₂Cl₂ (5 mL) at 70° C. for 1 h. The volatiles were removed by rotary evaporation followed by high vacuum. The crude acid chloride was dissolved in CH₂Cl₂ (5 mL) and added to an ice cold solution of (S)-2-pyrrolidin-1-ylmethyl-pyrrolidine (0.643 g, 4.17 mmol) in CH₂Cl₂ (10 mL), then stirred at rt for 2 h. The mixture was diluted with CH₂Cl₂, partitioned with saturated NaHCO₃ solution, brine, then dried over Na₂SO₄ and evaporated. The product was purified by ISCO using CH₂Cl₂/MeOH/NH₄OH as eluent to give the compound as a yellowish foam. (1 g, 72%). ¹HNMR (400 MHz, at 90° C. DMSO): δ 7.96 (d, J=9.6 Hz, 1H), 7.89 (d, J=6.6 Hz, 2H), 7.54 (d, J=6.6 Hz, 2H), 7.00 (d, J=9.6 Hz, 1H), 4.20 (brs, 1H), 3.75 (s, 3H), 3.43 (s, 2H), 1.7-2.6 (m, 10H), 1.6 (s, 4H). MS m/z 367 (M+1).

### Example 46

### 6-[4-(4-Cyclopentyl-piperazine-1-carbonyl)-phenyl]-2-methyl-2H-pyridazin-3-one

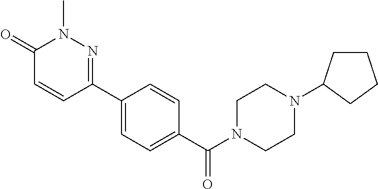

This compound was synthesized using N-cyclopentyl piperazine by the method described for Example 45. mp 177-179° C. ¹HNMR (400 MHz, CDCl₃) δ 7.83 (d, J=7.1 Hz, 2H), 7.7 (d, J=9.6 Hz, 1H), 7.51 (d, J=7.1 Hz, 2H), 7.05 (d, J=9.6 Hz, 1H), 3.9 (s, 3H), 3.8 (brs, 2H), 3.48 (brs, 2H), 2.4-2.7 (m, 5H), 1.35-1.95 (m, 8H); MS m/z=367 (M+1).

### Example 47

### 6-[4-(4-Isopropyl-piperazine-1-carbonyl)-phenyl]-2-methyl-2H-pyridazin-3-one

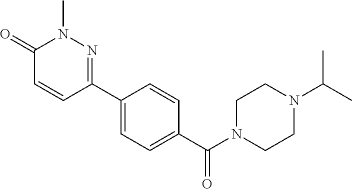

This compound was synthesized using N-isopropyl piperazine by the method described for Example 45. mp 131-132° C. ¹HNMR (400 MHz, CDCl₃): δ 7.85 (d, J=7.0 Hz, 2H), 7.7 (d, J=9.6 Hz, 1H), 7.5 (d, J=7.0 Hz, 2H), 7.05 (d, J=9.6 Hz, 1H), 3.9 (s, 3H), 3.8 (brs, 2H), 3.5 (brs, 2H), 2.7 (m, 1H) & 2.6 (brs, 2H), 2.5 (brs, 2H), 1.07 (d, J=6.3 Hz, 3H); MS m/z=341 (M+1).

### Example 48

### 6-[4-((S)-Hexahydro-pyrrolo[1,2-a]pyrazine-2-carbonyl)-phenyl]-2-methyl-2H-pyridazin-3-one

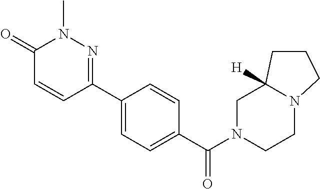

This compound was synthesized using 4-(S)-hexahydro-pyrrolo[1,2-a]pyrazine by the method described for Example 45. ¹HNMR (400 MHz, CDCl₃) δ 7.85 (d, J=8.0 Hz, 2H), 7.7 (d, J=10.0 Hz, 1H), 7.5 (d, J=8.0 Hz, 2H), 7.05 (d, J=10.0 Hz, 1H), 4.8 (m, 1H), 3.9 (s, 3H), 3.8 (m, 1H) and 1.3-3.3 (m, 11H); MS m/z=339 (M+1).

### Example 49

### 2-(4-Fluoro-phenyl)-6-[4-((S)-2-pyrrolidin-1-ylmethyl-pyrrolidine-1-carbonyl)-phenyl]-2H-pyridazin-3-one

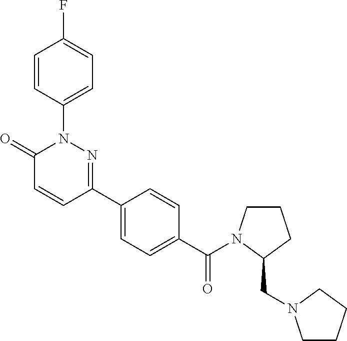

This compound was synthesized using 4-[1-(4-fluorophenyl)-6-oxo-1,6-dihydro-pyridazin-3-yl]-benzoic acid by the method described for Example 45. HCl salt, mp 177° C. ¹HNMR (400 MHz, DMSO): δ 10.0 (brs, 1H), 8.2 (d, J=9.9 Hz, 1H), 8.0 (d, J=8.0 Hz, 2H), 7.7 (m, 4H), 7.4 (m, 2H), 7.2 (d, J=9.9 Hz, 1H), 4.5 (m, 1H), 3.0-3.8 (m, 8H), 1.7-2.2 (m, 8H); MS m/z=447 (M+1).

### Example 50

### 6-[4-((S)-2-Pyrrolidin-1-ylmethyl-pyrrolidine-1-carbonyl)-phenyl]-2H-pyridazin-3-one

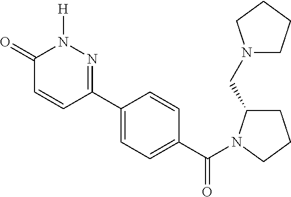

This compound was synthesized using 4-[6-oxo-1,6-dihydro-pyridazin-3-yl]-benzoic acid by the method described for Example 45. mp 62-66° C. ¹HNMR (400 MHz, CDCl₃): δ 7.9 (d, J=8.0 Hz, 2H), 7.8 (d, J=9.9 Hz, 1H), 7.6 (brs, 2H), 7.1 (d, J=9.9 Hz, 1H), 4.5 (brs, 1H), 2.6-3.6 (m, 8H), 2.5-1.5 (m, 8H). MS m/z=353 (M+1).

### Example 51

### 6-{4-[2-((R)-2-Methyl-pyrrolidin-1-yl)-ethyl]-phenyl}-2H-pyridazin-3-one

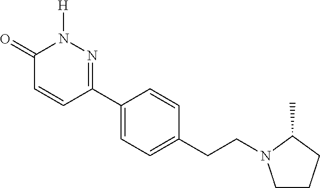

### Step 1: Synthesis of Acetic acid 2-[4-(6-oxo-1,4,5,6-tetrahydro-pyridazin-3-yl)-phenyl]-ethyl ester

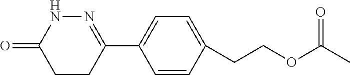

A solution of 4-[4-(2-acetoxy-ethyl)-phenyl]-4-oxo-butyric acid (2.00 g, 7.57 mmol) in ethanol (20 mL) and hydrazine monohydrate (0.42 g, 8.40 mmol) was heated at 100° C. overnight. The reaction mixture was evaporated under vacuum and triturated with a mixture of ether and hexane to produce acetic acid-2-[4-(6-oxo-1,4,5,6-tetrahydro-pyridazin-3-yl)-phenyl]-ethyl ester (0.80 g, 41%) as a white solid, mp 94-96° C. (ether and hexane), MS m/z=261 (M+1).

### Step 2: Synthesis of acetic acid 2-[4-(6-oxo-1,6-dihydro-pyridazin-3-yl)-phenyl]-ethyl ester

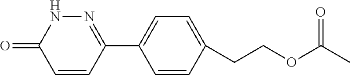

To a solution of acetic acid-2-[4-(6-oxo-1,4,5,6-tetrahydro-pyridazin-3-yl)-phenyl]-ethyl ester (1.28 g, 4.92 mmol) in acetonitrile (15 mL) was added copper(II) chloride (1.30 g, 9.70 mmol). The reaction mixture was heated to reflux for 30 min, concentrated under vacuum and then suspended in water. The reaction mixture was extracted four times with 2% methanol in ethyl acetate. The combined organics was washed with water, brine, dried (Na₂SO₄), filtered and concentrated under vacuum to produce acetic acid-2-[4-(6-oxo-1,6-dihydro-pyridazin-3-yl)-phenyl]-ethyl ester (1.22 g, 96%) as a light yellow solid, mp 186° C. (ethyl acetate and methanol), MS m/z −257 (M−1).

### Step 3: Synthesis of 6-[4-(2-Hydroxy-ethyl)-phenyl]-2H-pyridazin-3-one

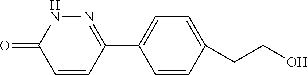

A solution of acetic acid-2-[4-(6-oxo-1,6-dihydro-pyridazin-3-yl)-phenyl]-ethyl ester (1.22 g, 4.72 mmol) in a mixture of methanol (25 mL) and tetrahydrofuran (20 mL) was added potassium carbonate (1.96 g, 14.20 mmol) and stirred at room temperature for overnight. The reaction mixture was concentrated under vacuum and neutralized with 1N HCl to pH 4 and the resulting solid was filtered, washed with water and dried. The dried solid was again treated with 1N HCl and stirred at room temperature then filtered, washed with water and dried to produce 6-[4-(2-hydroxy-ethyl)-phenyl]-2H-pyridazin-3-one (0.65 g, 64%) as a tan solid. The crude product was used for the next reaction without further purification.

### Step 4

A solution of 6-[4-(2-hydroxy-ethyl)-phenyl]-2H-pyridazin-3-one (0.65 g, 3.00 mmol) in methylene chloride (10 ml) was cooled to 0° C. and added triethyl amine (1.82 g, 18.01 mmol) and methanesulfonyl chloride (0.89 g, 7.80 mmol). The reaction mixture was stirred at 0° C. for 1 h and at room temperature for 2 h then concentrated under vacuum to dryness. The residue was partitioned between saturated aqueous sodium bicarbonate solution and ethyl acetate. The aqueous layer was extracted three times with ethyl acetate and the combined organics was washed with brine, dried (Na₂SO₄), filtered and concentrated to produce the disulfonate (1.00 g, 90%), MS m/z=373 (M+1). This crude material was used directly in the next reaction without further purification.

### Step 5: Synthesis of 6-{4-[2-((R)-2-Methyl-pyrrolidin-1-yl)-ethyl]-phenyl}-2H-pyridazin-3-one

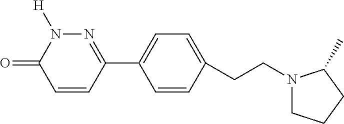

A mixture of the product from step 4 (1.00 g, 2.68 mmol), (R)-2-methyl pyrrolidine.tartarate (3.78 g, 16.08 mmol) and potassium carbonate (2.58 g, 18.69 mmol) in acetonitrile (20 mL) was stirred at 50° C. for 6 days. The reaction mixture was concentrated under vacuum then partitioned between saturated aqueous sodium bicarbonate solution and methylene chloride. The aqueous phase was extracted three times with methylene chloride and the combined organics was washed with brine, dried (Na₂SO₄), filtered and concentrated to afford a crude product. The crude product was purified by Biotage chromatography using 3% to 10% methanol in methylene chloride to 10% methanol containing 2 mL of ammonium hydroxide in methylene chloride to afford a relatively product. The product was crystallized from a mixture of methylene chloride, ethanol, ether and hexane to produce 6-{4-[2-(R)-2-methyl-pyrrolidine-1-yl)-ethyl]-phenyl}-2H-pyridazin-3-one (0.15 g, 20%), mp 159-161° C. (methylene chloride, ethanol, ether, and hexane), MS m/z=284 (M+1), ¹H NMR (400 MHz, CDCl₃, δ): 1.135 (d, J=6.07 Hz, 3H), 1.42-1.55 (m, 1H), 1.69-2.02 (m, 3H), 2.18-2.28 (m, 1H), 2.30-2.42 (m, 2H), 2.83-2.97 (m, 2H), 3.02-3.13 (m, 1H), 3.23-3.33 (m, 1H), 7.06 (d, J=9.85 Hz, 1H), 7.34 (d, J=8.04 Hz, 2H), 7.71 (d, J=8.33 Hz, 2H), 7.745 (d, J=9.85 Hz, 1H), 11.00-11.11 (br S, 1H).

The following Examples were synthesized using the general procedure for Example 51.

| Example | Structure | MS (M + 1) |
|---|---|---|
| 51 | 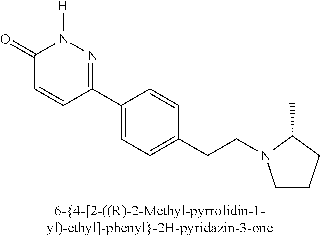 | 284 |
| 52 | 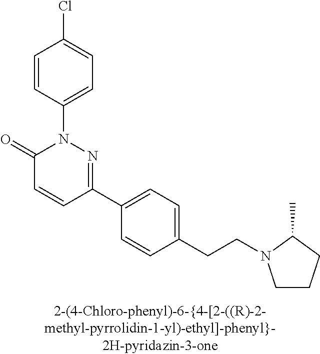 | 394 |
| 53 | 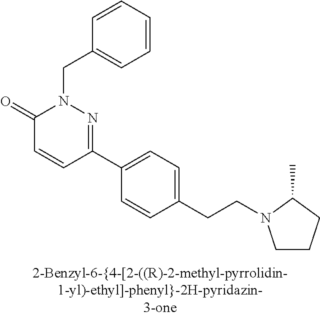 | 374 |
| 54 | 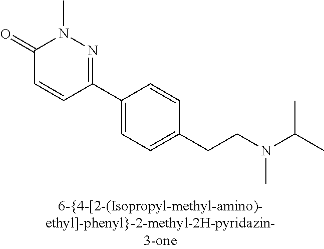 | 286 |
| 55 | 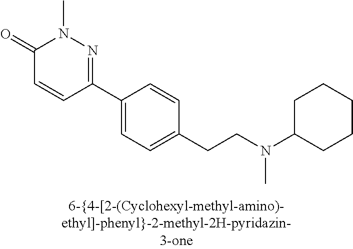 | 326 |
| 56 | 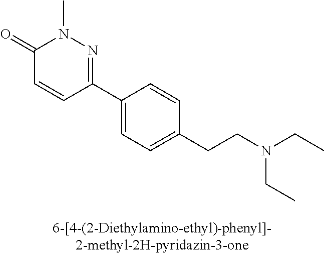 | 286 |
| 57 | 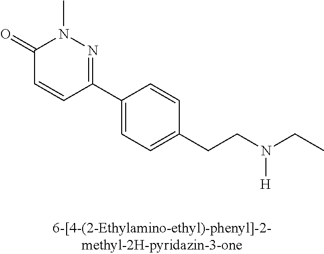 | 258 |
| 58 | 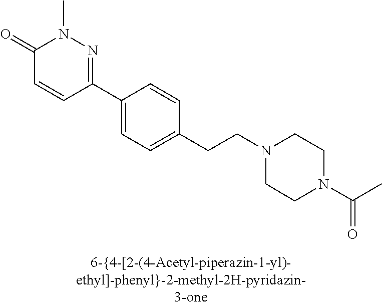 | 341 |
| 59 | 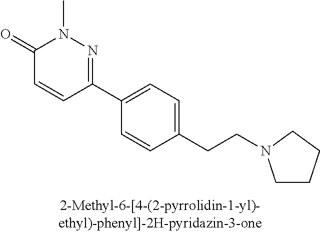 | 384 |
| 60 | 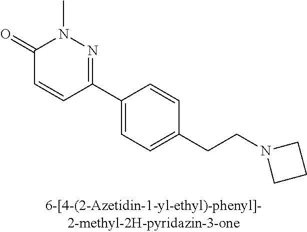 | 270 |
| 61 | 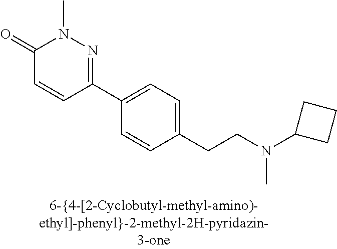 | 298 |
| 62 | 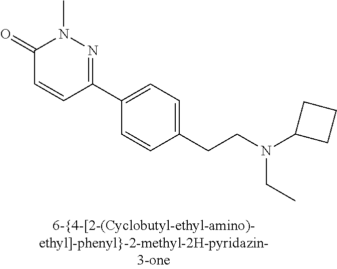 | 312 |
| 63 | 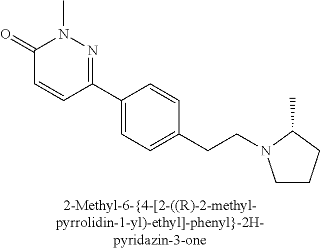 | 298 |
| 64 | 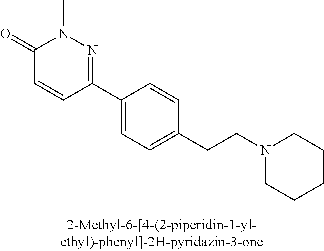 | 298 |
| 65 | 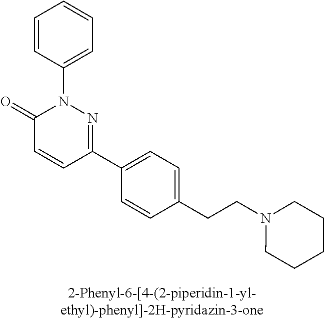 | 360 |
| 66 | 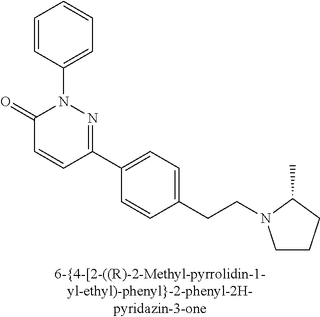 | 360 |
| 67 | 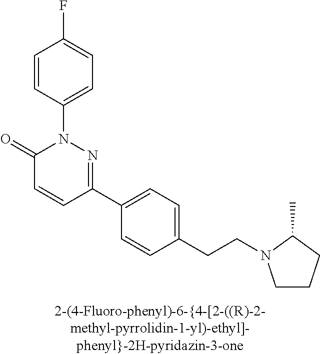 | 378 |
| 68 | 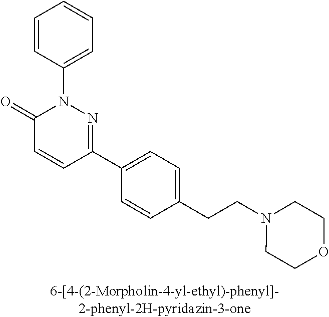 | 362 |
| 69 | 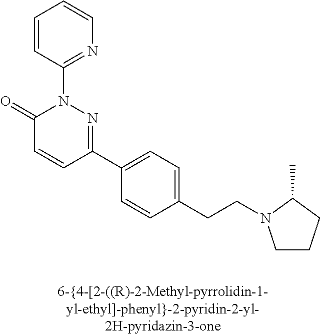 | 361 |

### Example 70

### 6-{6-[2-((R)-2-Methyl-pyrrolidin-1-yl)-ethyl]-naphthalen-2-yl}-2H-pyridazin-3-one

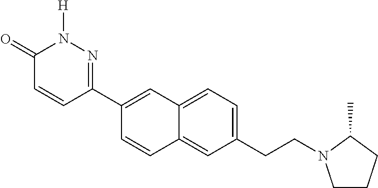

(R)-2-Methyl-1-{2-[6-(4,4,5,5-tetramethyl-[1,3,2]dioxaborolan-2-yl)-naphthalen-2-yl]-ethyl}-pyrrolidine (0.267 g, 0.731 mmol), 3-tert-butoxy-6-chloro-pyridazine (0.18 g, 0.97 mmol), tetrakis(triphenylphosphine)palladium(0) (0.05912 g, 0.051 mmol), potassium carbonate (0.273 g, 1.97 mmol), 1,2-dimethoxyethane (10 mL), and water (4 mL) were combined in a round bottom flask and degassed with argon. The reaction was heated at 85° C. overnight and was monitored by MS and HPLC. After the reaction had completed, the product mixture was cooled to room temperature, filtered through celite, washed with saturated sodium bicarbonate, and extracted with methylene chloride. The combined organics were dried over sodium sulfate, filtered, and concentrated.

The crude material was taken up in 10 mL methylene chloride and trifluoroacetic acid (1 mL) was added. This was stirred at room temperature for 5 h until HPLC indicated that the reaction had reached completion. The crude material was concentrated, cooled to 0° C. with an ice bath, and sodium bicarbonate was added. This was extracted three times with methylene chloride, and then concentrated. The material was purified on ISCO (40 g) 0-15% methanol in methylene chloride. The fractions containing product material were combined, concentrated, and dissolved in methanol. Ethereal HCl was added to form the HCl salt and crystallized using methanol and ether. The product material was then dried in a ChemDry to give 99 mg of purified material. mp 295-8° C. LCMS m/z=334.08 (M+1).

### Utility

The compounds of the present invention are useful, inter alia, as therapeutic agents. Particularly, the compounds are useful for interacting with the H₃ receptor. In one embodiment, the present invention provides a method for treating or preventing diseases and disorders, such as those disclosed herein, which comprises administering to a subject in need of such treatment or prevention a therapeutically effective amount of a compound of the present invention.

In an additional embodiment, the present invention provides a method for inhibiting H₃ activity comprising providing a compound of the present invention in an amount sufficient to result in effective inhibition. Particularly, the compounds of the present invention can be administered to treat such diseases and disorders such as narcolepsy or other sleep/wake disorders, such as obstructive sleep apnea/hypopnea syndrome, and shift work sleep disorder; feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders (such as asthma), inflammation, and myocardial infarction. In certain embodiments, the compounds can be administered to treat narcolepsy or other sleep/wake disorders, such as obstructive sleep apnea/hypopnea syndrome, and shift work sleep disorder; obesity, cognition, attention deficit hyperactivity disorder (ADHD), and dementia. In other embodiments, the compounds can be administered to treat narcolepsy or other sleep/wake disorders, such as obstructive sleep apnea/hypopnea syndrome, and shift work sleep disorder; or they can used to treat obesity, or they can used to treat cognition, or they can used to treat attention deficit hyperactivity disorder (ADHD), or they can used to treat dementia.

Compounds of the invention either have demonstrated or are expected to demonstrate inhibition of H₃ and thereby for utility for treatment of the indications described herein. Such utilities can be determined using, for example, the following assays as set forth below. They are not intended, nor are they to be construed, as limiting the scope of the disclosure.

### Rat H₃ Assays:

Cell line development and membrane preparation. The rat H₃ receptor cDNA was PCR amplified from reverse-transcribed RNA pooled from rat thalamus, hypothalamus, striatum and prefrontal cortex with a sequence corresponding to by #338-1672 of Genbank file #NM_{—}053506, encoding the entire 445-amino-acid rat histamine H₃ receptor. This was engineered into the pIRES-neo3 mammalian expression vector, which was stably transfected into the CHO-A3 cell line (Euroscreen, Belgium), followed by clonal selection by limiting dilution. Cells were harvested and cell pellets were frozen (−80° C.). Cell pellets were resuspended in 5 mM Tris-HCl, pH 7.5 with 5 nM EDTA and a cocktail of protease inhibitors (Complete Protease Inhibitor Tablets, Roche Diagnostics). Cells were disrupted using a polytron cell homogenizer and the suspension was centrifuged at 1000×g for 10 minutes at 4° C. The pellet was discarded and the supernatant centrifuged at 40,000×g for 30 min at 4° C. This membrane pellet was washed in membrane buffer containing 50 mM Tris-HCl, pH 7.5 with 0.6 mM EDTA, 5 mM MgCl₂ and protease inhibitors, recentrifuged as above and the final pellet resuspended in membrane buffer plus 250 mM sucrose and frozen at −80° C.

Radioligand Binding. Membranes were resuspended in 50 mM Tris HCl (pH 7.4), 5 mM MgCl₂, 0.1% BSA. The membrane suspensions (10 μg protein per well) were incubated in a 96 well microtiter plate with [³H]-N-alpha-methylhistamine (approximately 1 nM final concentration), test compounds at various concentrations (0.01 nM-30 μM) and scintillation proximity beads (Perkin Elmer, FlashBlueGPCR Scintillating Beads) in a final volume of 80 μl for 4 hours at room temperature, protected from light. Non-specific binding was determined in the presence of 10 μM clobenpropit. Radioligand bound to receptor, and therefore in proximity to the scintillation beads, was measured using a MicroBeta scintillation counter.

GTPγS Binding. Membranes were resuspended in 20 mM HEPES pH 7.4 containing: 1 mM EDTA, 0.17 mg/ml dithiothreitol, 100 mM NaCl, 30 μg/ml saponin and 5 mM MgCl₂. For measurement of inverse agonist activity, increasing concentrations of test compounds were incubated in a 96 well microtiter plate with 10 μg/well membrane protein, 5 μM GDP, scintillation proximity beads (Perkin Elmer, FlashBlueGPCR Scintillating Beads) and [³⁵S]-GTPγS (0.1 nM final concentration). Following incubation for 45 minutes in the dark at room temperature, the microtiter plate was centrifuged at 1000×g for 5 minutes and radioactivity bound to the membranes was counted using a MicroBeta scintillation counter. Non-specific binding was measured in the presence of 10 μM GTP. A decrease in bound [³⁵S]-GTPγS is indicative of H₃ receptor inverse agonist activity in this assay. Antagonist activity of test compounds was determined in a similar experiment under the following conditions. Membranes were resuspended in 20 mM HEPES pH 7.4 containing: 1 mM EDTA, 0.17 mg/ml dithiothreitol, 200 mM NaCl, 30 μg/ml saponin and 20 mM MgCl₂. The membranes were incubated at 10 μg/well membrane protein in a microtiter plate with increasing concentrations of test compounds, 20 μM GDP, scintillation proximity beads and [³⁵S]-GTPγS (0.1 nM final concentration) plus 30 nM R-alpha-methylhistamine. The microtiter plates were incubated and processed as described above. A decrease in R-alpha-methylhistamine stimulated [³⁵S]-GTPγS binding is indicative of H₃ receptor antagonist activity in this assay.

### Human H₃ Assays:

Methods: CHO cells stably expressing the human H₃ receptor (GenBank: NM_{—}007232) were harvested and cell pellets were frozen (−80° C.). Cell pellets were resuspended in 5 mM Tris-HCl, pH 7.5 with 5 nM EDTA and a cocktail of protease inhibitors (Complete Protease Inhibitor Tablets, Roche Diagnostics). Cells were disrupted using a polytron cell homogenizer and the suspension was centrifuged at 1000×g for 10 minutes at 4° C. The pellet was discarded and the supernatant centrifuged at 40,000×g for 30 min at 4° C. This membrane pellet was washed in membrane buffer containing 50 mM Tris-HCl, pH 7.5 with 0.6 mM EDTA, 5 mM MgCl₂ and protease inhibitors, recentrifuged as above and the final pellet resuspended in membrane buffer plus 250 mM sucrose and frozen at −80° C.

Radioligand Binding. Membranes were resuspended in 50 mM Tris HCl (pH 7.4), 5 mM MgCl₂, 0.1% BSA. The membrane suspensions (10 μg protein per well) were incubated in a 96 well microtiter plate with [³H]-N-alpha-methylhistamine (approximately 1 nM final concentration), test compounds at various concentrations (0.01 nM-30 μM) and scintillation proximity beads (Perkin Elmer, FlashBlueGPCR Scintillating Beads) in a final volume of 80 μl for 4 hours at room temperature, protected from light. Non-specific binding was determined in the presence of 10 μM clobenpropit. Radioligand bound to receptor, and therefore in proximity to the scintillation beads, was measured using a MicroBeta scintillation counter.

GTPγS Binding. Membranes were resuspended in 20 mM HEPES pH 7.4 containing: 1 mM EDTA, 0.17 mg/ml dithiothreitol, 100 mM NaCl, 30 μg/ml saponin and 5 mM MgCl₂. For measurement of inverse agonist activity, increasing concentrations of test compounds were incubated in a 96 well microtiter plate with 10 μg/well membrane protein, 5 μM GDP, scintillation proximity beads (Perkin Elmer, FlashBlueGPCR Scintillating Beads) and [³⁵S]-GTPγS (0.1 nM final concentration). Following incubation for 45 minutes in the dark at room temperature, the microtiter plate was centrifuged at 1000×g for 5 minutes and radioactivity bound to the membranes was counted using a MicroBeta scintillation counter. Non-specific binding was measured in the presence of 10 μM GTP. A decrease in bound [³⁵S]-GTPγS is indicative of H₃ receptor inverse agonist activity in this assay. Antagonist activity of test compounds was determined in a similar experiment under the following conditions. Membranes were resuspended in 20 mM HEPES pH 7.4 containing: 1 mM EDTA, 0.17 mg/ml dithiothreitol, 200 mM NaCl, 30 μg/ml saponin and 20 mM MgCl₂. The membranes were incubated at 10 μg/well membrane protein in a microtiter plate with increasing concentrations of test compounds, 20 μM GDP, scintillation proximity beads and [³⁵S]-GTPγS (0.1 nM final concentration) plus 30 nM R-alpha-methylhistamine. The microtiter plates were incubated and processed as described above. A decrease in R-alpha-methylhistamine stimulated [³⁵S]-GTPγS binding is indicative of H₃ receptor antagonist activity in this assay.

Other assays that may be used in connection with the present invention are set forth below. Examples of the present invention can be tested in the following in vivo models:

### Evaluation of Wake Promoting Activity in Rats

The methodology utilized for evaluating wake promoting activity of test compounds is based on that described by Edgar and Seidel, Journal of Pharmacology and Experimental Therapeutics, 283:757-769, 1997, and incorporated herein in its entirety by reference.

Compounds of the invention either have demonstrated or are expected to demonstrate utility for wake promoting activity.

Dipsogenia Model: Inhibition of histamine agonist-induced water drinking in the rat. Histamine, and the H₃-selective agonist (R)-α-methylhistamine (RAMH) induce water drinking behavior in the rat when administered either peripherally or centrally (Kraly, F. S., June, K. R. 1982 Physiol. Behav. 28: 841.; Leibowitz, S. F. 1973 Brain Res. 63:440; Ligneau X., Lin, J-S., Vanni-Mercier G., Jouvet M., Muir J. L., Ganellin C. R., Stark H., Elz S., Schunack W., Schwartz, J-C. 1998 J Pharmcol. Exp. Ther. 287:658-66.; Clapham, J. and Kilpatrick G. J. 1993 Eur. J. Pharmacol. 232:99-103) an effect which is blocked by H₃ receptor antagonists thioperamide and ciproxifan. Compounds of the invention either have demonstrated or are expected to block RAMH induce water drinking behavior. Novel object discrimination: Novel object discrimination (NOD; also referred to as novel object recognition) is an assay for short-term visual recognition memory that was first described by Ennaceur and Delacour (Ennaceur, A. and Delacour, J. (1988) Behav Brain Res 31: 47-59).

Social recognition: Social recognition (SR) is an assay for short-term social (olfactory) memory that was first described by Thor and Holloway (1982). Thor, D. and Holloway, W. (1982) J Comp Physiolog Psychcol 96: 1000-1006.

Table A lists the Human and Rat H₃ binding data for Examples 1-69 of the present invention. Binding constants (Kᵢ) for Examples 1-69 in the Human H₃ and Rat H₃ methods described herein are expressed by letter descriptor to indicate the following ranges: “+++” is less than 100 nM; “++” is 100-1000 nM; “+” is >1000 nM. The descriptor “nd” in Table 1 means not determined.

TABLE A

| Example No. | hH₃ Kᵢ | rH₃ Kᵢ |
|---|---|---|
| 1 | | nd |
| 2 | +++ | +++ |
| 3 | ++ | ++ |
| 4 | +++ | ++ |
| 5 | ++ | ++ |
| 6 | ++ | ++ |
| 7 | +++ | ++ |
| 8 | ++ | ++ |
| 9 | +++ | ++ |
| 10 | +++ | +++ |
| 11 | +++ | +++ |
| 12 | +++ | +++ |
| 13 | +++ | +++ |
| 14 | +++ | +++ |
| 15 | +++ | ++ |
| 16 | +++ | ++ |
| 17 | +++ | +++ |
| 18 | +++ | +++ |
| 19 | +++ | ++ |
| 20 | + | + |
| 21 | +++ | +++ |
| 22 | ++ | ++ |
| 23 | +++ | ++ |
| 24 | + | nd |
| 25 | +++ | ++ |
| 26 | +++ | +++ |
| 27 | +++ | +++ |
| 28 | +++ | +++ |
| 29 | +++ | +++ |
| 30 | +++ | +++ |
| 31 | +++ | +++ |
| 32 | +++ | +++ |
| 33 | +++ | +++ |
| 34 | +++ | ++ |
| 35 | +++ | ++ |
| 36 | +++ | ++ |
| 37 | +++ | ++ |
| 38 | +++ | ++ |
| 39 | ++ | ++ |
| 40 | +++ | ++ |
| 41 | ++ | ++ |
| 42 | +++ | +++ |
| 43 | nd | nd |
| 44 | +++ | +++ |
| 45 | +++ | +++ |
| 46 | ++ | ++ |
| 47 | ++ | ++ |
| 48 | ++ | ++ |
| 49 | +++ | ++ |
| 50 | +++ | +++ |
| 51 | +++ | +++ |
| 52 | +++ | +++ |
| 53 | +++ | +++ |
| 54 | ++ | ++ |
| 55 | ++ | ++ |
| 56 | ++ | ++ |
| 57 | ++ | ++ |
| 58 | ++ | ++ |
| 59 | +++ | ++ |
| 60 | ++ | ++ |
| 61 | +++ | ++ |
| 62 | ++ | ++ |
| 63 | +++ | +++ |
| 64 | ++ | ++ |
| 65 | ++ | ++ |
| 66 | +++ | +++ |
| 67 | +++ | +++ |
| 68 | + | + |
| 69 | +++ | ++ |

### Dosage and Formulation

For therapeutic purposes, the compounds of the present invention can be administered by any means that results in the contact of the active agent with the agent's site of action in the body of the subject. The compounds may be administered by any conventional means available for use in conjunction with pharmaceuticals, either as individual therapeutic agents or in combination with other therapeutic agents, such as, for example, analgesics. The compounds of the present invention are preferably administered in therapeutically effective amounts for the treatment of the diseases and disorders described herein to a subject in need thereof.

A therapeutically effective amount can be readily determined by the attending diagnostician, as one skilled in the art, by the use of conventional techniques. The effective dose will vary depending upon a number of factors, including the type and extent of progression of the disease or disorder, the overall health status of the particular patient, the relative biological efficacy of the compound selected, the formulation of the active agent with appropriate excipients, and the route of administration. Typically, the compounds are administered at lower dosage levels, with a gradual increase until the desired effect is achieved.

Typical dose ranges are from about 0.01 mg/kg to about 100 mg/kg of body weight per day, with a preferred dose from about 0.01 mg/kg to 10 mg/kg of body weight per day. A preferred daily dose for adult humans includes about 25, 50, 100 and 200 mg, and an equivalent dose in a human child. The compounds may be administered in one or more unit dose forms. The unit dose ranges from about 1 to about 500 mg administered one to four times a day, preferably from about 10 mg to about 300 mg, two times a day. In an alternate method of describing an effective dose, an oral unit dose is one that is necessary to achieve a blood serum level of about 0.05 to 20 μg/ml in a subject, and preferably about 1 to 20 μg/ml.

The compounds of the present invention may be formulated into pharmaceutical compositions by admixture with one or more pharmaceutically acceptable excipients. The excipients are selected on the basis of the chosen route of administration and standard pharmaceutical practice, as described, for example, in Remington: The Science and Practice of Pharmacy, 20^{th} ed.; Gennaro, A. R., Ed.; Lippincott Williams & Wilkins: Philadelphia, Pa., 2000. The compositions may be formulated to control and/or delay the release of the active agent(s), as in fast-dissolve, modified-release, or sustained-release formulations. Such controlled-release, or extended-release compositions may utilize, for example biocompatible, biodegradable lactide polymers, lactide/glycolide copolymers, polyoxyethylene-polyoxypropylene copolymers, or other solid or semisolid polymeric matrices known in the art.

The compositions can be prepared for administration by oral means; parenteral means, including intravenous, intramuscular, and subcutaneous routes; topical or transdermal means; transmucosal means, including rectal, vaginal, sublingual and buccal routes; ophthalmic means; or inhalation means. Preferably the compositions are prepared for oral administration, particularly in the form of tablets, capsules or syrups; for parenteral administration, particularly in the form of liquid solutions, suspensions or emulsions; for intranasal administration, particularly in the form of powders, nasal drops, or aerosols; or for topical administration, such as creams, ointments, solutions, suspensions aerosols, powders and the like.

For oral administration, the tablets, pills, powders, capsules, troches and the like can contain one or more of the following: diluents or fillers such as starch, or cellulose; binders such as microcrystalline cellulose, gelatins, or polyvinylpyrrolidones; disintegrants such as starch or cellulose derivatives; lubricants such as talc or magnesium stearate; glidants such as colloidal silicon dioxide; sweetening agents such as sucrose or saccharin; or flavoring agents such as peppermint or cherry flavoring. Capsules may contain any of the afore listed excipients, and may additionally contain a semi-solid or liquid carrier, such as a polyethylene glycol. The solid oral dosage forms may have coatings of sugar, shellac, or enteric agents. Liquid preparations may be in the form of aqueous or oily suspensions, solutions, emulsions, syrups, elixirs, etc., or may be presented as a dry product for reconstitution with water or other suitable vehicle before use. Such liquid preparations may contain conventional additives such as surfactants, suspending agents, emulsifying agents, diluents, sweetening and flavoring agents, dyes and preservatives.

The compositions may also be administered parenterally. The pharmaceutical forms acceptable for injectable use include, for example, sterile aqueous solutions, or suspensions. Aqueous carriers include mixtures of alcohols and water, buffered media, and the like. Nonaqueous solvents include alcohols and glycols, such as ethanol, and polyethylene glycols; oils, such as vegetable oils; fatty acids and fatty acid esters, and the like. Other components can be added including surfactants; such as hydroxypropylcellulose; isotonic agents, such as sodium chloride; fluid and nutrient replenishers; electrolyte replenishers; agents which control the release of the active compounds, such as aluminum monostearate, and various co-polymers; antibacterial agents, such as chlorobutanol, or phenol; buffers, and the like. The parenteral preparations can be enclosed in ampules, disposable syringes or multiple dose vials. Other potentially useful parenteral delivery systems for the active compounds include ethylene-vinyl acetate copolymer particles, osmotic pumps, implantable infusion systems, and liposomes.

Other possible modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for topical use are in the form of an ointment, cream, or gel. Typically these forms include a carrier, such as petrolatum, lanolin, stearyl alcohol, polyethylene glycols, or their combinations, and either an emulsifying agent, such as sodium lauryl sulfate, or a gelling agent, such as tragacanth. Formulations suitable for transdermal administration can be presented as discrete patches, as in a reservoir or microreservoir system, adhesive diffusion-controlled system or a matrix dispersion-type system. Formulations for buccal administration include, for example lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate.

As those skilled in the art will appreciate, numerous modifications and variations of the present invention are possible in light of the above teachings. It is therefore understood that within the scope of the appended claims, the invention may be practiced otherwise than as specifically described herein, and the scope of the invention is intended to encompass all such variations.

### REFERENCES

Alguacil L. F.; Perez-Garcia C. Histamine H₃ Receptor: A potential drug target for the treatment of central nervous systems disorders. Current Drug Targets—CNS & Neurological Disorders 2003, 2, 303-131.
Arrang, J. M.; Garbarg, M.; Schwartz, J. C., Auto-inhibition of brain histamine release mediated by a novel class (H₃) of histamine receptor. Nature 1983, 302, (5911), 832-7.
Celanire, S.; Wijtmans, M.; Talaga, P.; Leurs, R.; de Esch, I. J., Keynote review: histamine H₃ receptor antagonists reach out for the clinic. Drug Discov Today 2005, 10, (23-24), 1613-27.
Chazot P. L.; Hann V. H₃ histamine receptor isoforms: New therapeutic targets in the CNS? Current Opinions in Investigational Drugs 2001, 2, 1428-1431.
Chen Z. Effect of histamine H₃-receptor antagonist clobenprobit on spatial memory of radial maze performance in rats. Acta Pharmacol Sin 2000, 21, 905-910.
Esbenshade, T. A.; ox, G. B.; Cowart, M. D. Histamine H₃ receptor antagonists: Preclinical promise for treating obesity and cognitive disorders. Molecular interventions 2006, 6, 77-88.
Fox G. B.; Pan J. B.; Esbenshade T. A.; Bennani Y. L.; Black L. A.; Faghih R.; Hancock A. A.; Decker M. W. Effects of histamine H₃ receptor ligands GT-2331 and ciproxifan in a repeated acquisition response in the spontaneously hypertensive rat pup. Behav. Brain Res. 2002, 131, 151-161.
Fox G. B.; Pan J. B.; Radek R. J.; Lewis A. M.; Bitner R. S.; Esbenshade T. A.; Faghih R.; Bennani Y. L.; Williams W.; Yao B. B. Decker M. W.; Hancock A. A. Two novel and selective nonimidazole H₃ receptor Antagonists A-304121 and A-317920: II. In vivo behavioral and neurophysiological characterization. J. Pharmacol. Exper. Ther. 2003, 305, 897-908.
Hancock, A. A.; Esbenshade, T. A.; Krueger, K. M.; Yao, B. B., Genetic and pharmacological aspects of histamine H₃ receptor heterogeneity. Life Sci 2003, 73, (24), 3043-72.
Hancock, A. A.; Fox, G. B. Perspectives on cognitive domains, H₃ receptor ligands and neurological disease. Expert Opin. Investig. Drugs, 2004, 13, 1237-1248.
Komater V. A.; Browman K. E.; Curzon P.; Hancock A. A., Decker M. W.; Fox B. H₃ receptor blockade by thioperamide enhances cognition in rats without inducing locomotor sensitization. Psychopharmacology 2003, 167, 363-372.
Leurs R.; Blandina P.; Tedford C.; Timmerman H. Therapeutic potential of histamine H₃ receptor agonists and antagonists. Trends in Pharmacology 1998, 19, 177-183.
Leurs, R.; Bakker, R. A.; Timmerman, H.; de Esch, I. J., The histamine H₃ receptor: from gene cloning to H₃ receptor drugs. Nat Rev Drug Discov 2005, 4, (2), 107-20.
Lin, J. S.; Sakai, K.; Vanni-Mercier, G.; Arrang, J. M.; Garbarg, M.; Schwartz, J. C.; Jouvet, M., Involvement of histaminergic neurons in arousal mechanisms demonstrated with H₃-receptor ligands in the cat. Brain Res 1990, 523, (2), 325-30.
Lloyd G. K.; Williams M. Neuronal nicotinic acetylcholine receptors as novel drug targets. J Pharmacol Exp Ther. 2000, 292, 461-467.
Monti, J. M.; Jantos, H.; Ponzoni, A.; Monti, D., Sleep and waking during acute histamine H₃ agonist BP 2.94 or H₃ antagonist carboperamide (MR 16155) administration in rats. Neuropsychopharmacology 1996, 15, 31-5.
Orsetti M.; Ferretti C.; Gamalero S. R.; Ghi P. Histamine H₃-receptor blockade in the rat nucleus basalis magnocellularis improves place recognition memory. Psychopharmacology 2002, 159, 133-137.
Parmentier R.; Ohtsu H.; Djebbara-Hannas Z.; Valatx J-L.; Watanabe T.; Lin J-S. Anatomical, physiological, and pharmacological characteristics of histidine decarboxylase knock-out mice: evidence for the role of brain histamine in behavioral and sleep-wake control. J. Neurosci. 2002, 22, 7695-7711.
Passani, M. B.; Lin, J. S.; Hancock, A.; Crochet, S.; Blandina, P., The histamine H₃ receptor as a novel therapeutic target for cognitive and sleep disorders. Trends Pharmacol Sci 2004, 25, 618-25.
Repka-Ramirez M. S, New concepts of histamine receptors and actions. Current Allergy and Asthma Reports 2003, 3, 227-231.
Ritz A.; Curley J.; Robertson J.; Raber J. Anxiety and cognition in histamine H₃ receptor −/− mice. Eur J Neurosci 2004, 19, 1992-1996.
Rouleau, A.; Heron, A.; Cochois, V.; Pillot, C.; Schwartz, J. C.; Arrang, J. M., Cloning and expression of the mouse histamine H₃ receptor: evidence for multiple isoforms. J Neurochem 2004, 90, 1331-8.
Vanni-Merci G.; Gigout S.; Debilly G.; Lin J. S. Waking selective neurons in the posterior hypothalamus and their response to histamine H₃-receptor ligands: an electrophysiological study in freely moving cats. Behav Brain Res 2003, 144, 227-241.
Witkin, J. M.; Nelson, D. L., Selective histamine H₃ receptor antagonists for treatment of cognitive deficiencies and other disorders of the central nervous system. Pharmacol Ther 2004, 103, 1-20.
Yao, B. B.; Sharma, R.; Cassar, S.; Esbenshade, T. A.; Hancock, A. A., Cloning and pharmacological characterization of the monkey histamine H₃ receptor. Eur J Pharmacol 2003, 482, (1-3), 49-60.

What is claimed is:

1. A compound or a stereoisomer or a pharmaceutically acceptable salt thereof according to Formula I, II, III, IV, V, VI, VII or VIII: 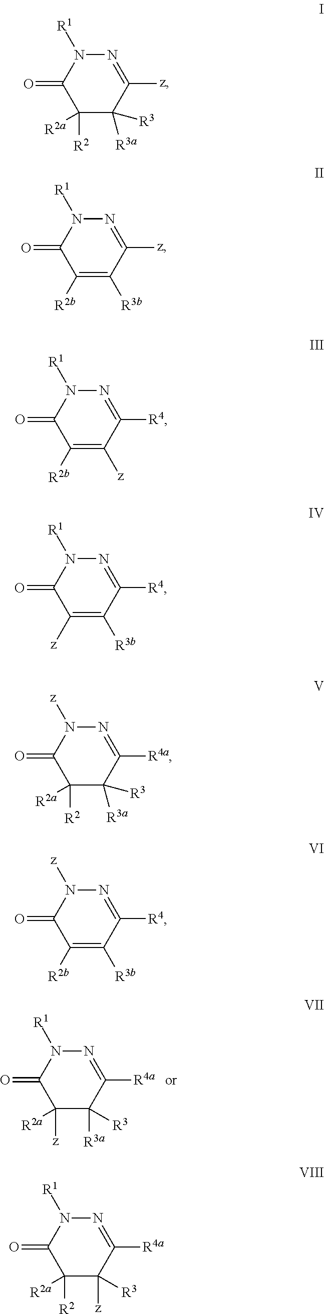
wherein
R¹ is selected from the group consisting of H, C₁₋₆ alkyl, C₂₋₆ alkenyl, C₂₋₆ alkynyl, C₃₋₁₀ cycloalkyl, C₆₋₁₀ aryl, C₇₋₁₈ arylalkyl, 5-10 membered heteroaryl, 3-10 membered heterocycloalkyl, —C(O)R²⁷ and —CO₂R²⁷, wherein the alkyl, cycloalkyl, aryl, arylalkyl, heteroaryl or heterocycloalkyl group is optionally substituted with 1 to 3 R²⁰ groups;
R², R³, R⁴, R^{2a}, R^{2b}, R^{3a}, R^{3b} and R^{4a} are independently selected from the group consisting of H, halo, C₁₋₆ alkyl, C₆₋₁₀ aryl, C₇₋₁₈ arylalkyl, C₁₋₆ alkoxy, —S(O)_{y}—C₁₋₆ alkyl, OR¹¹, C(O)R¹¹, CO₂R¹¹, C(O)NR¹²R¹³ and NR¹²R¹³, C₃₋₁₀ cycloalkyl, 3-10 membered heterocycloalkyl and 5-10 membered heteroaryl, alternatively R^{2b} and R^{3b} or R^{3b} and R⁴ may be taken together to form a C₃₋₁₀ cycloalkyl, 3-10 membered heterocycloalkyl, C₆₋₁₀ aryl or a 5-10 membered heteroaryl; or R² and R³ or R^{2a} and R^{3a} or R^{2a} and R³ or R² and R^{3a} or R^{3a} and R^{4a} or R² and R^{2a} or R³ and R^{3a} or R³ and R^{4a} may be taken together to form a C₃₋₁₀ cycloalkyl or 3-10 membered heterocycloalkyl; provided that no more than one pair of R² and R³, R³ and R^{4a}, R^{2a} and R^{3a}, R^{2a} and R³, R² and R^{3a}, R^{3a} and R^{4a}, R² and R^{2a}, R³ and R^{3a}, R^{2b} and R^{3b} or R^{3b} and R⁴ are taken together with the carbon atoms through which they are connected or to which they are attached to form a ring; and wherein the fused cycloalkyl, heterocycloalkyl, aryl or heteroaryl ring is optionally substituted with 1 to 3 R²⁰ groups;
Z is selected from the group consisting of 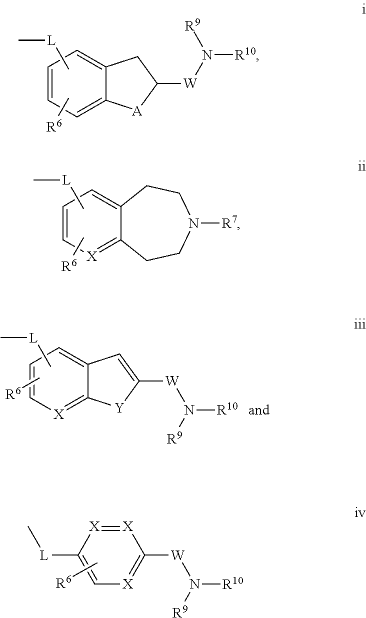
A is selected from the group consisting of C₁₋₃ alkylene, —CH₂—O— and —O—CH₂—;
L is selected from the group consisting of a direct bond, —R²⁵—O— and —O—R²⁵—;
W is selected from C₆₋₁₀ arylene and —C(O)—;
X is independently selected from the group consisting of —C(H)— and —N—;
Y is selected from the group consisting of —N(R³¹)—, —S—, —O—, —C(H)═C(H)— and —C(H)—N(R³¹)—, provided that Y is not —S—, —O— or —C(H)═C(H)— for compounds according to Formulas V and VI;
R⁶ is selected from the group consisting of H, halo, CN, NO₂, C₁₋₆ alkyl, C₂₋₆ alkenyl, C₂₋₆ alkynyl, C₃₋₆ cycloalkyl, 3-10 membered heterocycloalkyl, C₆₋₁₀ aryl, 5-10 membered heteroaryl, C₁₋₆ alkoxy, C₁₋₆ haloalkyl, OR¹¹, C(O)R¹¹, CO₂R¹¹, C(O)NR¹²R¹³ and NR¹²R¹³;
R⁷ is selected from the group consisting of H, C₁₋₆ alkyl, C₂₋₆ alkenyl, C₂₋₆ alkynyl and C₃₋₁₀ cycloalkyl;
R⁹ and R¹⁰ are each independently selected from the group consisting of H, C₁₋₆ alkyl and C₃₋₆ cycloalkyl, wherein said alkyl and cycloalkyl groups may be optionally substituted with 1 to 3 R¹⁴ groups, alternatively R⁹ and R¹⁰ may together with the nitrogen to which they are attached form a 3-10 membered mono- or bicyclo-heterocycloalkyl ring, said heterocycloalkyl ring may be optionally substituted with 1 to 3 R¹⁴ groups;
R¹¹ is selected from the group consisting of H, C₁₋₆ haloalkyl, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said haloalkyl, alkyl, alkenyl, alkynyl, cycloalkyl, aryl, heteroaryl and heterocycloalkyl groups may be optionally substituted with 1 to 3 R²¹ groups;
R¹² and R¹³ are each independently selected from the group consisting of H, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said alkyl, alkenyl, alkynyl, cycloalkyl, aryl, heteroaryl and heterocycloalkyl groups may be optionally substituted with 1 to 3 R²¹ groups, or R¹² and R¹³, together with the nitrogen atom to which they are attached, form a 3-10 membered heterocycloalkyl ring optionally substituted with 1 to 3 R²¹ groups;
R¹⁴ at each occurrence is independently selected from the group consisting of halo, NO₂, CN, —(═O), —C(O)R³⁰, —C(O)OR³⁰, —OC(O)R³⁰, —OC(O)NR²⁸R²⁹, —C(O)NR²⁹R²⁹, —SR³⁰, —S(O)R³⁰, —S(O)₂R³⁰, —S(O)₂NR²⁸R²⁹, NR²⁸R²⁹, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl wherein said alkyl, alkenyl, alkynyl, cycloalkyl, aryl, heteroaryl and heterocycloalkyl groups may be optionally substituted with 1 to 3 R²¹ groups;
R²⁰ at each occurrence is independently selected from the group consisting of F, Cl, Br, I, OR²¹, OR²², NR²³R²⁴, NHOH, NO₂, CN, CF₃, C₁₋₆ alkyl optionally substituted with OR²⁶, C₂₋₆ alkenyl, C₂₋₆ alkynyl, C₃₋₁₀ cycloalkyl, 3-10 membered heterocycloalkyl, C₄₋₁₈ cycloalkylalkyl, 4-18 membered heterocycloalkylalkyl, phenyl, 6-18 membered heteroarylalkyl, C₇₋₁₈ arylalkyl, (═O), C(═O)R²¹, CO₂R²¹, OC(═O)R²¹, C(═O)NR²³R²⁴, NR²⁷C(═O)R²¹, NR²⁷C(═O)OR²¹, OC(═O)NR²³R²⁴, NR²⁷C(═S)R²¹ and S(O)_{q}R²¹;
R²¹ at each occurrence is independently selected from the group consisting of H, C₁₋₆ alkyl, C₆₋₁₀ aryl, 3-10 membered heterocycloalkyl and C₇₋₁₈ arylalkyl;
R²² is independently the residue of an amino acid after the hydroxyl group of the carboxyl group is removed;
R²³ and R²⁴ are independently selected from the group consisting of H, C₁₋₆ alkyl and C₆₋₁₀ aryl, or R²³ and R²⁴, together with the nitrogen atom to which they are attached, form a 3-10 membered heterocycloalkyl ring optionally substituted with ═O;
R²⁵ is selected from the group consisting of a direct bond, C₁₋₆ alkylene, C₆₋₁₀ arylene and 5-10 membered heteroarylene;
R²⁶ is selected from the group consisting of H, C₁-C₆ alkyl, C₆₋₁₀ aryl and C₇₋₁₈ arylalkyl;
R²⁷ is selected from the group consisting of H and C₁-C₆ alkyl;
R²⁸ and R²⁹ are each independently selected from the group consisting of H, C₁₋₆ alkyl and C₃₋₆ cycloalkyl or R²⁸ and R²⁹ may together with the nitrogen to which they are attached form a 3-10 membered mono- or bicyclo-heterocycloalkyl ring;
R³⁰ is selected from the group consisting of H, C₁₋₆ haloalkyl, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl;
R³¹ is selected from the group consisting of H, C₁₋₆ alkyl, C₂₋₆ alkenyl, C₂₋₆ alkynyl, C₃₋₁₀ cycloalkyl, C₆₋₁₀ aryl, 5-10 membered heteroaryl and 3-10 membered heterocycloalkyl, wherein said alkyl, alkenyl, alkynyl, cycloalkyl, aryl, heteroaryl and heterocycloalkyl groups may be optionally substituted with 1 to 3 R²¹ groups,
q is 0, 1 or 2; and
y is 0, 1 or 2.

2. The compound of claim 1, according to Formulas I, II, III, IV, V, VI, VII or VIII, wherein
R^{2b}, R^{3b} and R⁴ are independently selected from the group consisting of H, halo, C₁₋₆ alkyl, C₆₋₁₀ aryl, C₇₋₁₈ arylalkyl, C₁₋₆ alkoxy, —S(O)_{y}—C₁₋₆ alkyl, OR¹¹, C(O)R¹¹, CO₂R¹¹, C(O)NR¹²R¹³ and NR¹²R¹³, C₃₋₁₀ cycloalkyl, 3-10 membered heterocycloalkyl and 5-10 membered heteroaryl; and
R², R^{2a}, R³, R^{3a} and R^{4a} are independently selected from the group consisting of H and C₁₋₆ alkyl, alternatively R^{2b} and R^{3b} or R^{3b} and R⁴ may be taken together to form a C₃₋₁₀ cycloalkyl, 3-10 membered heterocycloalkyl, C₆₋₁₀ aryl or a 5-10 membered heteroaryl; or R^{2a} and R^{3a} or R^{2a} and R³ or R² and R^{3a} or R^{3a} and R^{4a} or R² and R^{2a} or R³ and R^{3a} or R² and R³ or R³ and R^{4a} may be taken together to form a C₃₋₁₀ cycloalkyl or 3-10 membered heterocycloalkyl; provided that no more than one pair of R^{2b} and R^{3b}, R^{3b} and R⁴, R^{2a} and R^{3a}, R^{2a} and R³, R² and R^{3a}, R^{3a} and R^{4a}, R² and R^{2a} or R³ and R^{3a} or R² and R³ or R³ and R^{4a}, are taken together with the carbon atoms through which they are connected or to which they are attached to form a ring; and wherein the fused cycloalkyl, heterocycloalkyl, aryl or heteroaryl ring is optionally substituted with 1 to 3 R²⁰ groups.

3. The compound according to claim 1 of Formulas I, II, III or IV, wherein
R¹ is selected from the group consisting of H, C₁₋₆ alkyl, C₃₋₁₀ cycloalkyl, C₆₋₁₀ aryl and 5-10 membered heteroaryl, wherein the alkyl, cycloalkyl, aryl, heteroaryl group is optionally substituted with 1 to 3 R²⁰ groups;
R^{2b}, R^{3b} and R⁴ are independently selected from the group consisting of H, halo, C₁₋₆ alkyl, C₆₋₁₀ aryl, 5-10 membered heteroaryl and C₃₋₁₀ cycloalkyl;
R², R^{2a}, R³ and R^{3a} are independently selected from the group consisting of H and C₁₋₆ alkyl, or R² and R³ or R² and R^{2a} or R^{2b} and R^{3b} or R^{3b} and R⁴ may be taken together to form a C₃₋₁₀ cycloalkyl; provided that no more than one pair of R² and R³ or R² and R^{2a} or R^{2b} and R^{3b} or R^{3b} and R⁴ are taken together with the carbon atoms through which they are connected or to which they are attached to form a ring; and
R⁶ is selected from the group consisting of H, halo, CN, NO₂, C₁₋₆ alkyl, 5-10 membered heteroaryl, C₁₋₆ alkoxy, C₁₋₆ haloalkyl, OR¹¹, C(O)R¹¹, CO₂R¹¹, C(O)NR¹²R¹³ and NR¹²R¹³.

4. The compound according to claim 3 of Formulas I, II and III.

5. The compound according to claim 4, wherein
Y is selected from the group consisting of —N(R³¹)—, —S— and —C(H)═C(H)—.

6. The compound according to claim 5, wherein
W is —C(O)—.

7. The compound according to claim 6, wherein
R¹ is selected from the group consisting of H and C₁₋₆ alkyl.

8. The compound according to claim 7, wherein
R^{2b}, R^{3b} and R⁴ are independently selected from the group consisting of H, C₁₋₆ alkyl, C₆₋₁₀ aryl, 5-10 membered heteroaryl and C₃₋₁₀ cycloalkyl; and
R², R^{2a}, R³ and R^{3a} are independently selected from the group consisting of H and C₁₋₆ alkyl, or R^{2b} and R^{3b} or R^{3b} and R⁴ may be taken together to form a C₃₋₁₀ cycloalkyl; provided that no more than one pair of R^{2b} and R^{3b} or R^{3b} and R⁴ are taken together with the carbon atoms through which they are connected or to which they are attached to form a ring.

9. The compound according to claim 8, wherein
R⁹ and R¹⁰ are taken together with the nitrogen to which they are attached to form a pyrrolidinyl ring or piperidinyl ring, wherein said ring may be optionally substituted with 1 to 3 R¹⁴ groups

10. The compound according to claim 9, wherein the compound is selected from the group consisting of:
6-(2,3,4,5-Tetrahydro-1H-benzo[d]azepin-7-yl)-2H-pyridazin-3-one;
6-(3-Cyclobutyl-2,3,4,5-tetrahydro-1H-benzo[d]azepin-7-yl)-2H-pyridazin-3-one;
2-Isopropyl-6-(2,3,4,5-tetrahydro-1H-benzo[d]azepin-7-yl)-4,5-dihydro-2H-pyridazin-3-one;
6-(3-Cyclobutyl-2,3,4,5-tetrahydro-1H-benzo[d]azepin-7-yl)-2-isopropyl-4,5-dihydro-2H-pyridazin-3-one;
2-Isopropyl-6-(2,3,4,5-tetrahydro-1H-benzo[d]azepin-7-yl)-2H-pyridazin-3-one;
2-Isopropyl-6-(3-isopropyl-2,3,4,5-tetrahydro-1H-benzo[d]azepin-7-yl)-4,5-dihydro-2H-pyridazin-3-one;
6-(3-Cyclobutyl-2,3,4,5-tetrahydro-1H-benzo[d]azepin-7-yl)-2-isopropyl-2H-pyridazin-3-one;
2-Isopropyl-6-(3-isopropyl-2,3,4,5-tetrahydro-1H-benzo[d]azepin-7-yl)-2H-pyridazin-3-one;
6-(3-Cyclobutyl-2,3,4,5-tetrahydro-1H-benzo[d]azepin-7-yl)-4,5-dihydro-2H-pyridazin-3-one;
6-(3-Cyclobutyl-2,3,4,5-tetrahydro-1H-benzo[d]azepin-7-yl)-2-(2,2,2-trifluoro-ethyl)-4,5-dihydro-2H-pyridazin-3-one;
6-(3-Cyclobutyl-2,3,4,5-tetrahydro-1H-benzo[d]azepin-7-yl)-2-(2,2,2-trifluoro-ethyl)-2H-pyridazin-3-one;
6-(3-Cyclobutyl-2,3,4,5-tetrahydro-1H-benzo[d]azepin-7-yl)-2-ethyl-4,5-dihydro-2H-pyridazin-3-one;
2-Butyl-6-(3-cyclobutyl-2,3,4,5-tetrahydro-1H-benzo[d]azepin-7-yl)-4,5-dihydro-2H-pyridazin-3-one;
2-Butyl-6-(3-cyclobutyl-2,3,4,5-tetrahydro-1H-benzo[d]azepin-7-yl)-2H-pyridazin-3-one;
6-(3-Cyclobutyl-2,3,4,5-tetrahydro-1H-benzo[d]azepin-7-yl)-2-isobutyl-4,5-dihydro-2H-pyridazin-3-one;
6-(3-Cyclobutyl-2,3,4,5-tetrahydro-1H-benzo[d]azepin-7-yl)-2-isobutyl-2H-pyridazin-3-one;
6-(3-Cyclobutyl-2,3,4,5-tetrahydro-1H-benzo[d]azepin-7-yl)-2-(2-hydroxy-ethyl)-4,5-dihydro-2H-pyridazin-3-one;
6-(3-Cyclobutyl-2,3,4,5-tetrahydro-1H-benzo[d]azepin-7-yl)-2-(2-hydroxy-ethyl)-2H-pyridazin-3-one;
2-Cyclobutyl-6-(3-cyclobutyl-2,3,4,5-tetrahydro-1H-benzo[d]azepin-7-yl)-4,5-dihydro-2H-pyridazin-3-one;
2-Methyl-6-(2,3,4,5-tetrahydro-1H-benzo[d]azepin-7-yl)-2H-pyridazin-3-one;
6-(3-Cyclobutyl-2,3,4,5-tetrahydro-1H-benzo[d]azepin-7-yl)-2-methyl-2H-pyridazin-3-one;
6-(3-Isopropyl-2,3,4,5-tetrahydro-1H-benzo[d]azepin-7-yl)-2-methyl-2H-pyridazin-3-one. HCl;
6-(3-Cyclopentyl-2,3,4,5-tetrahydro-1H-benzo[d]azepin-7-yl)-2-methyl-2H-pyridazin-3-one. HCl;
2-(4-Methanesulfonyl-phenyl)-6-(2,3,4,5-tetrahydro-1H-benzo[d]azepin-7-yl)-2H-pyridazin-3-one;
6-(3-Cyclobutyl-2,3,4,5-tetrahydro-1H-benzo[d]azepin-7-yl)-2-(4-methanesulfonyl-phenyl)-2H-pyridazin-3-one;
6-[6-(3-Cyclobutyl-2,3,4,5-tetrahydro-1H-benzo[d]azepin-7-yloxy)-pyridin-3-yl]-2-methyl-2H-pyridazin-3-one;
6-[6-(3-Cyclobutyl-2,3,4,5-tetrahydro-1H-benzo[d]azepin-7-yloxy)-pyridin-3-yl]-2H-pyridazin-3-one;
6-[6-(3-Cyclobutyl-2,3,4,5-tetrahydro-1H-benzo[d]azepin-7-yloxy)-pyridin-3-yl]-2-pyridin-2-yl-2H-pyridazin-3-one;
6-[6-(3-Cyclobutyl-2,3,4,5-tetrahydro-1H-benzo[d]azepin-7-yloxy)-pyridin-3-yl]-2-isopropyl-2H-pyridazin-3-one;
4-Chloro-5-(3-cyclobutyl-2,3,4,5-tetrahydro-1H-benzo[d]azepin-7-yloxy)-2-methyl-2H-pyridazin-3-one;
4-Bromo-5-(3-cyclobutyl-2,3,4,5-tetrahydro-1H-benzo[d]azepin-7-yloxy)-2-methyl-2H-pyridazin-3-one;
4-Bromo-5-(3-cyclopentyl-2,3,4,5-tetrahydro-1H-benzo[d]azepin-7-yloxy)-2-methyl-2H-pyridazin-3-one;
4-Chloro-5-(3-cyclobutyl-2,3,4,5-tetrahydro-1H-benzo[d]azepin-7-yloxy)-2-pyridin-2-yl-2H-pyridazin-3-one;
4-Chloro-5-(3-cyclobutyl-2,3,4,5-tetrahydro-1H-benzo[d]azepin-7-yloxy)-2-(2,2,2-trifluoro-ethyl)-2H-pyridazin-3-one;
5-(3-Cyclobutyl-2,3,4,5-tetrahydro-1H-benzo[d]azepin-7-yloxy)-2-methyl-2H-pyridazin-3-one;
5-(3-Cyclopentyl-2,3,4,5-tetrahydro-1H-benzo[d]azepin-7-yloxy)-2-methyl-2H-pyridazin-3-one;
5-(3-Cyclobutyl-2,3,4,5-tetrahydro-1H-benzo[d]azepin-7-yloxy)-2-methoxymethyl-2H-pyridazin-3-one;
5-(3-Cyclopentyl-2,3,4,5-tetrahydro-1H-benzo[d]azepin-7-yloxy)-2-methoxymethyl-2H-pyridazin-3-one;
5-(2,3,4,5-Tetrahydro-1H-benzo[d]azepin-7-yl)-3,4-diaza-bicyclo[4.1.0]hept-4-en-2-one;
5-(3-Cyclobutyl-2,3,4,5-tetrahydro-1H-benzo[d]azepin-7-yl)-3,4-diaza-bicyclo[4.1.0]hept-4-en-2-one;
4-(2,3,4,5-Tetrahydro-1H-benzo[d]azepin-7-yl)-2,4-a,5,6,7,7a-hexahydro-cyclopenta[d]pyridazin-1-one;
4-(3-Cyclobutyl-2,3,4,5-tetrahydro-1H-benzo[d]azepin-7-yl)-2,4-a,5,6,7,7a-hexahydro-cyclopenta[d]pyridazin-1-one;
4-(2,3,4,5-Tetrahydro-1H-benzo[d]azepin-7-yl)-4-a,5,6,7,8,8a-hexahydro-2H-phthalazin-1-one;
4-(3-Cyclobutyl-2,3,4,5-tetrahydro-1H-benzo[d]azepin-7-yl)-4-a,5,6,7,8,8a-hexahydro-2H-phthalazin-1-one;
2-Methyl-6-[4-((S)-2-pyrrolidin-1-ylmethyl-pyrrolidine-1-carbonyl)-phenyl]-2H-pyridazin-3-one;
6-[4-(4-Cyclopentyl-piperazine-1-carbonyl)-phenyl]-2-methyl-2H-pyridazin-3-one;
6-[4-(4-Isopropyl-piperazine-1-carbonyl)-phenyl]-2-methyl-2H-pyridazin-3-one;
6-[4-((S)-Hexahydro-pyrrolo[1,2-a]pyrazine-2-carbonyl)-phenyl]-2-methyl-2H-pyridazin-3-one;
2-(4-Fluoro-phenyl)-6-[4-((S)-2-pyrrolidin-1-ylmethyl-pyrrolidine-1-carbonyl)-phenyl]-2H-pyridazin-3-one; and
6-[4-((S)-2-Pyrrolidin-1-ylmethyl-pyrrolidine-1-carbonyl)-phenyl]-2H-pyridazin-3-one;
or a salt thereof.

11. A pharmaceutical composition, comprising:
at least one compound according to claim 1 or a salt thereof; and
at least one pharmaceutically acceptable carrier or excipient.

12. The pharmaceutical composition according to claim 11, further comprising:
at least one additional therapeutic agent.

13. A method for treating a disorder selected from the group consisting of narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders, inflammation, and myocardial infarction comprising administering to a subject in need of such treatment a therapeutically effective amount of at least one compound of claim 1 or a salt thereof.

14. The method according to claim 13 wherein the disorder is narcolepsy or sleep/wake disorders.

15. The method according to claim 13 wherein the disorder is attention deficit hyperactivity disorder.

16. The method according to claim 13 wherein the disorder is a cognitive disorder.

17. The method according to claim 13 wherein the disorder is Alzheimer's disease/dementia.

18. The method according to claim 13, further comprising:
administering to a subject in need of such treatment a therapeutically effective amount of at least one additional therapeutic agent.

19. The method according to claim 18, wherein the at least one additional therapeutic agent is administered before, after or concurrently with the at least one compound of claim 1 or salt thereof.
